(19) United States
(12) Patent Application Publication
(10) Pub. No.: US 2022/0081409 A1
(43) Pub. Date: Mar. 17, 2022

(54) PDE9 INHIBITOR AND USE THEREOF

(71) Applicant: NANJING TRANSTHERA BIOSCIENCES CO., LTD., NANJING (CN)

(72) Inventors: Zhonghui Wan, NANJING (CN); Di Wu, NANJING (CN)

(21) Appl. No.: 17/420,997

(22) PCT Filed: Jan. 7, 2020
(86) PCT No.: PCT/CN2020/070707

§ 371 Date: Jul. 6, 2021

(30) Foreign Application Priority Data
Jan. 8, 2019 (CN) 201910016146.8

(51) Int. Cl. C07D 401/04; C07D 401/14; C07D 417/14; C07D 405/14; C07D 493/10; A61K 45/06

(52) U.S. Cl. CPC C07D 401/04; C07D 401/14; A61K 45/06; C07D 405/14; C07D 493/10; C07D 417/14

(57) ABSTRACT

The present invention relates to the technical field of pharmaceuticals, and particularly to a PDE9 inhibitor compound of formula (I) or a pharmaceutically acceptable salt or an isomer thereof. The present invention also relates to pharmaceutical formulations, pharmaceutical compositions and use thereof. R₁, R₂, ring A, L, m and n are defined as in the specification. The compound of the present invention can be used in the manufacture of a medicament for treating or preventing the PDE9-mediated related disease.

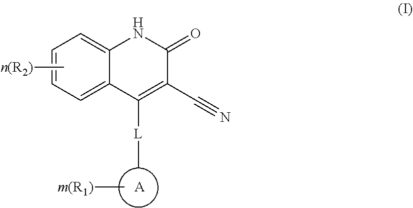

### TECHNICAL FIELD

The present invention relates to the technical field of pharmaceuticals, and particularly to a phosphodiesterase 9 inhibitor compound of formula (I) or a pharmaceutically acceptable salt or an isomer thereof, and use thereof.

### BACKGROUND

Phosphodiesterases (PDEs) are a class of proteases that selectively degrade important second messengers cGMP (cyclic guanosine monophosphate) and cAMP (cyclic adenosine monophosphate) in the body and thus participate in important physiological processes in the body. PDEs can be divided into 11 members (PDE1-PDE11) based on their sequence homology of genes and selectivity for cGMP or cAMP. Among them, PDE9A is an important member of the PDE family, and it is widely expressed in testis, brain, small intestine, skeletal muscle, heart, lung, thymus and pancreas. With the deepening research in recent years, it has been reported in several articles and proved by clinical data that PDE9A inhibitors are useful in the treatment of diseases associated with cognitive impairment caused by central nervous system disorders, such as senile dementia, schizophrenia, and neurodegenerative diseases of the brain.

The two nucleotides, cAMP and cGMP, are important second messengers and play a central role in cell signaling. They mainly activate protein kinases, where the one activated by cAMP is called protein kinase A (PKA) and the one activated by cGMP is called protein kinase G (PKG). Activated PKA and PKG can phosphorylate many cellular effector proteins, such as ion channels, G-protein coupled receptors, structural proteins and transduction factors. Thus, cAMP and cGMP may control most physiological processes in many organs in this way. Meanwhile, cAMP and cGMP can also directly act on effector proteins, thereby playing the same role as described above. It is well known that cGMP can act directly on ion receptors, thereby affecting the ion concentration in cells. PDEs hydrolyze cyclic monophosphates cAMP and cGMP and thus convert them to inactive monophosphates AMP and GMP.

Human PDE9 was first cloned and sequenced in 1998 and is the PDE having the highest selectivity for cGMP reported to date. PDE9 has a binding constant (Km) of 170 nM for cGMP, while it has a binding constant of up to 230,000 nM for cAMP with a selectivity over 1000 times. Compared with PDE2A and PDE5A, PDE9 inhibitors may increase baseline cGMP concentration because PDE9 has no cGMP binding region and thus the catalytic activity of PDE9 is not enhanced by cGMP.

Conventional PDE inhibitors cannot inhibit human PDE9, and therefore the medicaments IBMX, dipyridamole, SKF94120, rolipram and vinpocetine have no inhibitory activity or low inhibitory activity against PDE9.

Currently, no PDE9 inhibitor medicament is available on the market, and only some PDE9 inhibitors are in clinical development phase, such as PF-04447943 by Pfizer (WO2008139293A1, Example 111) and BI-409306 by BI (WO2009121919 A1, Example 51), currently in phase I and phase II clinical stages.

In addition, Merck also reports compounds having PDE9-inhibiting activity in patents WO2017019723A1, WO2017019726A1 and WO2017019724A1. Compound 1-8, as described in WO2017019723A1, has the following structure:

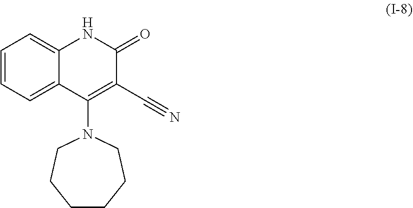 [SMILES: N#Cc1c(N2CCCCCC2)c2ccccc2[nH]c1=O]

### SUMMARY

One purpose of the present invention is to provide a class of compounds or pharmaceutically acceptable salts or isomers thereof used as PDE9 protease inhibitors. The compounds disclosed herein have good inhibitory activity against PDE9 protease, selectivity and druggability (e.g., higher stability in liver microsomes), can treat or prevent PDE9-mediated related diseases, and can play an important role in treating diseases associated with cognitive impairment caused by central nervous system disorders.

The technical solution of the present invention is as follows:

A compound of general formula (I) or a pharmaceutically acceptable salt or an isomer thereof:

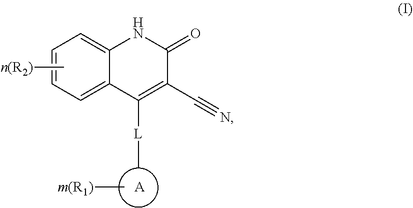

Wherein

each R₂ is independently selected from hydrogen, hydroxy, amino, carboxyl, cyano, nitro, halogen, C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂aminocarbonyl, aryl, 5-6 membered heteroaryl, 4-6 membered heterocyclylcarbonyl and 5-6 membered heteroaryl-oxy, wherein the C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂aminocarbonyl, aryl, 5-6 membered heteroaryl, 4-6 membered heterocyclylcarbonyl and 5-6 membered heteroaryl-oxy are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂amino, C₁₋₆ alkylcarbonylamino, C₁₋₆ alkylsulfonylamino, C₁₋₆ alkylcarbonyloxy, C₃₋₆ cycloalkyl, C₂₋₈ alkynyl, halogenated C₁₋₆ alkyl, C₂₋₈ alkenyl, halogenated C₁₋₆ alkoxy, 4-6 membered heterocyclyl unsubstituted or optionally substituted with a substituent, and heteroaryl unsubstituted or optionally substituted with a substituent;

the substituent in the above 4-6 membered heterocyclyl optionally substituted with a substituent and heteroaryl optionally substituted with a substituent is selected from hydroxy, amino, carboxyl, cyano, nitro, halogen, C₁₋₆ alkyl and C₁₋₆ alkoxy;

L is a bond or —NH—(CH₂)t-, wherein t is 0, 1, 2 or 3;

ring A is 3-8 membered monocyclic heterocyclyl, 6-12 membered bridged heterocyclyl, 6-12 membered spiro-heterocyclyl, 6-12 membered ortho-fused heterocyclyl, aryl, 5-10 membered heteroaryl, 3-12 membered cycloalkyl or 3-12 membered cycloalkenyl, wherein the heterocyclyl has heteroatoms selected from one of or any combinations of O, S and N, the S atom may be optionally oxidized to S(O) or S(O)₂, the C atom may be optionally oxidized to C(O), and the 5-10 membered heteroaryl has heteroatoms selected from one of or any combinations of O, S and N;

each R₁ is independently selected from hydrogen, hydroxy, amino, carboxyl, cyano, nitro, halogen, C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein the C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are unsubstituted or optionally substituted with a group selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino and C₁₋₆ alkylsulfonylamino;

m and n are each independently 0, 1, 2 or 3;

when ring A is 3-8 membered monocyclic heterocyclyl, R₂ is not hydrogen;

when ring A is phenyl, L is not a bond; and

when ring A is

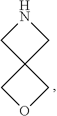

R₂ is not hydrogen.

Some embodiments of the present invention relate to a compound of formula (I) or a pharmaceutically acceptable salt or an isomer thereof, wherein

each R₂ is independently selected from hydrogen, amino, carboxyl, cyano, halogen, C₁₋₆ alkyl, C₁₋₆ alkoxy, C₁₋₆ alkylamino, (C₁₋₆ alkyl)₂amino, C₂₋₈ alkenyl, C₂₋₈ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl, wherein the C₁₋₆ alkyl, C₁₋₆ alkoxy, C₁₋₆ alkylamino, (C₁₋₆ alkyl)₂amino, C₂-8 alkenyl, C₂₋₈ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl are unsubstituted or optionally substituted with one or more groups independently selected from hydroxy, amino, cyano, halogen, C₁₋₆ alkyl, C₁₋₆ alkoxy, C₁₋₆ alkylamino, (C₁₋₆ alkyl)₂amino, C₁₋₆ alkylcarbonyloxy, C₃₋₆ cycloalkyl, and 4-6 membered heterocyclyl unsubstituted or optionally substituted with C₁₋₆ alkyl;

L is a bond;

ring A is 3-8 membered monocyclic heterocyclyl, 6-12 membered bridged heterocyclyl, 6-12 membered spiro-heterocyclyl, 6-12 membered ortho-fused heterocyclyl, phenyl or 5-10 membered heteroaryl, wherein the heterocyclyl has heteroatoms selected from one of or any combinations of O, S and N, the S atom may be optionally oxidized to S(O) or S(O)₂, and the C atom may be optionally oxidized to C(O);

each R₁ is independently selected from hydrogen, hydroxy, cyano, halogen, C₁₋₆ alkyl, C₁₋₆ alkoxy and 5-6 membered heteroaryl, wherein the C₁₋₆ alkyl, C₁₋₆ alkoxy and 5-6 membered heteroaryl are unsubstituted or substituted with hydroxy;

m and n are each independently 0, 1 or 2;

when ring A is 3-8 membered monocyclic heterocyclyl, R₂ is not hydrogen;

when ring A is phenyl, L is not a bond; and

when ring A is

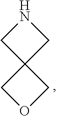

R₂ is not hydrogen.

Some embodiments of the present invention relate to a compound of formula (I) or a pharmaceutically acceptable salt or an isomer thereof, wherein

each R₂ is independently selected from hydrogen, halogen, C₁₋₆ alkyl, C₁₋₆ alkoxy and C₁₋₆ alkylaminocarbonyl, wherein the C₁₋₆ alkyl, C₁₋₆ alkoxy and C₁₋₆ alkylaminocarbonyl are unsubstituted or optionally substituted with one or more groups independently selected from hydroxy, C₁₋₆ alkoxy and C₃₋₆ cycloalkyl;

L is a bond;

ring A is 3-8 membered monocyclic heterocyclyl or 6-12 membered spiro-heterocyclyl, wherein the heterocyclyl has heteroatoms selected from one of or any combinations of O, S and N, the S atom may be optionally oxidized to S(O) or S(O)₂, and the C atom may be optionally oxidized to C(O);

each R₁ is independently selected from hydrogen, C₁₋₆ alkyl and C₁₋₆ alkoxy;

m and n are each independently 0, 1, 2 or 3;

when ring A is 3-8 membered monocyclic heterocyclyl, R₂ is not hydrogen; and

when ring A is

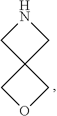

R₂ is not hydrogen.

Some embodiments of the present invention relate to a compound of formula (I) or a pharmaceutically acceptable salt or an isomer thereof, wherein

L is a bond;

ring A is 4-7 membered monocyclic heterocyclyl, wherein the 4-7 membered monocyclic heterocyclyl has heteroatoms selected from one of or combinations of two of O, S and N, and contains at least one N, ring A is connected to L via the N atom, the S atom may be optionally oxidized to S(O) or S(O)₂, and the C atom may be optionally oxidized to C(O);

preferably, ring A is 4-7-membered saturated nitrogen-containing monocyclic heterocyclyl, more preferably

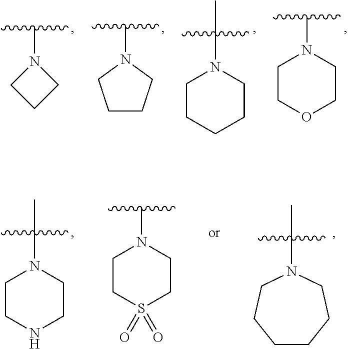

and further more preferably

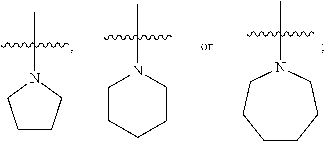 [SMILES: *N1CCCC1 ; *N1CCCCC1 ; *N1CCCCCC1]

each R₂ is independently selected from halogen, C₁₋₄ alkyl, C₁₋₄ alkoxy, morpholinyl, C₂₋₆ alkenyl, C₁₋₄ alkylcarbonyl, C₁₋₄ alkylaminocarbonyl, (C₁₋₄ alkyl)₂aminocarbonyl and aminocarbonyl, wherein the C₁₋₄ alkyl, C₁₋₄ alkoxy, morpholinyl, C₂₋₆ alkenyl, C₁₋₄ alkylcarbonyl, C₁₋₄ alkylaminocarbonyl, (C₁₋₄ alkyl)₂aminocarbonyl and aminocarbonyl are unsubstituted or optionally substituted with one or more groups independently selected from hydroxy, C₁₋₄ alkyl, C₁₋₄ alkoxy, C₃₋₆ cycloalkyl, amino, C₁₋₄ alkylamino, (C₁₋₄ alkyl)₂amino, and 4-6 membered heterocyclyl unsubstituted or optionally substituted with C₁₋₄ alkyl;

each R₁ is independently selected from hydrogen, halogen, C₁₋₄ alkyl, C₁₋₄ alkoxy, pyrazolyl, thiazolyl and triazolyl, wherein the C₁₋₄ alkyl, C₁₋₄ alkoxy, pyrazolyl, thiazolyl and triazolyl are unsubstituted or substituted with hydroxy; and

m and n are each independently 0, 1 or 2.

Some embodiments of the present invention relate to a compound of formula (I) or a pharmaceutically acceptable salt or an isomer thereof, wherein

each R₂ is independently selected from halogen, C₁₋₄ alkyl, C₁₋₄ alkoxy, C₂₋₆ alkenyl, C₁₋₄ alkylcarbonyl, C₁₋₄ alkylaminocarbonyl and aminocarbonyl, wherein the C₁₋₄ alkyl, C₁₋₄ alkoxy, C₂₋₆ alkenyl, C₁₋₄ alkylcarbonyl, C₁₋₄ alkylaminocarbonyl and aminocarbonyl are unsubstituted or optionally substituted with one or more groups independently selected from hydroxy, C₁₋₄ alkyl, C₁₋₄ alkoxy, cyclopropyl, and 4-6 membered heterocyclyl unsubstituted or optionally substituted with C₁₋₄ alkyl;

L is a bond;

ring A is

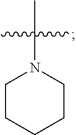 [SMILES: *N1CCCCC1]

each R₁ is independently selected from hydrogen, C₁₋₄ alkyl and C₁₋₄ alkoxy; and

m and n are each independently 0, 1 or 2.

Some embodiments of the present invention relate to a compound of formula (I) or a pharmaceutically acceptable salt or an isomer thereof, wherein

each R₂ is independently selected from halogen, C₁₋₄ alkyl, C₁₋₄ alkoxy and C₁₋₄ alkylaminocarbonyl, wherein the C₁₋₄ alkyl, C₁₋₄ alkoxy and C₁₋₄ alkylaminocarbonyl are unsubstituted or optionally substituted with one or more groups independently selected from hydroxy, C₁₋₄ alkoxy and cyclopropyl;

L is a bond;

ring A is

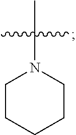 [SMILES: *N1CCCCC1]

each R₁ is independently selected from hydrogen, C₁₋₄ alkyl and C₁₋₄ alkoxy; and

m and n are each independently 0, 1 or 2.

Some embodiments of the present invention relate to a compound of formula (I) or a pharmaceutically acceptable salt or an isomer thereof, wherein

each R₂ is independently selected from amino, carboxyl, cyano, halogen, C₁₋₆ alkyl, C₁₋₆ alkoxy, C₁₋₆ alkylamino, (C₁₋₆ alkyl)₂amino, C₂₋₈ alkenyl, C₂₋₈ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl, wherein the C₁₋₆ alkyl, C₁₋₆ alkoxy, C₁₋₆ alkylamino, (C₁₋₆ alkyl)₂amino, C₂₋₈ alkenyl, C₂₋₈ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl are unsubstituted or optionally substituted with one or more groups independently selected from hydroxy, amino, cyano, halogen, C₁₋₆ alkyl, C₁₋₆ alkoxy, C₁₋₆ alkylamino, (C₁₋₆ alkyl)₂amino, C₁₋₆ alkylcarbonyloxy, C₃₋₆ cycloalkyl, and 4-6 membered heterocyclyl unsubstituted or substituted with C₁₋₆ alkyl;

L is a bond;

ring A is

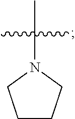 [SMILES: *N1CCCC1]

and

each R₁ is independently selected from pyrazolyl, thiazolyl and triazolyl.

Some embodiments of the present invention relate to a compound of formula (I) or a pharmaceutically acceptable salt or an isomer thereof, wherein

L is a bond;

each R₂ is independently selected from hydrogen, amino, cyano, halogen, carboxyl, C₁₋₄ alkyl, C₁₋₄ alkoxy, C₁₋₄ alkylcarbonyl, C₂₋₆ alkynyl, C₁₋₄ alkylaminocarbonyl, (C₁₋₄ alkyl)₂aminocarbonyl, C₁₋₄ alkylthio, C₁₋₄ alkylsulfonyl, C₁₋₄ alkylamino, (C₁₋₄ alkyl)₂amino, azetidinyl, morpholinyl, piperazinyl, C₂₋₆ alkenyl and cyclopropyl, wherein the C₁₋₄ alkyl, C₁₋₄ alkoxy, C₁₋₄ alkylcarbonyl, C₂₋₆ alkynyl, C₁₋₄ alkylaminocarbonyl, (C₁₋₆ alkyl)₂aminocarbonyl, C₁₋₄ alkylthio, C₁₋₄ alkylsulfonyl, C₁₋₄ alkylamino, (C₁₋₄ alkyl)₂amino, azetidinyl, morpholinyl, piperazinyl, C₂₋₆ alkenyl and cyclopropyl are unsubstituted or optionally substituted with one or more groups independently selected from hydroxy, amino, halogen, C₁₋₄ alkyl, C₁₋₄ alkoxy, C₁₋₄ alkylamino, (C₁₋₄ alkyl)₂amino, cyclopropyl and C₁₋₄ alkylcarbonyloxy;

ring A is 7-12 membered spiro-heterocyclyl, wherein the spiro-heterocyclyl has heteroatoms selected from one of or combinations of two of O, S and N, and contains at least one N, ring A is connected to L via the N atom, the S atom may be optionally oxidized to S(O) or S(O)₂, and the C atom may be optionally oxidized to C(O); preferably, the 7-12 membered spiro-heterocyclyl is a 7-12 membered saturated nitrogen-containing spiro-heterocyclyl; and more preferably, the 7-12 membered saturated nitrogen-containing spiro-heterocyclyl is selected from the following groups:

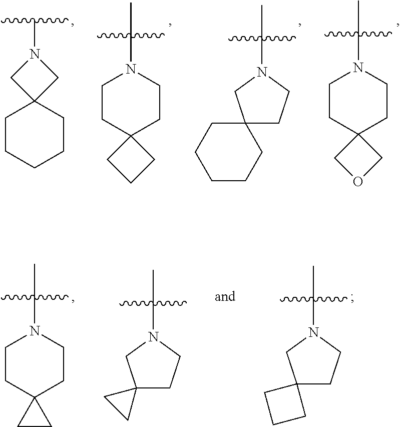

and

when ring A is

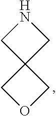

R₂ is not hydrogen.

In some embodiments, ring A is selected from

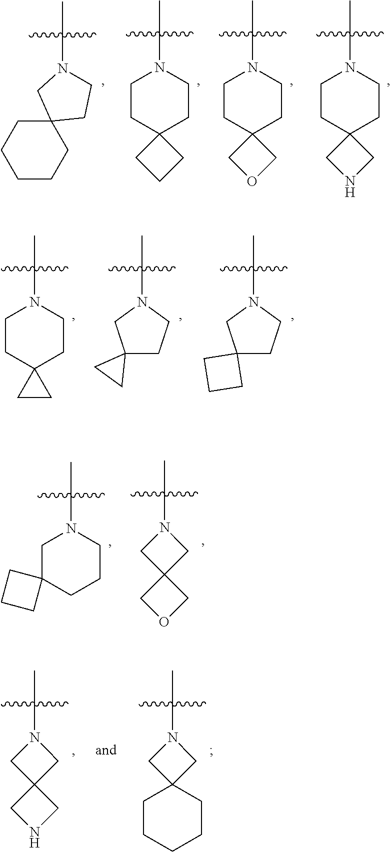

when ring A is

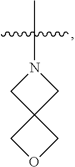 [SMILES: *N1CC2(COC2)C1]

R₂ is not hydrogen.

Further preferably, ring A is selected from

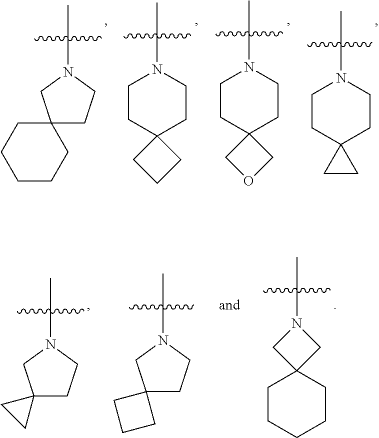

Some embodiments of the present invention relate to a compound of formula (I) or a pharmaceutically acceptable salt or an isomer thereof, wherein

each R₂ is independently selected from hydrogen, cyano, amino, halogen, carboxyl, C₁₋₄ alkyl, C₁₋₄ alkoxy, C₂₋₆ alkenyl, C₁₋₄ alkylcarbonyl, C₂₋₆ alkynyl, C₁₋₄ alkylamino, (C₁₋₄ alkyl)₂amino, C₁₋₄ alkylaminocarbonyl, C₁₋₄ alkylthio, C₁₋₄ alkylsulfonyl, cyclopropyl, azetidinyl, morpholinyl and piperazinyl, wherein the C₁₋₄ alkyl, C₁₋₄ alkoxy, C₂₋₆ alkenyl, C₁₋₄ alkylcarbonyl, C₂₋₆ alkynyl, C₁₋₄ alkylamino, (C₁₋₄ alkyl)₂amino, C₁₋₄ alkylaminocarbonyl, C₁₋₄ alkylthio, C₁₋₄ alkylsulfonyl, cyclopropyl, azetidinyl, morpholinyl and piperazinyl are unsubstituted or optionally substituted with one or more groups independently selected from hydroxy, amino, halogen, C₁₋₄ alkyl, C₁₋₄ alkoxy, C₁₋₄ alkylamino, (C₁₋₄ alkyl)₂amino, cyclopropyl and C₁₋₄ alkylcarbonyloxy;

L is a bond;

ring A is selected from

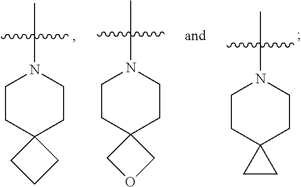

and

m and n are each independently 0, 1 or 2.

Some embodiments of the present invention relate to a compound of formula (I) or a pharmaceutically acceptable salt or an isomer thereof, wherein R₂ is selected from C₁₋₄ alkylaminocarbonyl and (C₁₋₄ alkyl)₂aminocarbonyl;

L is a bond;

ring A is selected from

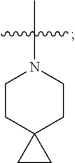 [SMILES: *N1CCC2(CC1)CC2]

m is 0; and

n is 0, 1 or 2.

Some embodiments of the present invention relate to a compound of formula (I) or a pharmaceutically acceptable salt or an isomer thereof, wherein

R₂ is selected from hydrogen, amino, cyano, halogen, carboxyl, C₁₋₄ alkyl, C₁₋₄ alkoxy, C₁₋₄ alkylamino, (C₁₋₄ alkyl)₂amino, C₂₋₆ alkenyl, C₂₋₆ alkynyl, C₁₋₄ alkylcarbonyl, C₁₋₄ alkylaminocarbonyl, (C₁₋₆ alkyl)₂aminocarbonyl, C₁₋₄ alkylsulfonyl, C₁₋₄ alkylthio, aminocarbonyl, cyclopropyl, azetidinyl, morpholinyl and piperazinyl, wherein the C₁₋₄ alkyl, C₁₋₄ alkoxy, C₁₋₄ alkylamino, (C₁₋₄ alkyl)₂amino, C₂₋₆ alkenyl, C₂₋₆ alkynyl, C₁₋₄ alkylcarbonyl, C₁₋₄ alkylaminocarbonyl, (C₁₋₆ alkyl)₂aminocarbonyl, C₁₋₄ alkylsulfonyl, C₁₋₄ alkylthio, aminocarbonyl, cyclopropyl, azetidinyl, morpholinyl and piperazinyl are unsubstituted or optionally substituted with one or more groups independently selected from hydroxy, amino, cyano, halogen, C₁₋₄ alkyl, C₁₋₄ alkoxy, C₁₋₄ alkylamino, (C₁₋₄ alkyl)₂amino, cyclopropyl, C₁₋₄ alkylcarbonyloxy, and 4-6 membered heterocyclyl unsubstituted or optionally substituted with C₁₋₆ alkyl;

L is a bond;

each R₁ is independently selected from hydrogen, hydroxy, cyano, halogen, C₁₋₆ alkyl, C₁₋₆ alkoxy, pyrazolyl, thiazolyl and triazolyl, wherein the C₁₋₆ alkyl, C₁₋₆ alkoxy, pyrazolyl, thiazolyl and triazolyl are unsubstituted or substituted with hydroxy;

m is 0, 1 or 2;

ring A is selected from the following groups:

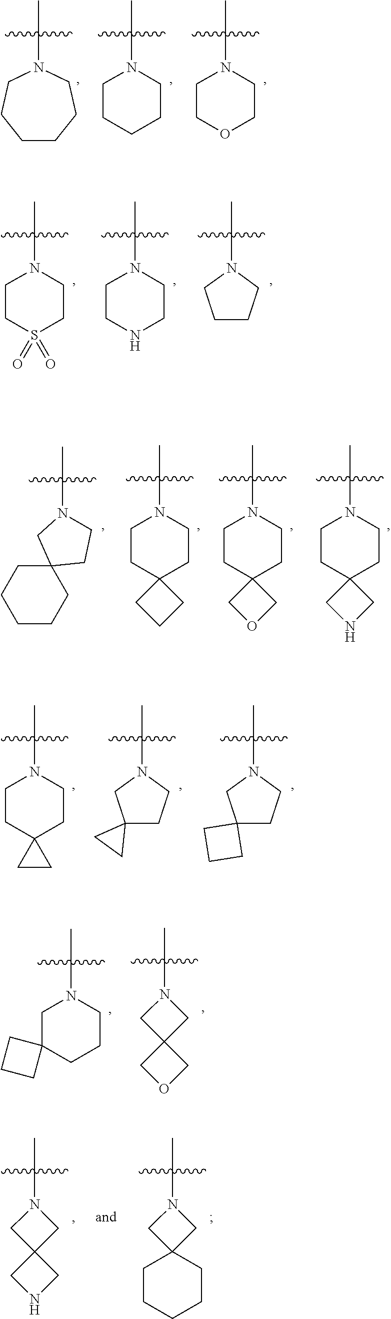

preferably, ring A is selected from

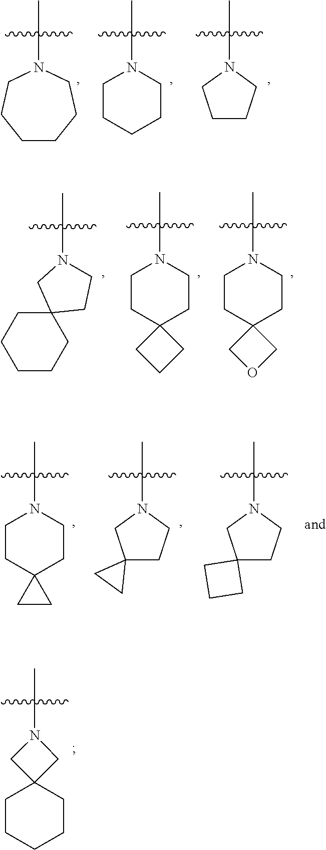

more preferably, ring A is selected from

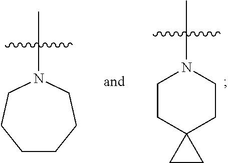 [SMILES: *N1CCCCCC1 ; *N1CCC2(CC1)CC2]

and

when ring A is

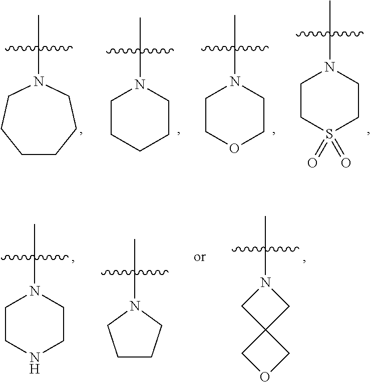

R₂ is not hydrogen.

Some embodiments of the present invention relate to a compound of formula (I) or a pharmaceutically acceptable salt or an isomer thereof, wherein

L is —NH—(CH₂)t- or a bond, wherein t is 0, 1 or 2;

ring A is aryl, and preferably phenyl or naphthyl;

R₂ is selected from hydrogen, amino, carboxyl, cyano, halogen, C₁₋₆ alkyl, C₁₋₆ alkoxy, C₁₋₆ alkylamino, (C₁₋₆ alkyl)₂amino, C₂₋₈ alkenyl, C₂₋₆ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl, wherein the C₁₋₆ alkyl, C₁₋₆ alkoxy, C₁₋₆ alkylamino, (C₁₋₆ alkyl)₂amino, C₂₋₈ alkenyl, C₂₋₆ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl are unsubstituted or optionally substituted with one or more groups independently selected from hydroxy, amino, cyano, halogen, C₁₋₆ alkyl, C₁₋₆ alkoxy, C₁₋₆ alkylamino, (C₁₋₆ alkyl)₂amino, C₁₋₆ alkylcarbonyloxy, C₃₋₆ cycloalkyl, and 4-6 membered heterocyclyl unsubstituted or substituted with C₁₋₆ alkyl;

each R₁ is independently selected from hydrogen, hydroxy, amino, carboxyl, cyano, nitro, halogen, C₁₋₆ alkyl and C₁₋₆ alkoxy, wherein the C₁₋₆ alkyl and C₁₋₆ alkoxy are unsubstituted or optionally substituted with a group selected from hydroxy, amino, carboxyl, cyano, nitro, halogen, C₁₋₆ alkyl and C₁₋₆ alkoxy;

m is 0, 1 or 2; and

when ring A is phenyl, L is not a bond.

Some embodiments of the present invention relate to a compound of formula (I) or a pharmaceutically acceptable salt or an isomer thereof, wherein

L is a bond;

R₂ is selected from hydrogen, amino, carboxyl, cyano, halogen, C₁₋₆ alkyl, C₁₋₆ alkoxy, C₁₋₆ alkylamino, (C₁₋₆ alkyl)₂amino, C₂₋₈ alkenyl, C₂₋₆ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl, wherein the C₁₋₆ alkyl, C₁₋₆ alkoxy, C₁₋₆ alkylamino, (C₁₋₆ alkyl)₂amino, C₂₋₈ alkenyl, C₂₋₆ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl are unsubstituted or optionally substituted with one or more groups independently selected from hydroxy, amino, cyano, halogen, C₁₋₆ alkyl, C₁₋₆ alkoxy, C₁₋₆ alkylamino, (C₁₋₆ alkyl)₂amino, C₁₋₆ alkylcarbonyloxy, C₃₋₆ cycloalkyl, and 4-6 membered heterocyclyl unsubstituted or substituted with C₁₋₆ alkyl;

L is a bond;

ring A is 6-12 membered ortho-fused heterocyclyl, wherein the 6-12 membered ortho-fused heterocyclyl has heteroatoms selected from one of or any combinations of O, S and N;

each R₁ is independently selected from hydrogen, hydroxy, amino, carboxyl, cyano, nitro, halogen, C₁₋₆ alkyl, C₁₋₆ alkoxy, phenyl and 5-6 membered heteroaryl, wherein the C₁₋₆ alkyl, C₁₋₆ alkoxy, phenyl and 5-6 membered heteroaryl are unsubstituted or optionally substituted with a group selected from hydroxy, amino, carboxyl, cyano, nitro, halogen, C₁₋₆ alkyl and C₁₋₆ alkoxy;

m is 0, 1 or 2;

preferably, ring A is 9-10 membered ortho-fused heterocyclyl;

more preferably, ring A is 9-10 membered nitrogen-containing ortho-fused heterocyclyl; and

most preferably, ring A is selected from

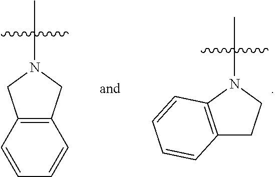 [SMILES: *N1Cc2ccccc2C1 ; *N1CCc2ccccc21]

In one embodiment of the present invention, the isomer of the compound of general formula (I) refers to a stereoisomer or a tautomer.

In one embodiment of the present invention, the compound of general formula (I) has a tautomer, which is shown in general formula (I′).

The tautomer of

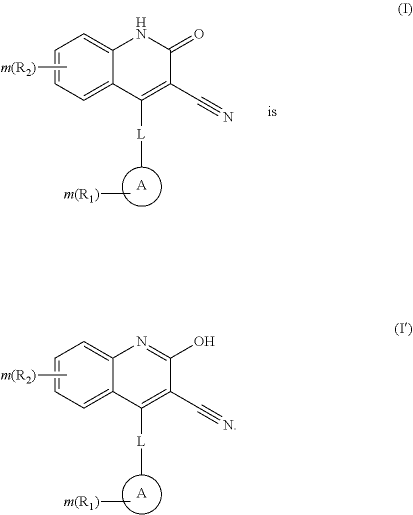

In any of the embodiments of the present invention described above, the compound of formula (I) or the pharmaceutically acceptable salt or the isomer thereof has a structure of general formula (II),

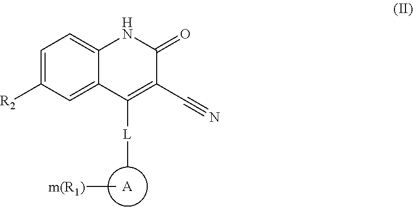

wherein R₁, R₂, L, ring A and m are described as above;

with the proviso that:

when ring A is 3-8 membered monocyclic heterocyclyl, R₂ is not hydrogen;

when ring A is phenyl, L is not a bond; and

when ring A is

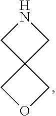

R₂ is not hydrogen.

For the aforementioned compound of formula (I) or (II) or the pharmaceutically acceptable salt or the isomer thereof disclosed herein, in some embodiments, ring A is selected from

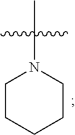 [SMILES: *N1CCCCC1]

in some embodiments, ring A is selected from

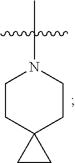 [SMILES: *N1CCC2(CC1)CC2]

in some embodiments, n(R₂) is at position 6 of the quinoline ring in formula (I), as at the position of R₂ in formula (II), and n=1; in some embodiments, R₂ is halogen, C₁₋₄ alkylaminocarbonyl, C₁₋₄ alkyl optionally substituted with one or more groups independently selected from hydroxy and C₃₋₆ cycloalkyl, or C₁₋₄ alkoxy optionally substituted with C₁₋₄ alkoxy. In some embodiments, R₂ is bromine, methyl, ethyl, propyl, isopropyl, isopropyl substituted with hydroxy, methylaminocarbonyl, methyl (or ethyl, or propyl, or butyl) substituted with hydroxy and cyclopropyl, or ethoxy substituted with methoxy. In some embodiments, m(R₁) is para to a site on ring A that is substituted with L; in some embodiments, R₁ is C₁₋₄ alkyl or C₁₋₄ alkoxy; in some embodiments, m=2; and in some embodiments, m=0.

For the aforementioned compound of formula (I) or (II) or the pharmaceutically acceptable salt or the isomer thereof disclosed herein, in some embodiments, ring A is selected from

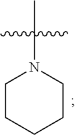 [SMILES: *N1CCCCC1]

m(R₁) is para to a site on ring A that is substituted with L, wherein R₁ is C₁₋₄ alkyl or C₁₋₄ alkoxy, and m=2; and n(R₂) is halogen, C₁₋₄ alkylaminocarbonyl, C₁₋₄ alkyl optionally substituted with one or more groups independently selected from hydroxy and C₃₋₆ cycloalkyl, or C₁₋₄ alkoxy optionally substituted with C₁₋₄ alkoxy, wherein n=1. In some embodiments, ring A is selected from

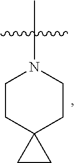 [SMILES: *N1CCC2(CC1)CC2]

and m is 0; and n(R₂) is halogen, C₁₋₄ alkylaminocarbonyl, C₁₋₄ alkyl optionally substituted with one or more groups independently selected from hydroxy and C₃₋₆ cycloalkyl, or C₁₋₄ alkoxy optionally substituted with C₁₋₄ alkoxy, wherein n=1.

For the aforementioned compound of formula (I) or (II) or the pharmaceutically acceptable salt or the isomer thereof disclosed herein, in some embodiments, ring A is selected from

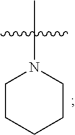 [SMILES: *N1CCCCC1]

m(R₁) is para to a site on ring A that is substituted with L, wherein R₁ is C₁₋₄ alkyl or C₁₋₄ alkoxy, and m=2; and n(R₂) is bromine, methyl, ethyl, propyl, isopropyl, isopropyl substituted with hydroxy, methylaminocarbonyl, methyl (or ethyl, or propyl, or butyl) substituted with hydroxy and cyclopropyl, or ethoxy substituted with methoxy. In some embodiments, ring A is selected from

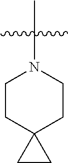 [SMILES: *N1CCC2(CC1)CC2]

and m=0; and n(R₂) is bromine, methyl, ethyl, propyl, isopropyl, isopropyl substituted with hydroxy, methylaminocarbonyl, methyl (or ethyl, or propyl, or butyl) substituted with hydroxy and cyclopropyl, or ethoxy substituted with methoxy.

In one embodiment of the present invention, the aforementioned compounds of formula (I) or (II) or the pharmaceutically acceptable salts or the isomers thereof are shown in Table 1:

TABLE 1

| Serial | |
|---|---|
| number | Structure |
| 1 | 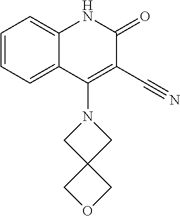 [SMILES: N#Cc1c(N2CC3(COC3)C2)c2ccccc2[nH]c1=O] |
| 2 | 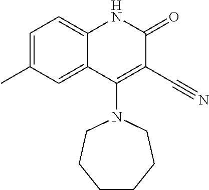 [SMILES: Cc1ccc2[nH]c(=O)c(C#N)c(N3CCCCCC3)c2c1] |
| 3 | 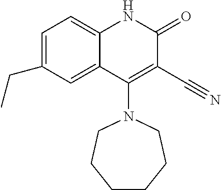 [SMILES: CCc1ccc2[nH]c(=O)c(C#N)c(N3CCCCCC3)c2c1] |
| 4 | 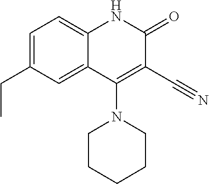 [SMILES: CCc1ccc2[nH]c(=O)c(C#N)c(N3CCCCC3)c2c1] |
| 5 | 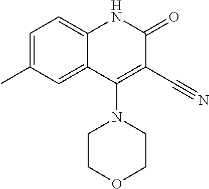 [SMILES: Cc1ccc2[nH]c(=O)c(C#N)c(N3CCOCC3)c2c1] |
| 6 | 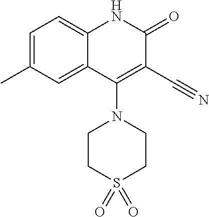 [SMILES: Cc1ccc2[nH]c(=O)c(C#N)c(N3CCS(=O)(=O)CC3)c2c1] |
| 7 | 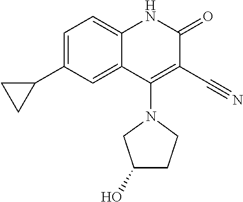 [SMILES: N#Cc1c(N2CC[C@H](O)C2)c2cc(C3CC3)ccc2[nH]c1=O] |
| 8 | 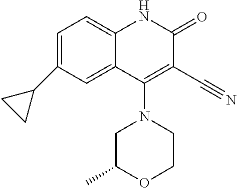 [SMILES: C[C@@H]1CN(c2c(C#N)c(=O)[nH]c3ccc(C4CC4)cc23)CCO1] |
| 9 | 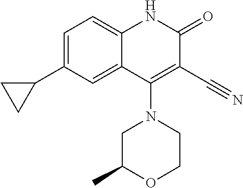 [SMILES: C[C@H]1CN(c2c(C#N)c(=O)[nH]c3ccc(C4CC4)cc23)CCO1] |
| 10 | 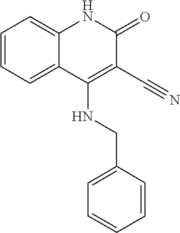 [SMILES: N#Cc1c(NCc2ccccc2)c2ccccc2[nH]c1=O] |
| 11 | 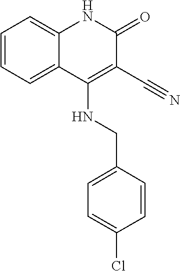 [SMILES: N#Cc1c(NCc2ccc(Cl)cc2)c2ccccc2[nH]c1=O] |
| 12 | 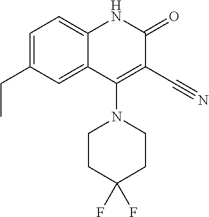 [SMILES: CCc1ccc2[nH]c(=O)c(C#N)c(N3CCC(F)(F)CC3)c2c1] |
| 13 | 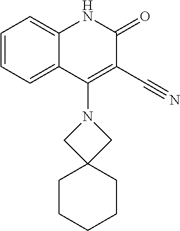 [SMILES: N#Cc1c(N2CC3(CCCCC3)C2)c2ccccc2[nH]c1=O] |
| 14 | 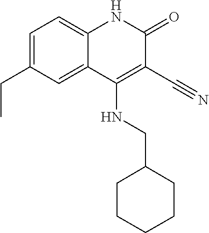 [SMILES: CCc1ccc2[nH]c(=O)c(C#N)c(NCC3CCCCC3)c2c1] |
| 15 | 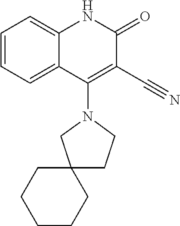 [SMILES: N#Cc1c(N2CCC3(CCCCC3)C2)c2ccccc2[nH]c1=O] |
| 16 | 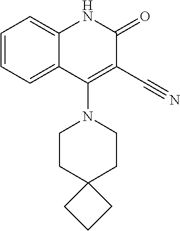 [SMILES: N#Cc1c(N2CCC3(CCC3)CC2)c2ccccc2[nH]c1=O] |
| 17 | 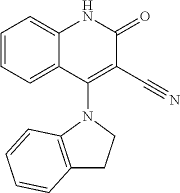 [SMILES: N#Cc1c(N2CCc3ccccc32)c2ccccc2[nH]c1=O] |
| 18 | 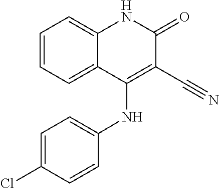 [SMILES: N#Cc1c(Nc2ccc(Cl)cc2)c2ccccc2[nH]c1=O] |
| 19 | 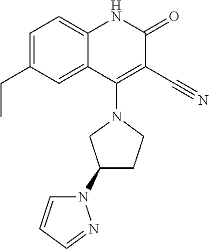 [SMILES: CCc1ccc2[nH]c(=O)c(C#N)c(N3CC[C@@H](n4cccn4)C3)c2c1] |
| 20 | 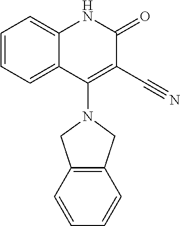 [SMILES: N#Cc1c(N2Cc3ccccc3C2)c2ccccc2[nH]c1=O] |
| 21 | 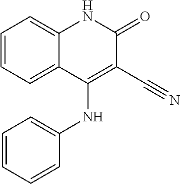 [SMILES: N#Cc1c(Nc2ccccc2)c2ccccc2[nH]c1=O] |
| 22 | 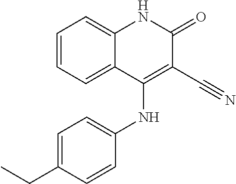 [SMILES: CCc1ccc(Nc2c(C#N)c(=O)[nH]c3ccccc23)cc1] |
| 23 | 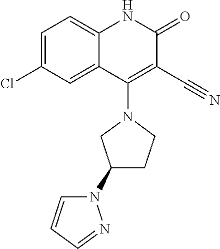 [SMILES: N#Cc1c(N2CC[C@@H](n3cccn3)C2)c2cc(Cl)ccc2[nH]c1=O] |
| 24 | 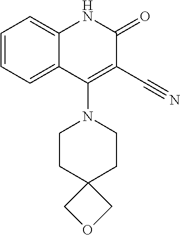 [SMILES: N#Cc1c(N2CCC3(CC2)COC3)c2ccccc2[nH]c1=O] |
| 25 | 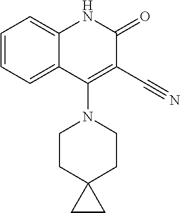 [SMILES: N#Cc1c(N2CCC3(CC2)CC3)c2ccccc2[nH]c1=O] |
| 26 | 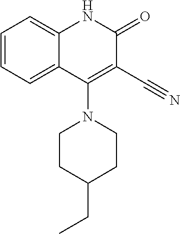 [SMILES: CCC1CCN(c2c(C#N)c(=O)[nH]c3ccccc23)CC1] |
| 27 | 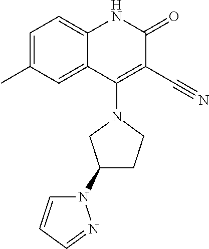 [SMILES: Cc1ccc2[nH]c(=O)c(C#N)c(N3CC[C@@H](n4cccn4)C3)c2c1] |
| 28 | 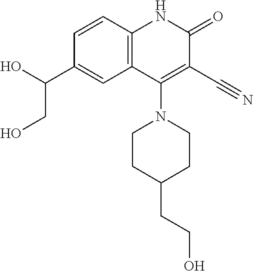 [SMILES: N#Cc1c(N2CCC(CCO)CC2)c2cc(C(O)CO)ccc2[nH]c1=O] |
| 29 | 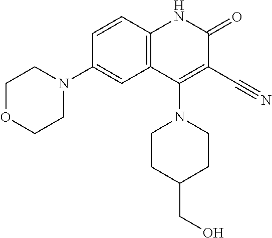 [SMILES: N#Cc1c(N2CCC(CO)CC2)c2cc(N3CCOCC3)ccc2[nH]c1=O] |
| 30 | 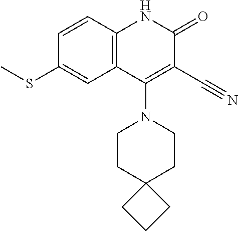 [SMILES: CSc1ccc2[nH]c(=O)c(C#N)c(N3CCC4(CCC4)CC3)c2c1] |
| 31 | 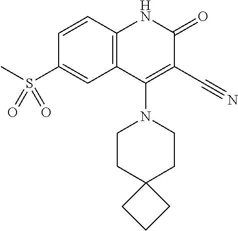 [SMILES: CS(=O)(=O)c1ccc2[nH]c(=O)c(C#N)c(N3CCC4(CCC4)CC3)c2c1] |
| 32 | 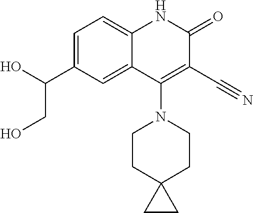 [SMILES: N#Cc1c(N2CCC3(CC2)CC3)c2cc(C(O)CO)ccc2[nH]c1=O] |
| 33 | 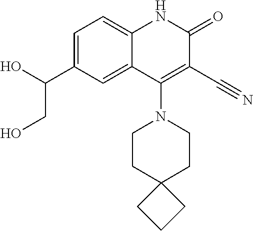 [SMILES: N#Cc1c(N2CCC3(CCC3)CC2)c2cc(C(O)CO)ccc2[nH]c1=O] |
| 34 | 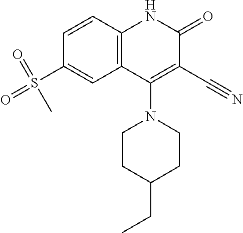 [SMILES: CCC1CCN(c2c(C#N)c(=O)[nH]c3ccc(S(C)(=O)=O)cc23)CC1] |
| 35 | 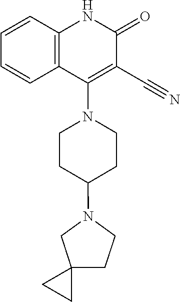 [SMILES: N#Cc1c(N2CCC(N3CCC4(CC4)C3)CC2)c2ccccc2[nH]c1=O] |
| 36 | 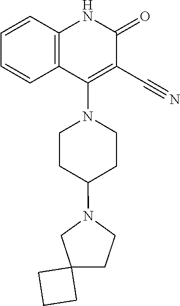 [SMILES: N#Cc1c(N2CCC(N3CCC4(CCC4)C3)CC2)c2ccccc2[nH]c1=O] |
| 37 | 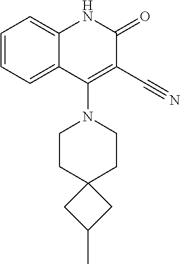 [SMILES: CC1CC2(CCN(c3c(C#N)c(=O)[nH]c4ccccc34)CC2)C1] |
| 38 | 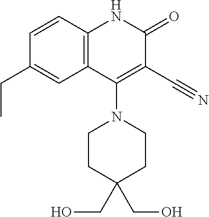 [SMILES: CCc1ccc2[nH]c(=O)c(C#N)c(N3CCC(CO)(CO)CC3)c2c1] |
| 39 | 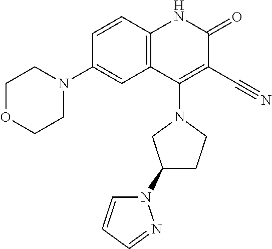 [SMILES: N#Cc1c(N2CC[C@@H](n3cccn3)C2)c2cc(N3CCOCC3)ccc2[nH]c1=O] |
| 40 | 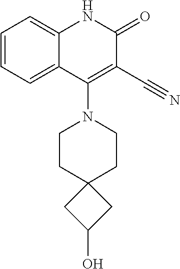 [SMILES: N#Cc1c(N2CCC3(CC2)CC(O)C3)c2ccccc2[nH]c1=O] |
| 41 | 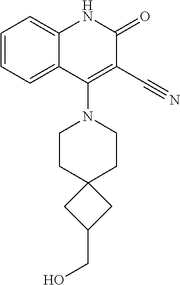 [SMILES: N#Cc1c(N2CCC3(CC2)CC(CO)C3)c2ccccc2[nH]c1=O] |
| 42 | 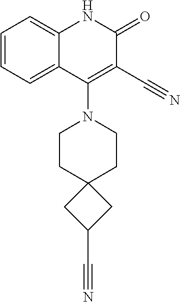 [SMILES: N#Cc1c(N2CCC3(CC2)CC(C#N)C3)c2ccccc2[nH]c1=O] |
| 43 | 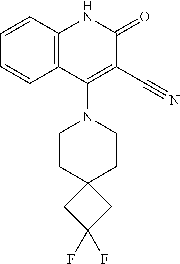 [SMILES: N#Cc1c(N2CCC3(CC2)CC(F)(F)C3)c2ccccc2[nH]c1=O] |
| 44 | 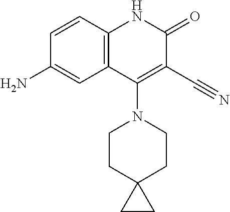 [SMILES: N#Cc1c(N2CCC3(CC2)CC3)c2cc(N)ccc2[nH]c1=O] |
| 45 | 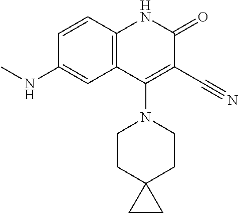 [SMILES: CNc1ccc2[nH]c(=O)c(C#N)c(N3CCC4(CC3)CC4)c2c1] |
| 46 | 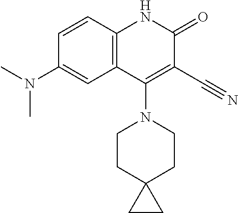 [SMILES: CN(C)c1ccc2[nH]c(=O)c(C#N)c(N3CCC4(CC3)CC4)c2c1] |
| 47 | 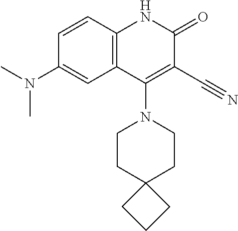 [SMILES: CN(C)c1ccc2[nH]c(=O)c(C#N)c(N3CCC4(CCC4)CC3)c2c1] |
| 48 | 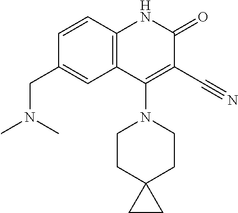 [SMILES: CN(C)Cc1ccc2[nH]c(=O)c(C#N)c(N3CCC4(CC3)CC4)c2c1] |
| 49 | 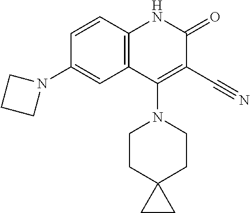 [SMILES: N#Cc1c(N2CCC3(CC2)CC3)c2cc(N3CCC3)ccc2[nH]c1=O] |
| 50 | 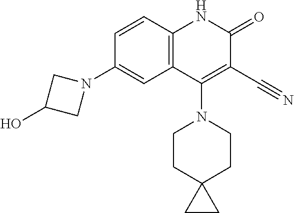 [SMILES: N#Cc1c(N2CCC3(CC2)CC3)c2cc(N3CC(O)C3)ccc2[nH]c1=O] |
| 51 | 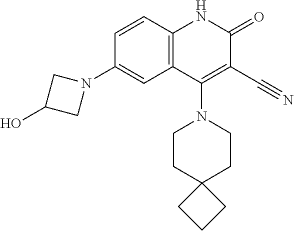 [SMILES: N#Cc1c(N2CCC3(CCC3)CC2)c2cc(N3CC(O)C3)ccc2[nH]c1=O] |
| 52 | 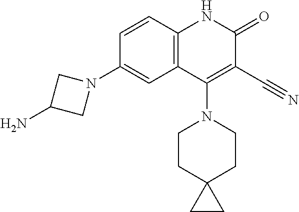 [SMILES: N#Cc1c(N2CCC3(CC2)CC3)c2cc(N3CC(N)C3)ccc2[nH]c1=O] |
| 53 | 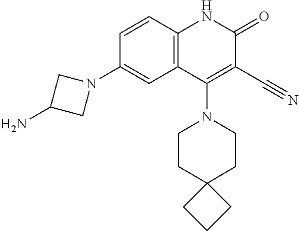 [SMILES: N#Cc1c(N2CCC3(CCC3)CC2)c2cc(N3CC(N)C3)ccc2[nH]c1=O] |
| 54 | 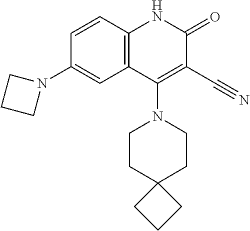 [SMILES: N#Cc1c(N2CCC3(CCC3)CC2)c2cc(N3CCC3)ccc2[nH]c1=O] |
| 55 | 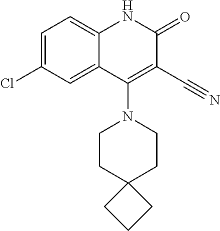 [SMILES: N#Cc1c(N2CCC3(CCC3)CC2)c2cc(Cl)ccc2[nH]c1=O] |
| 56 | 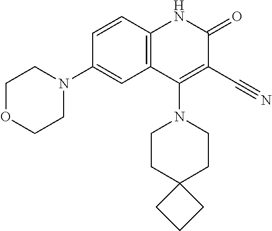 [SMILES: N#Cc1c(N2CCC3(CCC3)CC2)c2cc(N3CCOCC3)ccc2[nH]c1=O] |
| 57 | 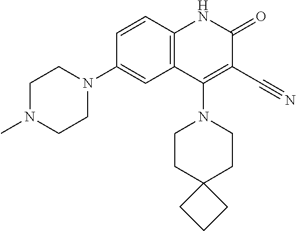 [SMILES: CN1CCN(c2ccc3[nH]c(=O)c(C#N)c(N4CCC5(CCC5)CC4)c3c2)CC1] |
| 58 | 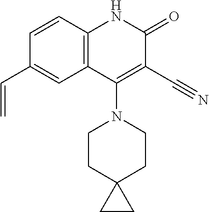 [SMILES: C=Cc1ccc2[nH]c(=O)c(C#N)c(N3CCC4(CC3)CC4)c2c1] |
| 59 | 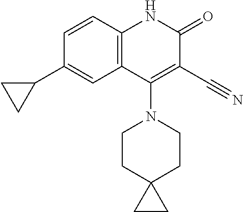 [SMILES: N#Cc1c(N2CCC3(CC2)CC3)c2cc(C3CC3)ccc2[nH]c1=O] |
| 60 | 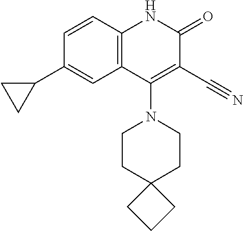 [SMILES: N#Cc1c(N2CCC3(CCC3)CC2)c2cc(C3CC3)ccc2[nH]c1=O] |
| 61 | 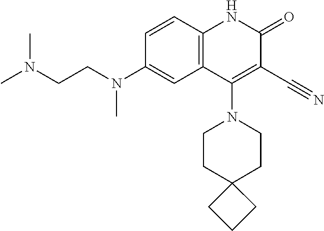 [SMILES: CN(C)CCN(C)c1ccc2[nH]c(=O)c(C#N)c(N3CCC4(CCC4)CC3)c2c1] |
| 62 | 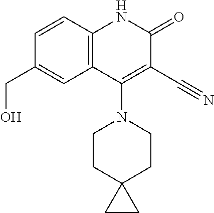 [SMILES: N#Cc1c(N2CCC3(CC2)CC3)c2cc(CO)ccc2[nH]c1=O] |
| 63 | 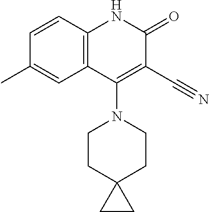 [SMILES: Cc1ccc2[nH]c(=O)c(C#N)c(N3CCC4(CC3)CC4)c2c1] |
| 64 | 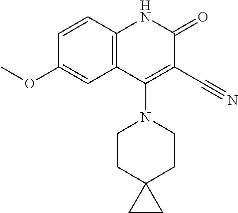 [SMILES: COc1ccc2[nH]c(=O)c(C#N)c(N3CCC4(CC3)CC4)c2c1] |
| 65 | 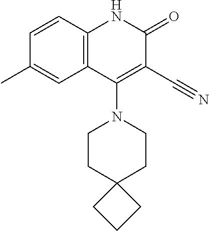 [SMILES: Cc1ccc2[nH]c(=O)c(C#N)c(N3CCC4(CCC4)CC3)c2c1] |
| 66 | 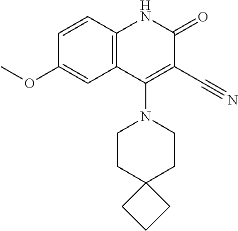 [SMILES: COc1ccc2[nH]c(=O)c(C#N)c(N3CCC4(CCC4)CC3)c2c1] |
| 67 | 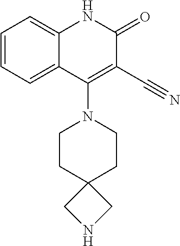 [SMILES: N#Cc1c(N2CCC3(CC2)CNC3)c2ccccc2[nH]c1=O] |
| 68 | 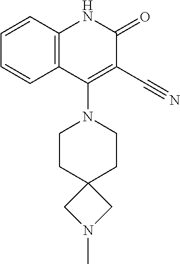 [SMILES: CN1CC2(CCN(c3c(C#N)c(=O)[nH]c4ccccc34)CC2)C1] |
| 69 | 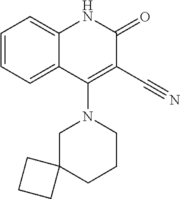 [SMILES: N#Cc1c(N2CCCC3(CCC3)C2)c2ccccc2[nH]c1=O] |
| 70 | 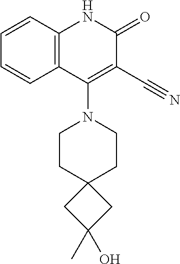 [SMILES: CC1(O)CC2(CCN(c3c(C#N)c(=O)[nH]c4ccccc34)CC2)C1] |
| 71 | 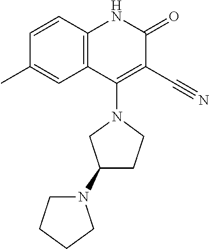 [SMILES: Cc1ccc2[nH]c(=O)c(C#N)c(N3CC[C@@H](N4CCCC4)C3)c2c1] |
| 72 | 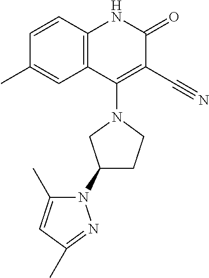 [SMILES: Cc1ccc2[nH]c(=O)c(C#N)c(N3CC[C@@H](n4nc(C)cc4C)C3)c2c1] |
| 73 | 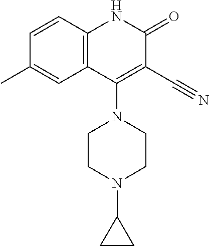 [SMILES: Cc1ccc2[nH]c(=O)c(C#N)c(N3CCN(C4CC4)CC3)c2c1] |
| 74 | 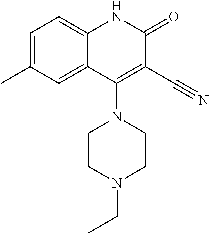 [SMILES: CCN1CCN(c2c(C#N)c(=O)[nH]c3ccc(C)cc23)CC1] |
| 75 | 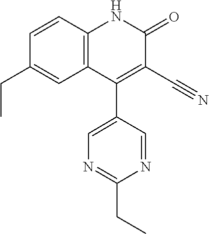 [SMILES: CCc1ccc2[nH]c(=O)c(C#N)c(-c3cnc(CC)nc3)c2c1] |
| 76 | 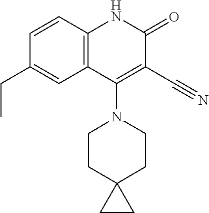 [SMILES: CCc1ccc2[nH]c(=O)c(C#N)c(N3CCC4(CC3)CC4)c2c1] |
| 77 | 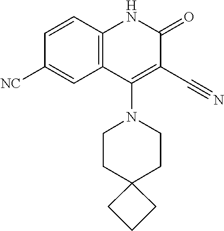 [SMILES: N#Cc1ccc2[nH]c(=O)c(C#N)c(N3CCC4(CCC4)CC3)c2c1] |
| 78 | 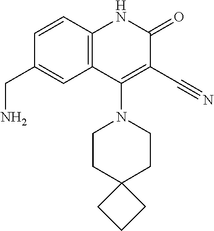 [SMILES: N#Cc1c(N2CCC3(CCC3)CC2)c2cc(CN)ccc2[nH]c1=O] |
| 79 | 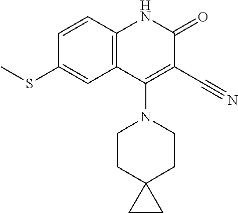 [SMILES: CSc1ccc2[nH]c(=O)c(C#N)c(N3CCC4(CC3)CC4)c2c1] |
| 80 | 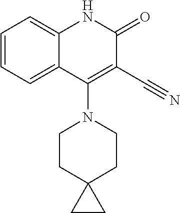 [SMILES: N#Cc1c(N2CCC3(CC2)CC3)c2ccccc2[nH]c1=O] |
| 81 | 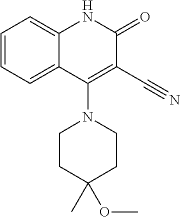 [SMILES: COC1(C)CCN(c2c(C#N)c(=O)[nH]c3ccccc23)CC1] |
| 82 | 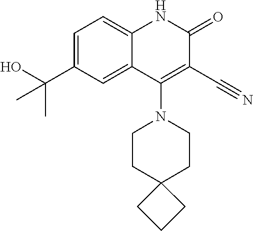 [SMILES: CC(C)(O)c1ccc2[nH]c(=O)c(C#N)c(N3CCC4(CCC4)CC3)c2c1] |
| 83 | 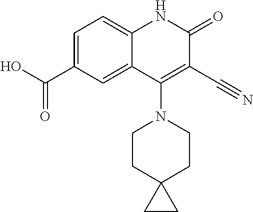 [SMILES: N#Cc1c(N2CCC3(CC2)CC3)c2cc(C(=O)O)ccc2[nH]c1=O] |
| 84 | 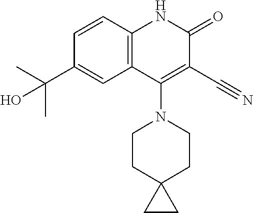 [SMILES: CC(C)(O)c1ccc2[nH]c(=O)c(C#N)c(N3CCC4(CC3)CC4)c2c1] |
| 85 | 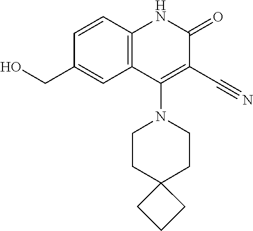 [SMILES: N#Cc1c(N2CCC3(CCC3)CC2)c2cc(CO)ccc2[nH]c1=O] |
| 86 | 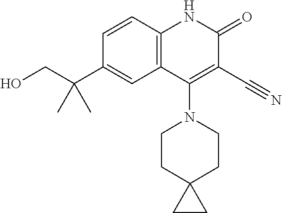 [SMILES: CC(C)(CO)c1ccc2[nH]c(=O)c(C#N)c(N3CCC4(CC3)CC4)c2c1] |
| 87 | 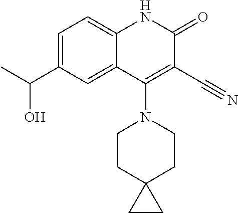 [SMILES: CC(O)c1ccc2[nH]c(=O)c(C#N)c(N3CCC4(CC3)CC4)c2c1] |
| 88 | 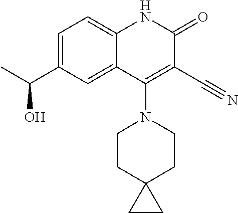 [SMILES: C[C@H](O)c1ccc2[nH]c(=O)c(C#N)c(N3CCC4(CC3)CC4)c2c1] |
| 89 | 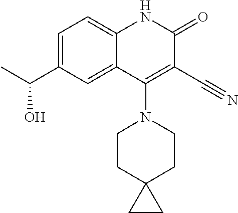 [SMILES: C[C@@H](O)c1ccc2[nH]c(=O)c(C#N)c(N3CCC4(CC3)CC4)c2c1] |
| 90 | 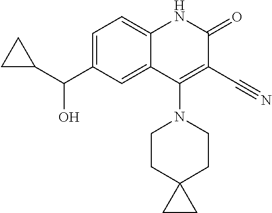 [SMILES: N#Cc1c(N2CCC3(CC2)CC3)c2cc(C(O)C3CC3)ccc2[nH]c1=O] |
| 91 | 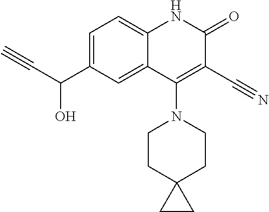 [SMILES: C#CC(O)c1ccc2[nH]c(=O)c(C#N)c(N3CCC4(CC3)CC4)c2c1] |
| 92 | 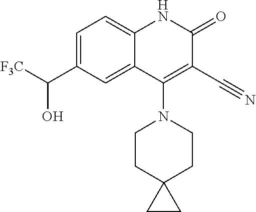 [SMILES: N#Cc1c(N2CCC3(CC2)CC3)c2cc(C(O)C(F)(F)F)ccc2[nH]c1=O] |
| 93 | 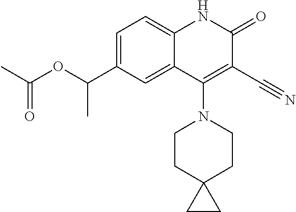 [SMILES: CC(=O)OC(C)c1ccc2[nH]c(=O)c(C#N)c(N3CCC4(CC3)CC4)c2c1] |
| 94 | 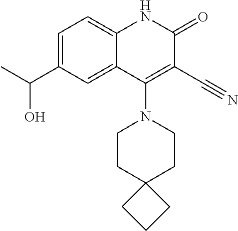 [SMILES: CC(O)c1ccc2[nH]c(=O)c(C#N)c(N3CCC4(CCC4)CC3)c2c1] |
| 95 | 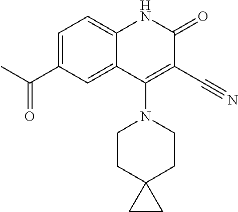 [SMILES: CC(=O)c1ccc2[nH]c(=O)c(C#N)c(N3CCC4(CC3)CC4)c2c1] |
| 96 | 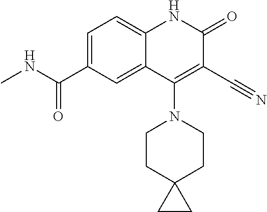 [SMILES: CNC(=O)c1ccc2[nH]c(=O)c(C#N)c(N3CCC4(CC3)CC4)c2c1] |
| 97 | 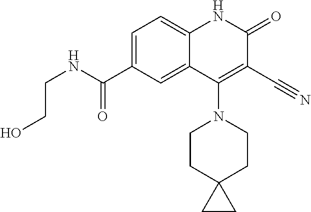 [SMILES: N#Cc1c(N2CCC3(CC2)CC3)c2cc(C(=O)NCCO)ccc2[nH]c1=O] |
| 98 | 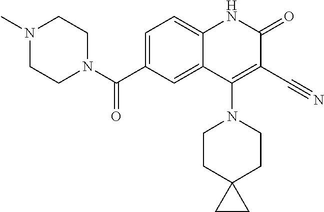 [SMILES: CN1CCN(C(=O)c2ccc3[nH]c(=O)c(C#N)c(N4CCC5(CC4)CC5)c3c2)CC1] |
| 99 | 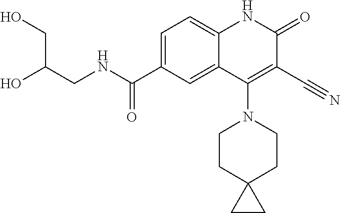 [SMILES: N#Cc1c(N2CCC3(CC2)CC3)c2cc(C(=O)NCC(O)CO)ccc2[nH]c1=O] |
| 100 | 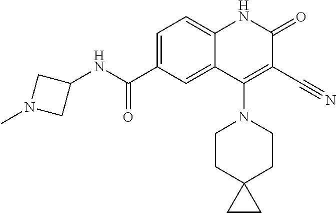 [SMILES: CN1CC(NC(=O)c2ccc3[nH]c(=O)c(C#N)c(N4CCC5(CC4)CC5)c3c2)C1] |
| 101 | 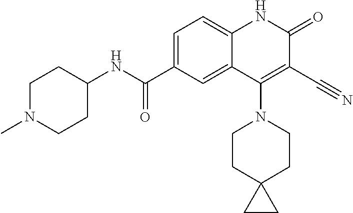 [SMILES: CN1CCC(NC(=O)c2ccc3[nH]c(=O)c(C#N)c(N4CCC5(CC4)CC5)c3c2)CC1] |
| 102 | 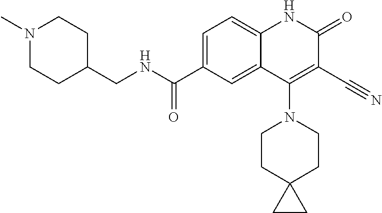 [SMILES: CN1CCC(CNC(=O)c2ccc3[nH]c(=O)c(C#N)c(N4CCC5(CC4)CC5)c3c2)CC1] |
| 103 | 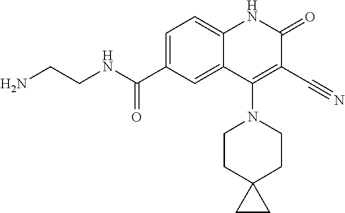 [SMILES: N#Cc1c(N2CCC3(CC2)CC3)c2cc(C(=O)NCCN)ccc2[nH]c1=O] |
| 104 | 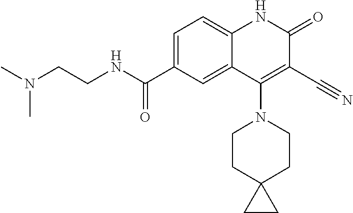 [SMILES: CN(C)CCNC(=O)c1ccc2[nH]c(=O)c(C#N)c(N3CCC4(CC3)CC4)c2c1] |
| 105 | 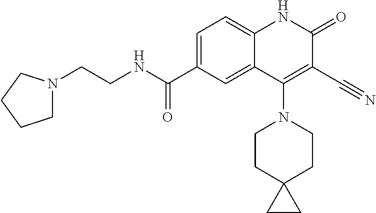 [SMILES: N#Cc1c(N2CCC3(CC2)CC3)c2cc(C(=O)NCCN3CCCC3)ccc2[nH]c1=O] |
| 106 | 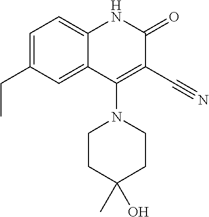 [SMILES: CCc1ccc2[nH]c(=O)c(C#N)c(N3CCC(C)(O)CC3)c2c1] |
| 107 | 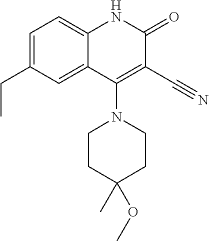 [SMILES: CCc1ccc2[nH]c(=O)c(C#N)c(N3CCC(C)(OC)CC3)c2c1] |
| 108 | 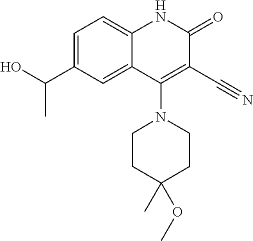 [SMILES: COC1(C)CCN(c2c(C#N)c(=O)[nH]c3ccc(C(C)O)cc23)CC1] |
| 109 | 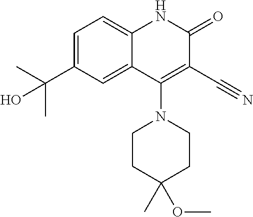 [SMILES: COC1(C)CCN(c2c(C#N)c(=O)[nH]c3ccc(C(C)(C)O)cc23)CC1] |
| 110 | 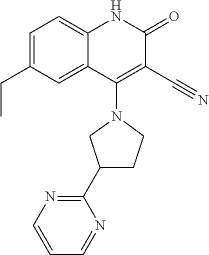 [SMILES: CCc1ccc2[nH]c(=O)c(C#N)c(N3CCC(c4ncccn4)C3)c2c1] |
| 111 | 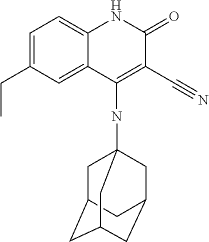 [SMILES: CCc1ccc2[nH]c(=O)c(C#N)c(NC34CC5CC(CC(C5)C3)C4)c2c1] |
| 112 | 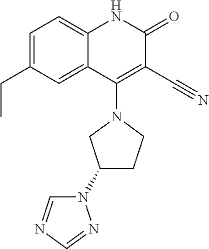 [SMILES: CCc1ccc2[nH]c(=O)c(C#N)c(N3CC[C@H](n4cncn4)C3)c2c1] |
| 113 | 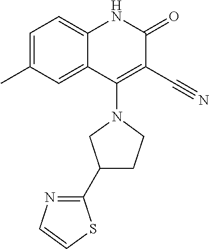 [SMILES: Cc1ccc2[nH]c(=O)c(C#N)c(N3CCC(c4nccs4)C3)c2c1] |
| 114 | 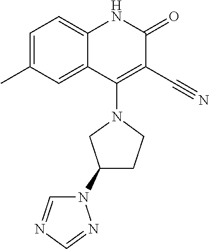 [SMILES: Cc1ccc2[nH]c(=O)c(C#N)c(N3CC[C@@H](n4cncn4)C3)c2c1] |
| 115 | 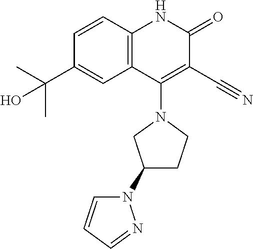 [SMILES: CC(C)(O)c1ccc2[nH]c(=O)c(C#N)c(N3CC[C@@H](n4cccn4)C3)c2c1] |
| 116 | 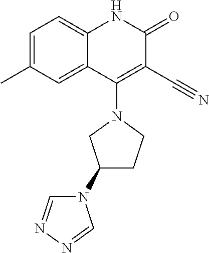 [SMILES: Cc1ccc2[nH]c(=O)c(C#N)c(N3CC[C@@H](n4cnnc4)C3)c2c1] |
| 117 | 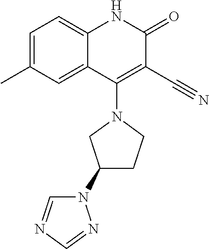 [SMILES: Cc1ccc2[nH]c(=O)c(C#N)c(N3CC[C@@H](n4cncn4)C3)c2c1] |
| 118 | 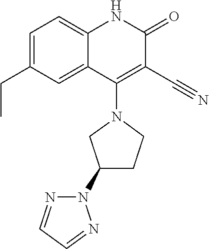 [SMILES: CCc1ccc2[nH]c(=O)c(C#N)c(N3CC[C@@H](n4nccn4)C3)c2c1] |
| 119 | 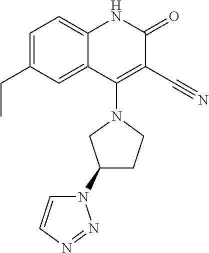 [SMILES: CCc1ccc2[nH]c(=O)c(C#N)c(N3CC[C@@H](n4ccnn4)C3)c2c1] |
| 120 | 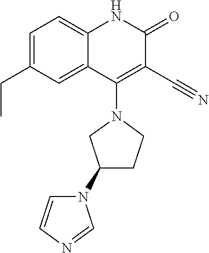 [SMILES: CCc1ccc2[nH]c(=O)c(C#N)c(N3CC[C@@H](n4ccnc4)C3)c2c1] |
| 121 | 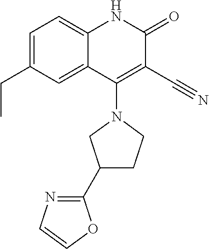 [SMILES: CCc1ccc2[nH]c(=O)c(C#N)c(N3CCC(c4ncco4)C3)c2c1] |
| 122 | 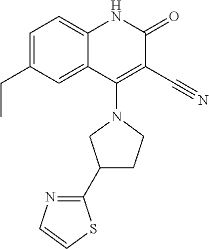 [SMILES: CCc1ccc2[nH]c(=O)c(C#N)c(N3CCC(c4nccs4)C3)c2c1] |
| 123 | 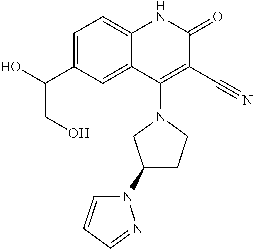 [SMILES: N#Cc1c(N2CC[C@@H](n3cccn3)C2)c2cc(C(O)CO)ccc2[nH]c1=O] |
| 124 | 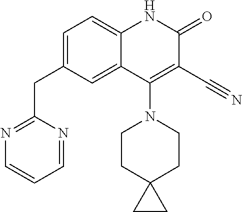 [SMILES: N#Cc1c(N2CCC3(CC2)CC3)c2cc(Cc3ncccn3)ccc2[nH]c1=O] |
| 125 | 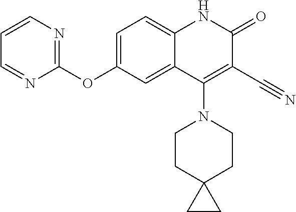 [SMILES: N#Cc1c(N2CCC3(CC2)CC3)c2cc(Oc3ncccn3)ccc2[nH]c1=O] |
| 126 | 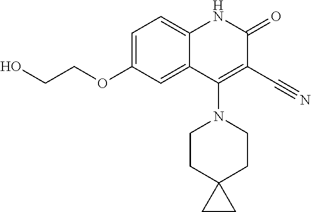 [SMILES: N#Cc1c(N2CCC3(CC2)CC3)c2cc(OCCO)ccc2[nH]c1=O] |
| 127 | 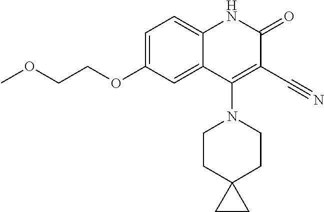 [SMILES: COCCOc1ccc2[nH]c(=O)c(C#N)c(N3CCC4(CC3)CC4)c2c1] |
| 128 | 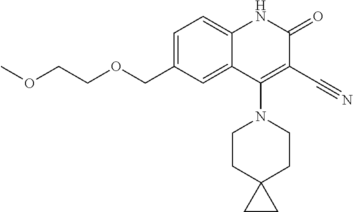 [SMILES: COCCOCc1ccc2[nH]c(=O)c(C#N)c(N3CCC4(CC3)CC4)c2c1] |
| 129 | 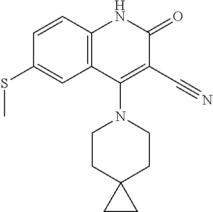 [SMILES: CSc1ccc2[nH]c(=O)c(C#N)c(N3CCC4(CC3)CC4)c2c1] |
| 130 | 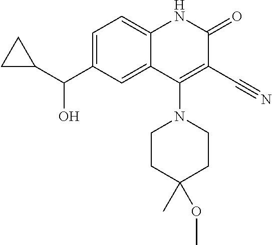 [SMILES: COC1(C)CCN(c2c(C#N)c(=O)[nH]c3ccc(C(O)C4CC4)cc23)CC1] |
| 131 | 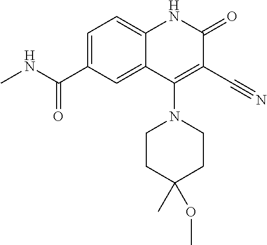 [SMILES: CNC(=O)c1ccc2[nH]c(=O)c(C#N)c(N3CCC(C)(OC)CC3)c2c1] |
| 132 | 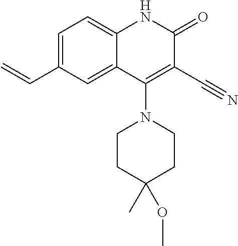 [SMILES: C=Cc1ccc2[nH]c(=O)c(C#N)c(N3CCC(C)(OC)CC3)c2c1] |
| 133 | 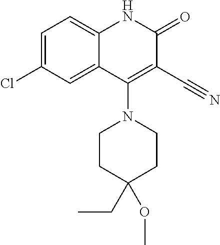 [SMILES: CCC1(OC)CCN(c2c(C#N)c(=O)[nH]c3ccc(Cl)cc23)CC1] |
| 134 | 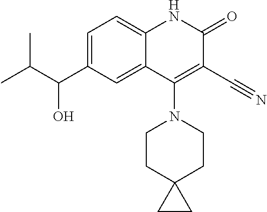 [SMILES: CC(C)C(O)c1ccc2[nH]c(=O)c(C#N)c(N3CCC4(CC3)CC4)c2c1] |
| 135 | 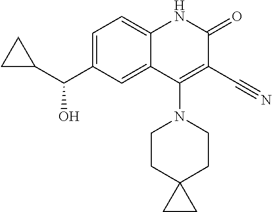 [SMILES: N#Cc1c(N2CCC3(CC2)CC3)c2cc([C@H](O)C3CC3)ccc2[nH]c1=O] |
| 136 | 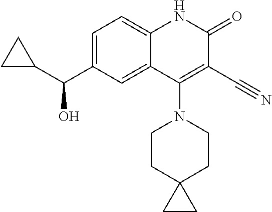 [SMILES: N#Cc1c(N2CCC3(CC2)CC3)c2cc([C@@H](O)C3CC3)ccc2[nH]c1=O] |
| 137 | 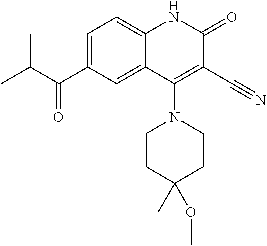 [SMILES: COC1(C)CCN(c2c(C#N)c(=O)[nH]c3ccc(C(=O)C(C)C)cc23)CC1] |
| 138 | 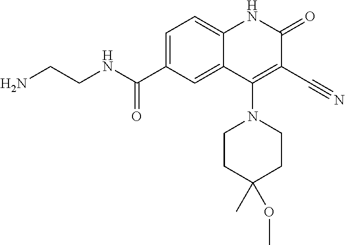 [SMILES: COC1(C)CCN(c2c(C#N)c(=O)[nH]c3ccc(C(=O)NCCN)cc23)CC1] |
| 139 | 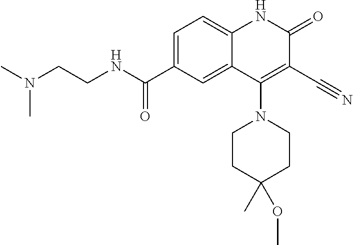 [SMILES: COC1(C)CCN(c2c(C#N)c(=O)[nH]c3ccc(C(=O)NCCN(C)C)cc23)CC1] |
| 140 | 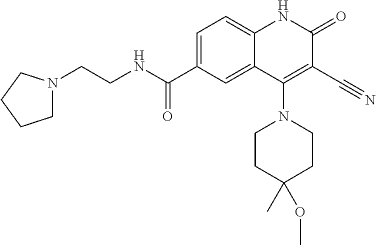 [SMILES: COC1(C)CCN(c2c(C#N)c(=O)[nH]c3ccc(C(=O)NCCN4CCCC4)cc23)CC1] |
| 141 | 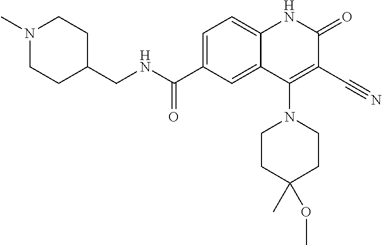 [SMILES: COC1(C)CCN(c2c(C#N)c(=O)[nH]c3ccc(C(=O)NCC4CCN(C)CC4)cc23)CC1] |
| 142 | 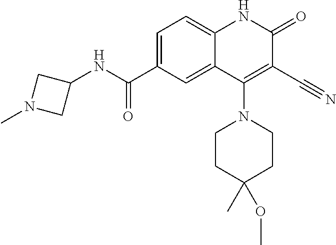 [SMILES: COC1(C)CCN(c2c(C#N)c(=O)[nH]c3ccc(C(=O)NC4CN(C)C4)cc23)CC1] |
| 143 | 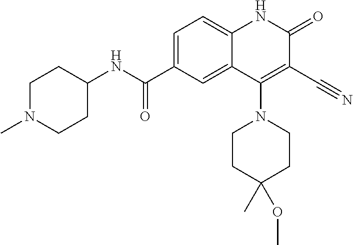 [SMILES: COC1(C)CCN(c2c(C#N)c(=O)[nH]c3ccc(C(=O)NC4CCN(C)CC4)cc23)CC1] |
| 144 | 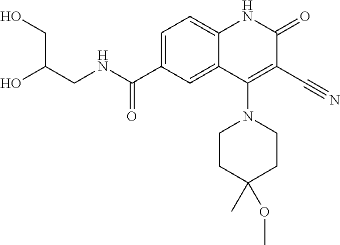 [SMILES: COC1(C)CCN(c2c(C#N)c(=O)[nH]c3ccc(C(=O)NCC(O)CO)cc23)CC1] |
| 145 | 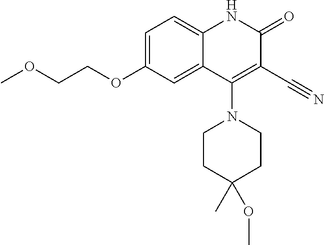 [SMILES: COCCOc1ccc2[nH]c(=O)c(C#N)c(N3CCC(C)(OC)CC3)c2c1] |
| 146 | 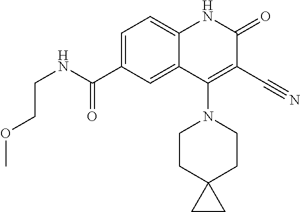 [SMILES: COCCNC(=O)c1ccc2[nH]c(=O)c(C#N)c(N3CCC4(CC3)CC4)c2c1] |
| 147 | 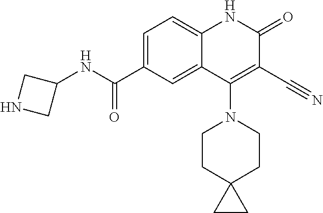 [SMILES: N#Cc1c(N2CCC3(CC2)CC3)c2cc(C(=O)NC3CNC3)ccc2[nH]c1=O] |
| 148 | 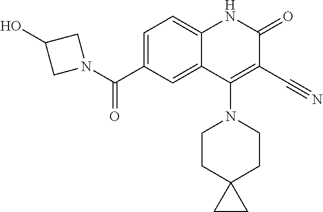 [SMILES: N#Cc1c(N2CCC3(CC2)CC3)c2cc(C(=O)N3CC(O)C3)ccc2[nH]c1=O] |
| 149 | 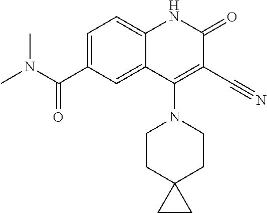 [SMILES: CN(C)C(=O)c1ccc2[nH]c(=O)c(C#N)c(N3CCC4(CC3)CC4)c2c1] |
| 150 | 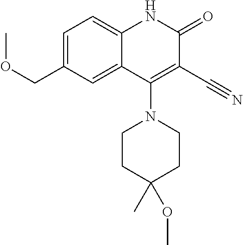 [SMILES: COCc1ccc2[nH]c(=O)c(C#N)c(N3CCC(C)(OC)CC3)c2c1] |
| 151 | 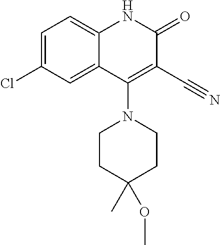 [SMILES: COC1(C)CCN(c2c(C#N)c(=O)[nH]c3ccc(Cl)cc23)CC1] |
| 152 | 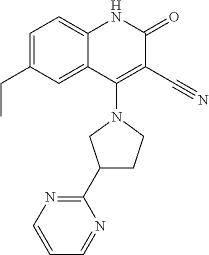 [SMILES: CCc1ccc2[nH]c(=O)c(C#N)c(N3CCC(c4ncccn4)C3)c2c1] |
| 153 | 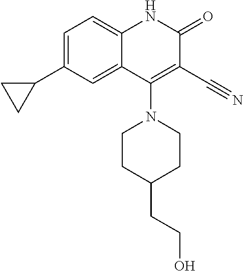 [SMILES: N#Cc1c(N2CCC(CCO)CC2)c2cc(C3CC3)ccc2[nH]c1=O] |
| 154 | 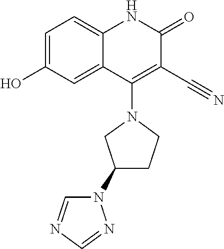 [SMILES: N#Cc1c(N2CC[C@@H](n3cncn3)C2)c2cc(O)ccc2[nH]c1=O] |
| 155 | 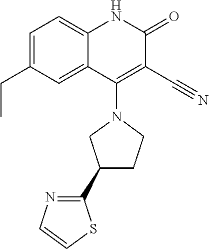 [SMILES: CCc1ccc2[nH]c(=O)c(C#N)c(N3CC[C@@H](c4nccs4)C3)c2c1] |
| 156 | 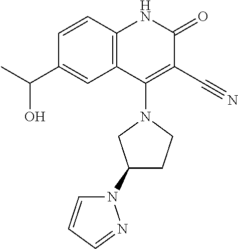 [SMILES: CC(O)c1ccc2[nH]c(=O)c(C#N)c(N3CC[C@@H](n4cccn4)C3)c2c1] |
| 157 | 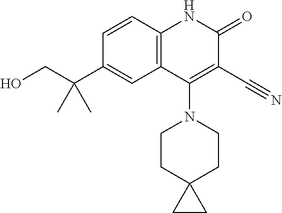 [SMILES: CC(C)(CO)c1ccc2[nH]c(=O)c(C#N)c(N3CCC4(CC3)CC4)c2c1] |
| 158 | 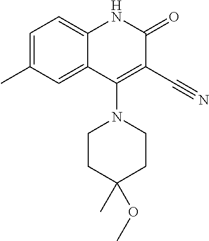 [SMILES: COC1(C)CCN(c2c(C#N)c(=O)[nH]c3ccc(C)cc23)CC1] |
| 159 | 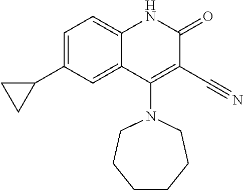 [SMILES: N#Cc1c(N2CCCCCC2)c2cc(C3CC3)ccc2[nH]c1=O] |
| 160 | 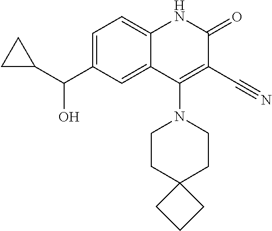 [SMILES: N#Cc1c(N2CCC3(CCC3)CC2)c2cc(C(O)C3CC3)ccc2[nH]c1=O] |
| 161 | 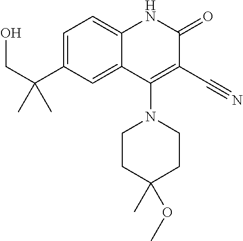 [SMILES: COC1(C)CCN(c2c(C#N)c(=O)[nH]c3ccc(C(C)(C)CO)cc23)CC1] |
| 162 | 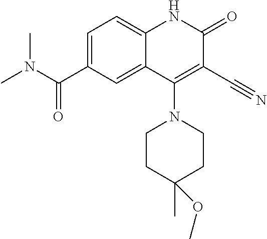 [SMILES: COC1(C)CCN(c2c(C#N)c(=O)[nH]c3ccc(C(=O)N(C)C)cc23)CC1] |
| 163 | 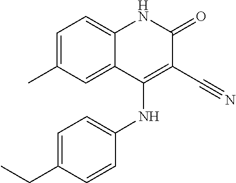 [SMILES: CCc1ccc(Nc2c(C#N)c(=O)[nH]c3ccc(C)cc23)cc1] |
| 164 | 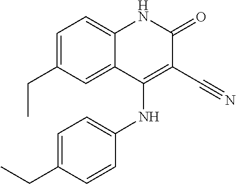 [SMILES: CCc1ccc(Nc2c(C#N)c(=O)[nH]c3ccc(CC)cc23)cc1] |
| 165 | 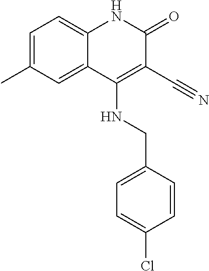 [SMILES: Cc1ccc2[nH]c(=O)c(C#N)c(NCc3ccc(Cl)cc3)c2c1] |
| 166 | 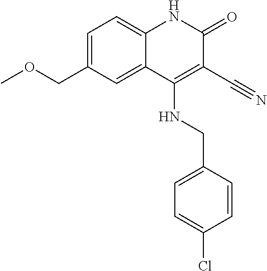 [SMILES: COCc1ccc2[nH]c(=O)c(C#N)c(NCc3ccc(Cl)cc3)c2c1] |
| 167 | 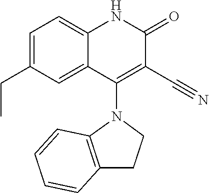 [SMILES: CCc1ccc2[nH]c(=O)c(C#N)c(N3CCc4ccccc43)c2c1] |
| 168 | 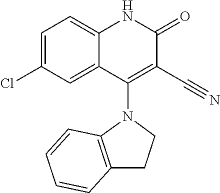 [SMILES: N#Cc1c(N2CCc3ccccc32)c2cc(Cl)ccc2[nH]c1=O] |
| 169 | 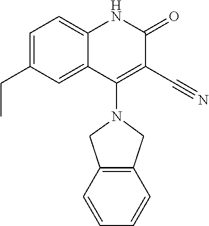 [SMILES: CCc1ccc2[nH]c(=O)c(C#N)c(N3Cc4ccccc4C3)c2c1] |
| 170 | 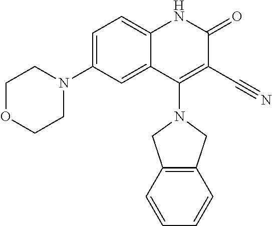 [SMILES: N#Cc1c(N2Cc3ccccc3C2)c2cc(N3CCOCC3)ccc2[nH]c1=O] |
| 171 | 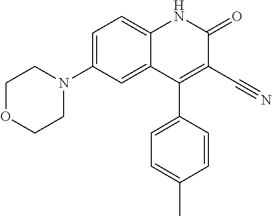 [SMILES: Cc1ccc(-c2c(C#N)c(=O)[nH]c3ccc(N4CCOCC4)cc23)cc1] |
| 172 | 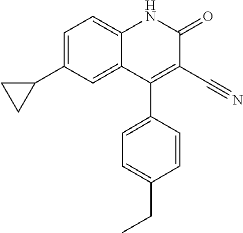 [SMILES: CCc1ccc(-c2c(C#N)c(=O)[nH]c3ccc(C4CC4)cc23)cc1] |
| 173 | 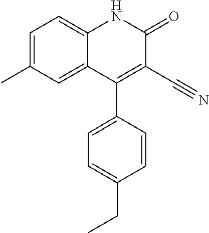 [SMILES: CCc1ccc(-c2c(C#N)c(=O)[nH]c3ccc(C)cc23)cc1] |
| 174 | 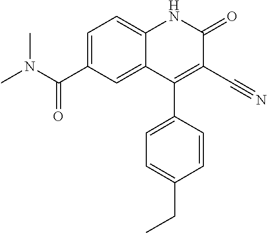 [SMILES: CCc1ccc(-c2c(C#N)c(=O)[nH]c3ccc(C(=O)N(C)C)cc23)cc1] |
| 175 | 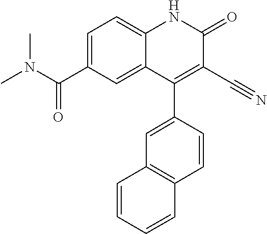 [SMILES: CN(C)C(=O)c1ccc2[nH]c(=O)c(C#N)c(-c3ccc4ccccc4c3)c2c1] |
| 176 | 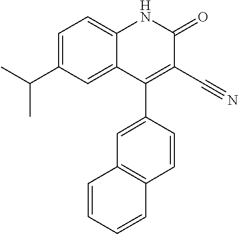 [SMILES: CC(C)c1ccc2[nH]c(=O)c(C#N)c(-c3ccc4ccccc4c3)c2c1] |
| 177 | 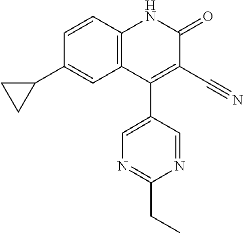 [SMILES: CCc1ncc(-c2c(C#N)c(=O)[nH]c3ccc(C4CC4)cc23)cn1] |
| 178 | 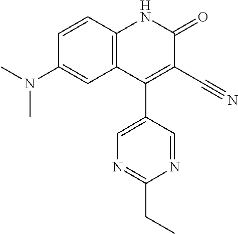 [SMILES: CCc1ncc(-c2c(C#N)c(=O)[nH]c3ccc(N(C)C)cc23)cn1] |
| 179 | 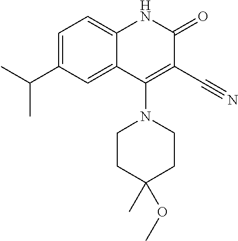 [SMILES: COC1(C)CCN(c2c(C#N)c(=O)[nH]c3ccc(C(C)C)cc23)CC1] |
| 180 | 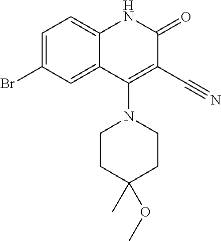 [SMILES: COC1(C)CCN(c2c(C#N)c(=O)[nH]c3ccc(Br)cc23)CC1] |
| 181 | 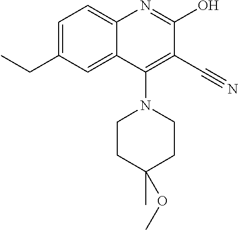 [SMILES: CCc1ccc2nc(O)c(C#N)c(N3CCC(C)(OC)CC3)c2c1] |
| 182 | 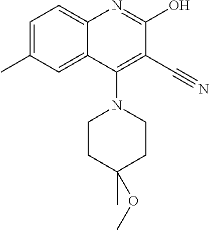 [SMILES: COC1(C)CCN(c2c(C#N)c(O)nc3ccc(C)cc23)CC1] |
| 183 | 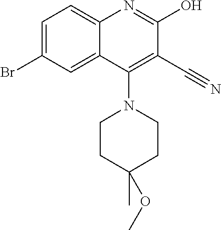 [SMILES: COC1(C)CCN(c2c(C#N)c(O)nc3ccc(Br)cc23)CC1] |
| 184 | 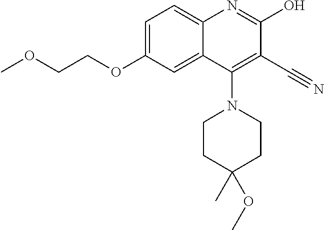 [SMILES: COCCOc1ccc2nc(O)c(C#N)c(N3CCC(C)(OC)CC3)c2c1] |
| 185 | 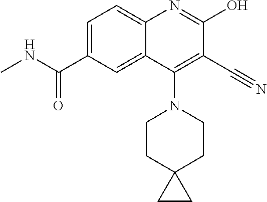 [SMILES: CNC(=O)c1ccc2nc(O)c(C#N)c(N3CCC4(CC3)CC4)c2c1] |
| 186 | 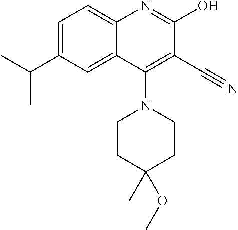 [SMILES: COC1(C)CCN(c2c(C#N)c(O)nc3ccc(C(C)C)cc23)CC1] |
| 187 | 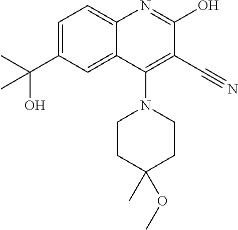 [SMILES: COC1(C)CCN(c2c(C#N)c(O)nc3ccc(C(C)(C)O)cc23)CC1] |
| 188 | 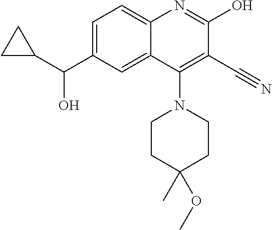 [SMILES: COC1(C)CCN(c2c(C#N)c(O)nc3ccc(C(O)C4CC4)cc23)CC1] |
| 189 | 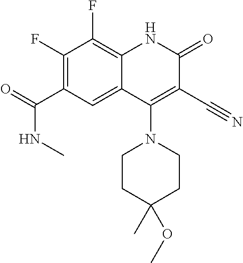 [SMILES: CNC(=O)c1cc2c(N3CCC(C)(OC)CC3)c(C#N)c(=O)[nH]c2c(F)c1F] |
| 190 | 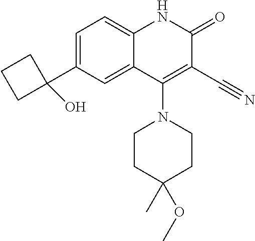 [SMILES: COC1(C)CCN(c2c(C#N)c(=O)[nH]c3ccc(C4(O)CCC4)cc23)CC1] |
| 191 | 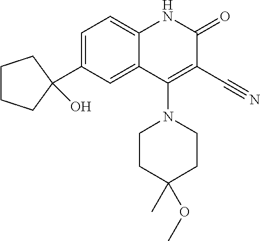 [SMILES: COC1(C)CCN(c2c(C#N)c(=O)[nH]c3ccc(C4(O)CCCC4)cc23)CC1] |
| 192 | 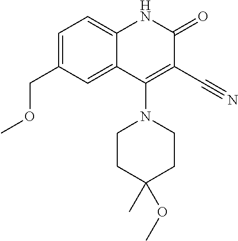 [SMILES: COCc1ccc2[nH]c(=O)c(C#N)c(N3CCC(C)(OC)CC3)c2c1] |
| 193 | 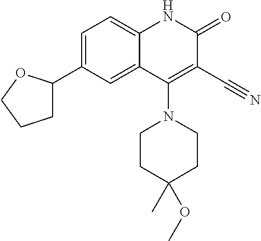 [SMILES: COC1(C)CCN(c2c(C#N)c(=O)[nH]c3ccc(C4CCCO4)cc23)CC1] |
| 194 | 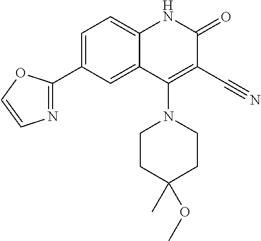 [SMILES: COC1(C)CCN(c2c(C#N)c(=O)[nH]c3ccc(-c4ncco4)cc23)CC1] |
| 195 | 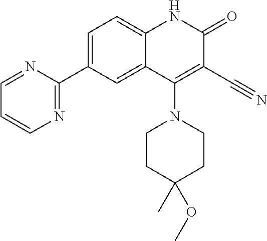 [SMILES: COC1(C)CCN(c2c(C#N)c(=O)[nH]c3ccc(-c4ncccn4)cc23)CC1] |
| 196 | 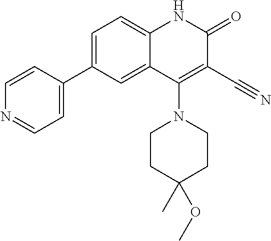 [SMILES: COC1(C)CCN(c2c(C#N)c(=O)[nH]c3ccc(-c4ccncc4)cc23)CC1] |
| 197 | 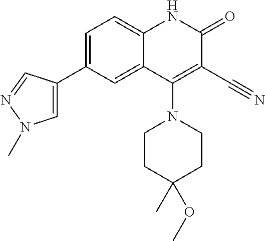 [SMILES: COC1(C)CCN(c2c(C#N)c(=O)[nH]c3ccc(-c4cnn(C)c4)cc23)CC1] |
| 198 | 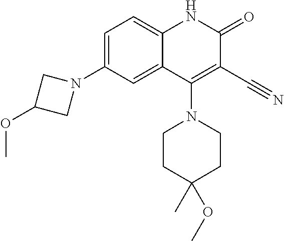 [SMILES: COC1CN(c2ccc3[nH]c(=O)c(C#N)c(N4CCC(C)(OC)CC4)c3c2)C1] |
| 199 | 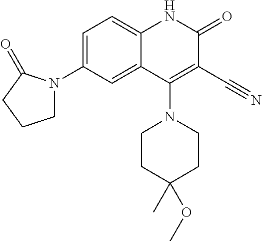 [SMILES: COC1(C)CCN(c2c(C#N)c(=O)[nH]c3ccc(N4CCCC4=O)cc23)CC1] |
| 200 | 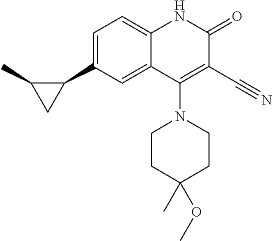 [SMILES: COC1(C)CCN(c2c(C#N)c(=O)[nH]c3ccc([C@H]4C[C@H]4C)cc23)CC1] |
| 201 | 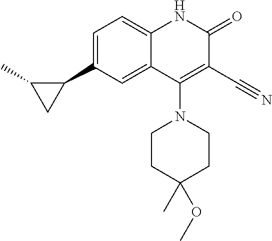 [SMILES: COC1(C)CCN(c2c(C#N)c(=O)[nH]c3ccc([C@H]4C[C@@H]4C)cc23)CC1] |
| 202 | 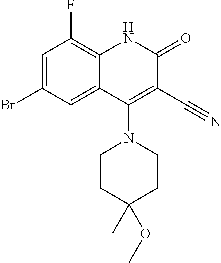 [SMILES: COC1(C)CCN(c2c(C#N)c(=O)[nH]c3c(F)cc(Br)cc23)CC1] |
| 203 | 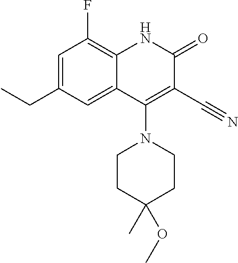 [SMILES: CCc1cc(F)c2[nH]c(=O)c(C#N)c(N3CCC(C)(OC)CC3)c2c1] |
| 204 | 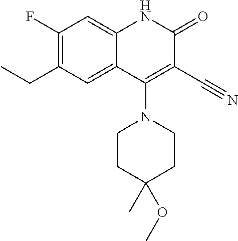 [SMILES: CCc1cc2c(N3CCC(C)(OC)CC3)c(C#N)c(=O)[nH]c2cc1F] |
| 205 | 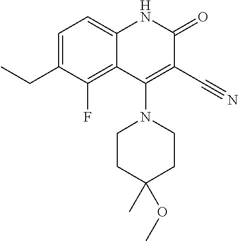 [SMILES: CCc1ccc2[nH]c(=O)c(C#N)c(N3CCC(C)(OC)CC3)c2c1F] |
| 206 | 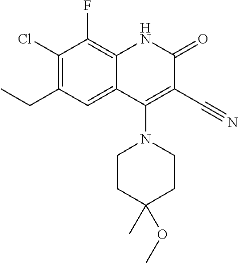 [SMILES: CCc1cc2c(N3CCC(C)(OC)CC3)c(C#N)c(=O)[nH]c2c(F)c1Cl] |
| 207 | 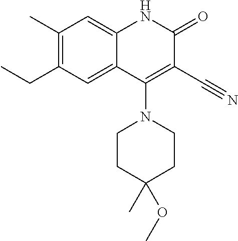 [SMILES: CCc1cc2c(N3CCC(C)(OC)CC3)c(C#N)c(=O)[nH]c2cc1C] |
| 208 | 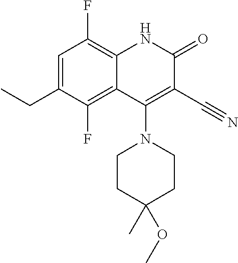 [SMILES: CCc1cc(F)c2[nH]c(=O)c(C#N)c(N3CCC(C)(OC)CC3)c2c1F] |
| 209 | 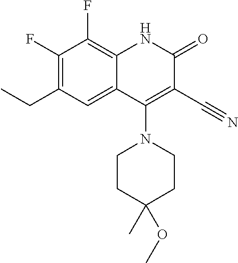 [SMILES: CCc1cc2c(N3CCC(C)(OC)CC3)c(C#N)c(=O)[nH]c2c(F)c1F] |
| 210 | 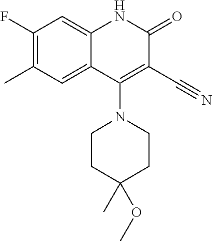 [SMILES: COC1(C)CCN(c2c(C#N)c(=O)[nH]c3cc(F)c(C)cc23)CC1] |
| 211 | 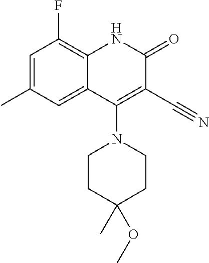 [SMILES: COC1(C)CCN(c2c(C#N)c(=O)[nH]c3c(F)cc(C)cc23)CC1] |
| 212 | 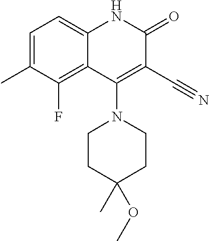 [SMILES: COC1(C)CCN(c2c(C#N)c(=O)[nH]c3ccc(C)c(F)c23)CC1] |
| 213 | 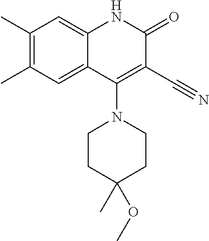 [SMILES: COC1(C)CCN(c2c(C#N)c(=O)[nH]c3cc(C)c(C)cc23)CC1] |
| 214 | 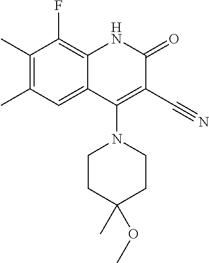 [SMILES: COC1(C)CCN(c2c(C#N)c(=O)[nH]c3c(F)c(C)c(C)cc23)CC1] |
| 215 | 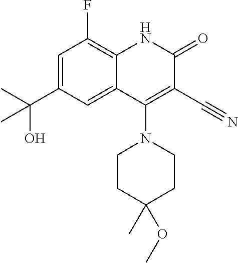 [SMILES: COC1(C)CCN(c2c(C#N)c(=O)[nH]c3c(F)cc(C(C)(C)O)cc23)CC1] |
| 216 | 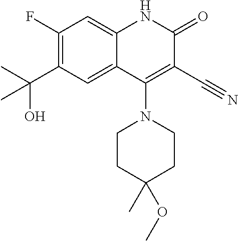 [SMILES: COC1(C)CCN(c2c(C#N)c(=O)[nH]c3cc(F)c(C(C)(C)O)cc23)CC1] |
| 217 | 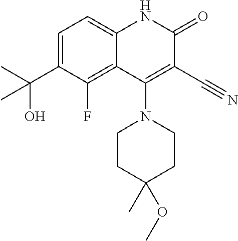 [SMILES: COC1(C)CCN(c2c(C#N)c(=O)[nH]c3ccc(C(C)(C)O)c(F)c23)CC1] |
| 218 | 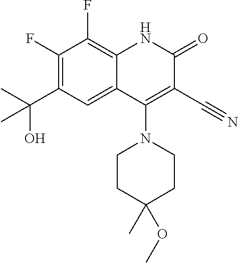 [SMILES: COC1(C)CCN(c2c(C#N)c(=O)[nH]c3c(F)c(F)c(C(C)(C)O)cc23)CC1] |
| 219 | 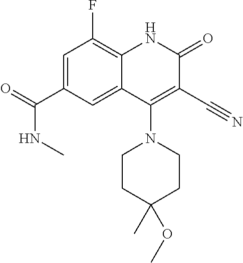 [SMILES: CNC(=O)c1cc(F)c2[nH]c(=O)c(C#N)c(N3CCC(C)(OC)CC3)c2c1] |
| 220 | 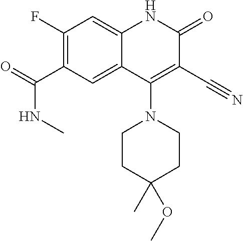 [SMILES: CNC(=O)c1cc2c(N3CCC(C)(OC)CC3)c(C#N)c(=O)[nH]c2cc1F] |
| 221 | 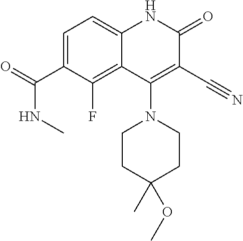 [SMILES: CNC(=O)c1ccc2[nH]c(=O)c(C#N)c(N3CCC(C)(OC)CC3)c2c1F] |
| 222 | 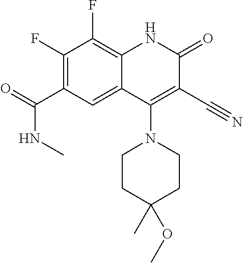 [SMILES: CNC(=O)c1cc2c(N3CCC(C)(OC)CC3)c(C#N)c(=O)[nH]c2c(F)c1F] |

The present invention further provides a pharmaceutical composition comprising the aforementioned compound of formula (I) or (II) or the pharmaceutically acceptable salt or the isomer thereof, and one or more second therapeutic active agents.

In one embodiment of the present invention, the composition may be used by administering a “therapeutically effective amount” of the aforementioned compound of formula (I) or formula (II) or the pharmaceutically acceptable salt or the isomer thereof and one or more second therapeutically active agents in combination, for example, sequential administration, simultaneous administration, or administration in a form of a combination formulation comprising the compound or the pharmaceutically acceptable salts or the isomers thereof provided herein and second therapeutically active agents.

The second therapeutically active agent is selected from acetylcholinesterase inhibitors, amyloid-β (or fragments thereof), antibodies of amyloid-β (or fragments thereof), amyloid-lowering or -inhibiting agents, α-adrenoceptor antagonists, β-adrenoceptor blockers, anticholinergics, anticonvulsants, tranquilizers, calcium channel blockers, catechol-O-methyltransferase inhibitors, central nervous system stimulators, corticosteroids, dopamine receptor agonists, dopamine receptor antagonists, dopamine reuptake inhibitors, γ-aminobutyric acid receptor agonists, immunomodulators, immunosuppressants, interferons, levodopa, N-methyl-D-aspartate receptor antagonists, monoamine oxidase inhibitors, muscarinic receptor agonists, nicotinic receptor agonists, neuroprotective agents, norepinephrine reuptake inhibitors, other PDE9 inhibitors, other phosphodiesterase (PDE) inhibitors, β-secretase inhibitors, γ-secretase inhibitors, serotonin (5-hydroxytryptamine)1A (5-HT_{1A}) receptor antagonists, serotonin (5-hydroxytryptamine)6 (5-HT₆) receptor antagonists, serotonin (5-HT) reuptake inhibitors and trophic factors.

The “other PDE9 inhibitors” and “other phosphodiesterase (PDE) inhibitors” refer to medicaments in research or on the market that have demonstrated activity on PDE9 or PDE.

The present invention further provides a pharmaceutical formulation comprising the aforementioned compound of formula (I) or (II) or the pharmaceutically acceptable salt or the isomer thereof.

In some embodiments of the present invention, the pharmaceutical formulation may comprise one or more pharmaceutical carriers.

The pharmaceutical carrier described herein may be one or more solid or liquid fillers or gel materials suitable for administration in human. The pharmaceutical carrier has sufficient purity and sufficiently low toxicity, and is compatible with the compound or the pharmaceutically acceptable salt or the isomer thereof provided herein without significantly decreasing its efficacy. For example, the pharmaceutical carrier may be a filler, a binder, a disintegrant, a lubricant, an aqueous solvent, a nonaqueous solvent, and the like.

The pharmaceutical formulation disclosed herein may be formulated into any pharmaceutically acceptable dosage form, and can be administered to a patient or a subject in need of such treatment in any suitable route of administration, such as oral, parenteral, rectal or pulmonary administration. For oral administration, it can be formulated into tablets, capsules, pills, granules, and the like. For parenteral administration, it can be formulated into injections, sterile powders for injection, and the like.

The present invention further provides use of the aforementioned compound of formula (I) or (II) or the pharmaceutically acceptable salt or the isomer thereof, the aforementioned pharmaceutical formulation or the aforementioned pharmaceutical composition in the manufacture of a medicament for treating or preventing a PDE9-mediated related disease; specifically, the PDE9-mediated related disease is cognitive impairment caused by central nervous system disorders; and more specifically, the cognitive impairment includes impairments of perception, concentration, memory and learning, including but not limited to senile dementia, schizophrenia, age-related memory loss, vascular dementia, craniocerebral trauma, stroke, post-stroke dementia, post-traumatic dementia, general concentration impairment, concentration impairments in children with learning and memory problems, Alzheimer's disease, Lewy body dementia, dementia with degeneration of the frontal lobes, dementia with corticobasal degeneration, amyotrophic lateral sclerosis, Huntington's disease, multiple sclerosis, thalamic degeneration, Creutzfeldt-Jakob dementia, HIV dementia, schizophrenia, Korsakoffs psychosis, and/or depression or bipolar disorder.

The present invention further provides use of the aforementioned compound of formula (I) or (II) or the pharmaceutically acceptable salt or the isomer thereof, the aforementioned pharmaceutical formulation or the aforementioned pharmaceutical composition in treating or preventing diseases.

The present invention further provides use of the aforementioned compound of formula (I) or (II) or the pharmaceutically acceptable salt or the isomer thereof, the aforementioned pharmaceutical formulation or the aforementioned pharmaceutical composition in treating or preventing the PDE9-mediated related disease; specifically, the PDE9-mediated related disease is cognitive impairment caused by central nervous system disorders; and more specifically, the cognitive impairment includes impairments of perception, concentration, memory and learning, including but not limited to senile dementia, schizophrenia, age-related memory loss, vascular dementia, craniocerebral trauma, stroke, post-stroke dementia, post-traumatic dementia, general concentration impairment, concentration impairments in children with learning and memory problems, Alzheimer's disease, Lewy body dementia, dementia with degeneration of the frontal lobes, dementia with corticobasal degeneration, amyotrophic lateral sclerosis, Huntington's disease, multiple sclerosis, thalamic degeneration, Creutzfeldt-Jakob dementia, HIV dementia, schizophrenia, Korsakoffs psychosis, and/or depression or bipolar disorder.

The present invention further provides a method for treating or preventing a disease, comprising administering to a patient in need thereof a therapeutically effective amount of the aforementioned compound of formula (I) or (II) or the pharmaceutically acceptable salt or the isomer thereof, the aforementioned pharmaceutical formulation or the aforementioned pharmaceutical composition, wherein the disease is the PDE9-mediated related disease; specifically, the PDE9-mediated related disease is cognitive impairment caused by central nervous system disorders; and more specifically, the cognitive impairment includes impairments of perception, concentration, memory and learning, including but not limited to senile dementia, schizophrenia, age-related memory loss, vascular dementia, craniocerebral trauma, stroke, post-stroke dementia, post-traumatic dementia, general concentration impairment, concentration impairments in children with learning and memory problems, Alzheimer's disease, Lewy body dementia, dementia with degeneration of the frontal lobes, dementia with corticobasal degeneration, amyotrophic lateral sclerosis, Huntington's disease, multiple sclerosis, thalamic degeneration, Creutzfeldt-Jakob dementia, HIV dementia, schizophrenia, Korsakoffs psychosis, and/or depression or bipolar disorder.

### DETAILED DESCRIPTION OF THE INVENTION

The “halogen” described herein refers to fluorine, chlorine, bromine, iodine, and the like, and preferably fluorine and chlorine.

The “halogenated” described herein means that any hydrogen atom in a substituent can be substituted with one or more identical or different halogen atoms. “Halogen” is defined as above.

The “C₁₋₆ alkyl” described herein refers to linear or branched alkyl derived by removing one hydrogen atom from a hydrocarbon moiety containing 1 to 6 carbon atoms, such as methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl and 1-methyl-2-methylpropyl. The “C₁₋₄ alkyl” refers to the aforementioned examples containing 1 to 4 carbon atoms.

The “C₂₋₈ alkenyl” described herein refers to linear, branched or cyclic alkenyl derived by removing one hydrogen atom from an alkene moiety containing 2 to 8 carbon atoms and a carbon-carbon double bond, such as vinyl, 1-propenyl, 2-propenyl, 1-butenyl, 2-butenyl, 1,3-butadienyl, 1-pentenyl, 2-pentenyl, 3-pentenyl, 1,3-pentadienyl, 1,4-pentadienyl, 1-hexenyl and 1,4-hexadienyl.

The “C₂₋₈ alkynyl” described herein refers to linear or branched alkynyl derived by removing one hydrogen atom from an alkyne moiety containing 2 to 8 carbon atoms and a carbon-carbon triple bond, such as ethynyl, propynyl, 2-butynyl, 2-pentynyl, 3-pentynyl, 4-methyl-2-pentynyl, 2-hexynyl and 3-hexynyl.

The “C₁₋₆ alkoxy” described herein refers to a group in which the “C₁₋₆ alkyl” defined above is linked to a parent molecule via an oxygen atom, i.e., a “C₁₋₆ alkyl-O—” group, such as methoxy, ethoxy, n-propoxy, isopropoxy, n-butoxy, tert-butoxy, n-pentyloxy, neopentyloxy and n-hexyloxy. The “C₁₋₄ alkoxy” refers to the aforementioned examples containing 1 to 4 carbon atoms, i.e., a “C₁₋₄ alkyl-O—” group.

The “C₁₋₆ alkylamino”, “(C₁₋₆ alkyl)₂amino”, “C₁₋₆ alkylcarbonylamino”, “C₁₋₆ alkylsulfonylamino”, “C₁₋₆ alkylaminocarbonyl”, “(C₁₋₆ alkyl)₂aminocarbonyl”, “C₁₋₆ alkoxycarbonyl”, “C₁₋₆ alkylsulfonyl”, “C₁₋₆ alkylthio” and “C₁₋₆ alkylcarbonyl” refer to C₁₋₆ alkyl-NH—, (C₁₋₆ alkyl)(C₁₋₆ alkyl)N—, C₁₋₆ alkyl-C(O)—NH—, C₁₋₆ alkyl-S(O)₂—NH—, C₁₋₆ alkyl-NH—C(O)—, (C₁₋₆ alkyl)(C₁₋₆ alkyl)N—C(O)—, C₁₋₆ alkyl-O—C(O)—, C₁₋₆ alkyl-S(O)₂—, C₁₋₆ alkyl-S— and C₁₋₆ alkyl-C(O)—, respectively, wherein the “C₁₋₆ alkyl” is defined as above, and is preferably “C₁₋₄ alkyl”.

The “polycyclic ring” described herein refers to a multi-ring system structure formed by two or more ring structures connected by an ortho-fused, spiro- or bridged linkage. The ortho-fused ring refers to a polycyclic structure formed by two or more ring structures sharing two adjacent ring atoms (i.e., sharing a bond) with each other. The bridged ring refers to a polycyclic structure formed by two or more ring structures sharing two non-adjacent ring atoms with each other. The spiro-ring refers to a polycyclic structure formed by two or more ring structures sharing a ring atom with each other.

Unless otherwise specified, the “3-12 membered cycloalkenyl” described herein includes all possibly formed monocyclic and polycyclic (including fused in the form of ortho-, spiro- or bridged) cases, such as 3-8 membered monocyclic cycloalkenyl, 7-11 membered spiro-cycloalkenyl, 7-11 membered ortho-fused cycloalkenyl and 6-11 membered bridged cycloalkenyl.

The cycloalkyl described herein includes all possibly formed monocyclic and polycyclic (including fused in the form of ortho-, spiro- or bridged) cases. For example, “3-12 membered cycloalkyl” can be a monocyclic, bicyclic or polycyclic cycloalkyl system (also referred to as a polycyclic ring system). Unless otherwise specified, the monocyclic ring system is a cyclic hydrocarbon group containing 3 to 8 carbon atoms. Examples of 3-8 membered cycloalkyl include, but are not limited to: cyclopropyl, cyclobutyl, cyclopentyl, cyclohexyl, cycloheptyl, cyclooctyl, and the like. Polycyclic cycloalkyl includes ortho-fused cycloalkyl, bridged cycloalkyl and spiro-cycloalkyl. Ortho-fused cycloalkyl may be 6-11 membered ortho-fused cycloalkyl or 7-10 membered ortho-fused cycloalkyl, and the representative examples thereof include, but are not limited to, bicyclo[3.1.1]heptane, bicyclo[2.2.1]heptane, bicyclo[2.2.2]octane, bicyclo[3.2.2]nonane, bicyclo[3.3.1]nonane and bicyclo[4.2.1]nonane. The spiro-cycloalkyl may be 7-12 membered spiro-cycloalkyl or 7-11 membered spiro-cycloalkyl, and the examples thereof include, but are not limited to:

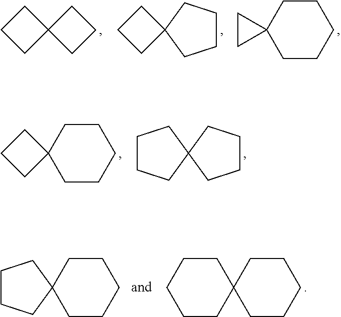

The bridged cycloalkyl may be 6-11 membered bridged cycloalkyl or 7-10 membered bridged cycloalkyl, and the examples thereof include, but are not limited to:

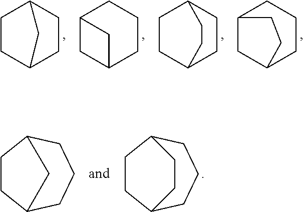

The “heterocyclyl” described herein refers to a 3-12 membered non-aromatic cyclic group in which at least one ring carbon atom is replaced with a heteroatom selected from O, S and N, and preferably 1 to 3 heteroatoms are present, wherein a carbon atom, a nitrogen atom and a sulfur atom may be oxidized.

“3-12 membered heterocyclyl” refers to a monocyclic heterocyclyl, bicyclic heterocyclyl, or polycyclic heterocyclyl system (also referred to as a fused ring system), including saturated and partially saturated heterocyclyl groups, but excluding aromatic rings. Unless otherwise specified, all possibly formed monocyclic, polycyclic (including fused in the form of ortho-, spiro- or bridged), saturated and partially saturated cases are included.

The monocyclic heterocyclyl may be 3-8 membered heterocyclyl, 3-8 membered saturated heterocyclyl, 3-6 membered heterocyclyl, 4-7 membered heterocyclyl, 5-7 membered heterocyclyl, 5-6 membered heterocyclyl, 5-6 membered oxygen-containing heterocyclyl, 3-8 membered nitrogen-containing heterocyclyl, 5-6 membered nitrogen-containing heterocyclyl, 5-6 membered saturated heterocyclyl, or the like. Examples of the “3-8 membered saturated heterocyclyl” include, but are not limited to, aziridinyl, oxiranyl, thiiranyl, azetidinyl, oxetanyl, thietanyl, tetrahydrofuranyl, pyrrolidinyl, tetrahydrothienyl, imidazolidinyl, pyrazolidinyl, 1,2-oxazolidinyl, 1,3-oxazolidinyl, 1,2-thiazolidinyl, 1,3-thiazolidinyl, tetrahydro-2H-pyranyl, tetrahydro-2H-thiapyranyl, piperidinyl, piperazinyl, morpholinyl, 1,4-dioxanyl and 1,4-oxathianyl. Examples of the “3-8 membered partially saturated heterocyclyl” include, but are not limited to, 4,5-dihydroisoxazolyl, 4,5-dihydrooxazolyl, 2,5-dihydrooxazolyl, 2,3-dihydrooxazolyl, 3,4-dihydro-2H-pyrrolyl, 2,3-dihydro-TH-pyrrolyl, 2,5-dihydro-TH-imidazolyl, 4,5-dihydro-TH-imidazolyl, 4,5-dihydro-1H-pyrazolyl, 4,5-dihydro-3H-pyrazolyl, 4,5-dihydrothiazolyl, 2,5-dihydrothiazolyl, 2H-pyranyl, 4H-pyranyl, 2H-thiapyranyl, 4H-thiapyranyl, 2,3,4,5-tetrahydropyridinyl, 1,2-isoxazinyl, 1,4-isoxazinyl, 6H-1,3-oxazinyl and the like. Polycyclic heterocyclyl includes ortho-fused heterocyclyl, spiro-heterocyclyl and bridged heterocyclyl, which may be saturated, partially saturated or unsaturated, but non-aromatic. Polycyclic heterocyclyl may be 5-6 membered monocyclic heterocyclyl ring which is fused to a benzene ring, 5-6 membered monocyclic cycloalkyl, 5-6 membered monocyclic cycloalkenyl, 5-6 membered monocyclic heterocyclyl or 5-6 membered monocyclic heteroaryl. The ortho-fused heterocyclyl may be 6-12 membered ortho-fused heterocyclyl, 7-10 membered ortho-fused heterocyclyl, 6-10 membered ortho-fused heterocyclyl or 6-12 membered saturated ortho-fused heterocyclyl, and representative examples include, but are not limited to: 3-azabicyclo[3.1.0]hexyl, 3,6-diazabicyclo[3.2.0]heptyl, 3,8-diazabicyclo [4.2.0]octyl, 3,7-diazabicyclo[4.2.0]octyl, octahydropyrrolo[3,4-c] pyrrolyl, octahydropyrrolo[3,4-b]pyrrolyl, octahydropyrrolo[3,4-b][1,4]oxazinyl, octahydro-1H-pyrrolo[3,4-c]pyridinyl, 2,3-dihydrobenzofuran-2-yl, 2,3-dihydrobenzofuran-3-yl, indolin-1-yl, indolin-2-yl, indolin-3-yl, 2,3-dihydrobenzothiophen-2-yl, octahydro-TH-indolyl and octahydrobenzofuranyl. The spiro-heterocyclyl may be 6-12 membered spiro-heterocyclyl, 7-11 membered spiro-heterocyclyl or 6-12 membered saturated spiro-heterocyclyl, and examples thereof include, but are not limited to:

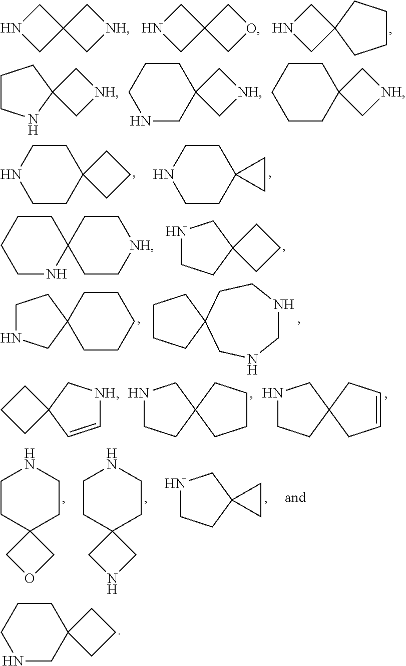

The bridged heterocyclyl may be 6-12 membered bridged heterocyclyl, 7-11 membered bridged heterocyclyl or 6-12 membered saturated bridged heterocyclyl, and examples thereof include, but are not limited to:

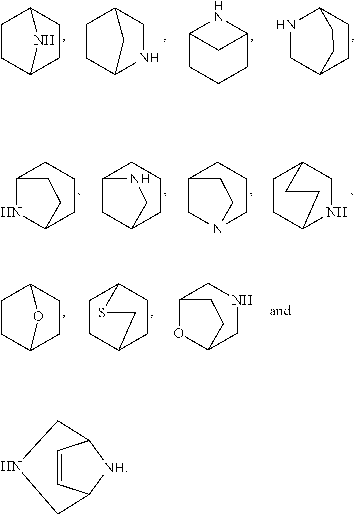

The “aryl” described herein refers to a cyclic aromatic group containing 6 to 14 carbon atoms, including phenyl, naphthalene, phenanthrene, and the like.

The heteroaryl described herein includes all possibly formed monocyclic, polycyclic, fully aromatic and partially aromatic cases. For example, “5-10 membered heteroaryl” refers to an aromatic cyclic group in which at least one ring carbon atom is substituted with a heteroatom selected from O, S and N, and preferably 1 to 3 heteroatoms are present. Moreover, the case where carbon atoms or sulfur atoms are oxidized is included. For example, carbon atoms are replaced with C(O) and sulfur atoms are substituted by S(O) or S(O)₂. Unless otherwise specified, heteroaryl includes monocyclic heteroaryl and polycyclic heteroaryl. Monocyclic heteroaryl may be 5-7 membered heteroaryl or 5-6 membered heteroaryl, and examples thereof include, but are not limited to, furanyl, imidazolyl, isoxazolyl, thiazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thienyl, triazolyl and triazinyl. In certain examples, polycyclic heteroaryl refers to a group in which a monocyclic heteroaromatic ring is fused to phenyl, cycloalkenyl, heteroaryl, cycloalkyl or heterocyclyl. Polycyclic heteroaryl may be 8-12 membered ortho-fused heteroaryl or 9-10 membered ortho-fused heteroaryl, and examples include, but are not limited to, benzimidazolyl, benzofuranyl, benzothienyl, benzoxadiazolyl, benzothiadiazolyl, benzothiazolyl, cinnolinyl, 5,6-dihydroquinolin-2-yl, 5,6-dihydroisoquinolin-1-yl, furopyridinyl, indazolyl, indolyl, isoindolyl, isoquinolinyl, naphthyridinyl, purinyl, quinolinyl, 5,6,7,8-tetrahydroquinolin-2-yl, 5,6,7,8-tetrahydroquinolinyl, 5,6,7,8-tetrahydroquinolin-4-yl, 5,6,7,8-tetrahydroisoquinolin-1-yl, thienopyridinyl, 4,5,6,7-tetrahydro[c][1,2,5]oxadiazolyl and 6,7-dihydro[c][1,2,5]oxadiazol-4(5H)keto.

The “pharmaceutically acceptable salt” described herein refers to a pharmaceutically acceptable addition salt of acid and base or a solvate thereof. Such pharmaceutically acceptable salts include salts of the following acids: hydrochloric acid, phosphoric acid, hydrobromic acid, sulfuric acid, sulfurous acid, formic acid, toluenesulfonic acid, methanesulfonic acid, nitric acid, benzoic acid, citric acid, tartaric acid, maleic acid, hydroiodic acid, alkanoic acid (such as acetic acid, HOOC—(CH₂)n-COOH (wherein n is 0-4)), and the like. The following salts of bases are also included: sodium salt, potassium salt, calcium salt, ammonium salt, and the like. Those skilled in the art know a variety of pharmaceutically acceptable non-toxic addition salts.

The “isomer” described herein refers to a stereoisomer and a tautomer.

The stereoisomer refers to an enantiomer in the case that an asymmetric carbon atom exists in a compound, and a cis-trans isomer in the case that a carbon-carbon double bond or a cyclic structure exists in a compound. All enantiomers, diastereomers, racemic isomers, cis-trans isomers, geometric isomers, epimers and mixtures thereof of the compound of formula (I) are included in the scope of the present invention.

The “tautomer” refers to a functional group isomer that is produced due to the rapid shifting of a certain atom between two positions in a molecule, and the tautomer is a special functional group isomer. Examples include tautomerization of a carbonyl compound containing α-H, specifically as follows:

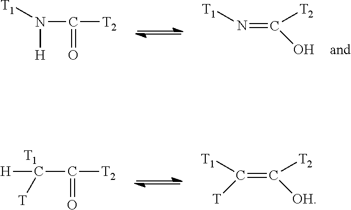

The tautomerization may also be, for example, other prototropic tautomerizations, specifically such as phenol-keto tautomerization, nitroso-oximino tautomerization and imine-enamine tautomerization.

T, T1 and T2 are each independently any group that accords with the bonding rule of a compound.

The compound disclosed herein contains a lactam structure and involves the following tautomerization:

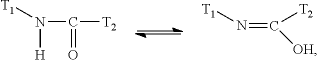

and therefore when referring to the compound disclosed herein, it means that the tautomers of the compound are also referred to.

The “therapeutically effective amount” disclosed herein refers to an amount of the aforementioned compound or the pharmaceutically acceptable salts or isomers thereof, the composition or the pharmaceutical formulation thereof, that, when administered to a patient, is at least capable of alleviating symptoms of the patient's condition. An actual amount comprising the “therapeutically effective amount” will vary depending on a variety of circumstances, including, but not limited to, the particular condition being treated, the severity of the condition, the physique and health of the patient, and the route of administration. The appropriate amount can be readily determined by skilled medical practitioners using methods known in the medical field.

Compound Preparation

The compound disclosed herein can be prepared by a variety of methods including standard chemical methods. Unless otherwise stated, any variable defined above will continue to have the meaning defined above. Exemplary general synthesis methods are elaborated in the following schemes, and can be easily modified to prepare other compounds disclosed herein. The specific compounds disclosed herein were prepared in examples.

In some embodiments, the compound of formula (I) can be prepared by metal-catalyzed coupling, aromatic nucleophilic substitution or other reactions of a compound of formula (I-d) and a compound of formula (I-e), which is shown as follows:

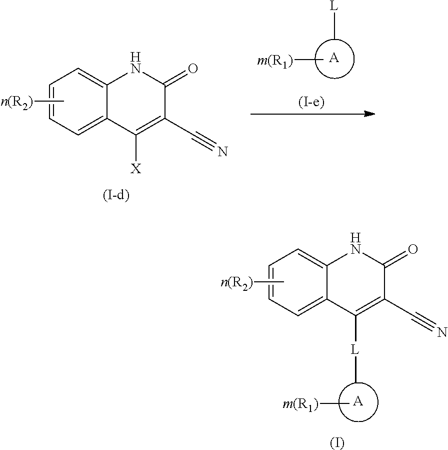

In some embodiments, the compound of formula (I-d) can be prepared by the reaction of a compound of formula (I-c) with a halogenating agent, or substituted or unsubstituted sulfonyl chloride or sulfonic anhydride, which is shown as follows:

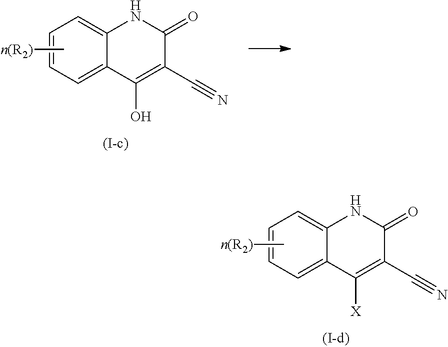

In some embodiments, the compound of formula (I-c) can be prepared by cyclization of a compound of formula (I-b) in the presence of a suitable base, which is shown as follows:

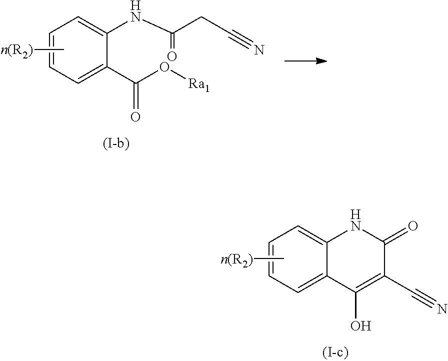

In some embodiments, the compound of formula (I-b) can be prepared by reaction of a compound of formula (I-a) with cyanoacetic acid in the presence of a suitable peptide coupling agent, which is shown as follows:

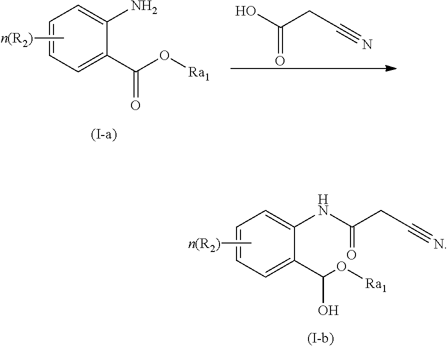

In the above embodiments, R₁, R₂, A, L, m and n are defined as above, Rai is selected from hydrogen and C₁₋₆ alkyl, and X is selected from halogen, substituted or unsubstituted benzene sulfonate, C₁₋₆ alkyl sulfonate, triflate, and the like.

In some embodiments, the compound of formula (I) can be prepared by metal-catalyzed coupling reaction of a compound of formula (I-j) with a suitable reagent comprising an R₂ group, or by metal-catalyzed coupling reaction of a compound of formula (I-j) with a suitable reagent followed by one or more conventional chemical transformations (such as oxidation, reduction, addition, substitution, hydrogenation, chlorination and amination), which is shown as follows:

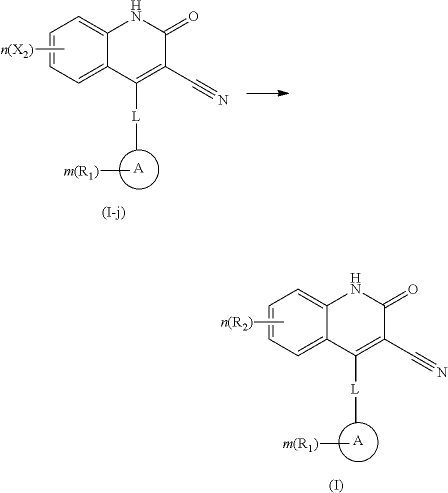

In some embodiments, the compound of (I-j) can be prepared by aromatic nucleophilic substitution or other reactions of a compound of formula (I-i) with the compound of formula (I-e), which is shown as follows:

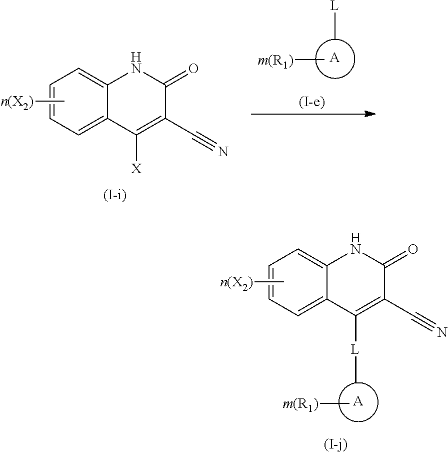

In some embodiments, the compound of formula (I-i) can be prepared by the reaction of a compound of formula (I-h) with a halogenating agent, or substituted or unsubstituted sulfonyl chloride or sulfonic anhydride, which is shown as follows:

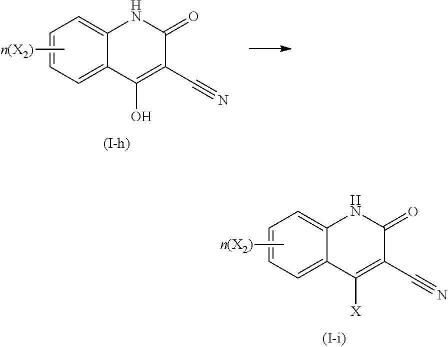

In some embodiments, the compound of formula (I-h) can be prepared by cyclization of a compound of formula (I-g) in the presence of a suitable base, which is shown as follows:

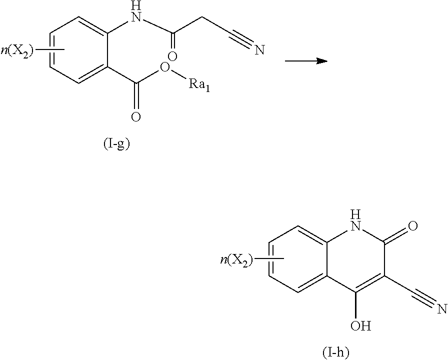

In some embodiments, the compound of formula (I-g) can be prepared by reaction of a compound of formula (I-f) with cyanoacetic acid in the presence of a suitable peptide coupling agent, which is shown as follows:

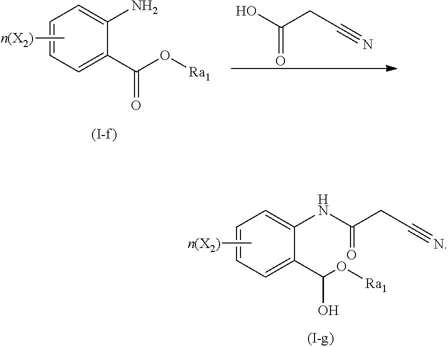

In the above embodiments, R₁, R₂, A, L, m, n, Ra₁, and X are defined as above, and X₂ is selected from bromine and iodine.

The halogenating reagent refers to reagents used in the halogenation reaction, including but not limited to N-bromosuccinimide, N-chlorosuccinimide, N-iodosuccinimide, dibromohydantoin, phosphorus tribromide, phosphine trichloride, thionyl chloride, phosphorus oxychloride, phosphorus pentachloride, or phosphorus oxybromide.

The suitable base includes organic and inorganic bases. The organic bases include, but are not limited to, sodium tert-butoxide, potassium tert-butoxide, sodium ethoxide, sodium methoxide, LiHMDS, N,N-diisopropylethylamine, triethylamine, lithium diisopropylamide, and the like. The inorganic bases include, but are not limited to, sodium hydride, sodium carbonate, potassium carbonate, cesium carbonate, potassium phosphate, potassium hydroxide, sodium hydroxide, magnesium hydroxide, calcium hydroxide, and the like.

The substituted or unsubstituted sulfonyl chloride refers to Ra₂—SO₂Cl, and

the substituted or unsubstituted sulfonic anhydride refers to (Ra₂—SO₂)₂—O,

wherein Ra₂ is selected from C₁₋₆ alkyl, halogenated C₁₋₆ alkyl, substituted or unsubstituted aryl, and the like.

The peptide coupling agent refers to agents capable of activating carboxylic acid to form amides with amines, and includes, but is not limited to, 1-(3-dimethylaminopropyl)-3-ethylcarbodiimide hydrochloride, 2-(7-azabenzotriazol)-N,N, N′,N′-tetramethyluronium hexafluorophosphate, O-tetramethyluronium hexafluorophosphate, N,N′-carbonyldiimidazole, benzotriazol-1-yloxytris(dimethylamino)phosphonium hexafluorophosphate, propylphosphoric anhydride, carbodiimide, and the like.

### EXAMPLES

Abbreviations used herein:

“DCM” refers to dichloromethane, “DMF” refers to N,N-dimethylformamide, “DIPEA” refers to N,N-diisopropylethylamine, “PE” refers to petroleum ether, “EA” refers to ethyl acetate, “EDCI” refers to carbodiimide, “HATU” refers to 2-(7-azabenzotriazol)-N,N,N′,N′-tetramethyluronium hexafluorophosphate, and “THF” refers to tetrahydrofuran.

### Example 1: Synthesis of 6-bromo-4-(4-methoxy-4-methylpiperidin-1-yl)-2-oxo-1,2-dihydroquinoline-3-carbonitrile (Compound 180)

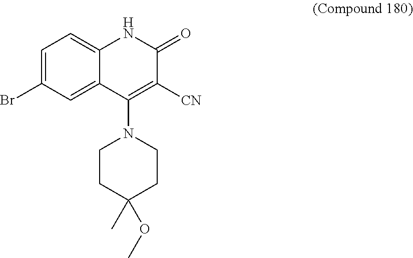 [SMILES: COC1(C)CCN(c2c(C#N)c(=O)[nH]c3ccc(Br)cc23)CC1]

### Step 1: Synthesis of ethyl 2-amino-5-bromobenzoate

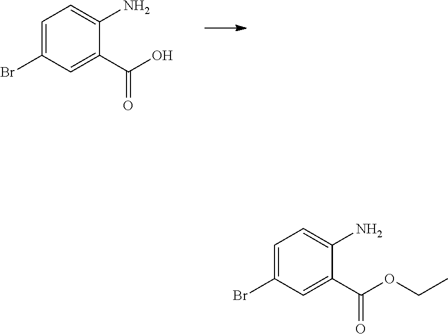

Intermediate 2-amino-5-bromobenzoic acid (50 g, 0.231 mol, 1.0 eq) and concentrated sulfuric acid (20 mL) were dissolved in absolute ethanol (500 mL), and the reaction mixture was reacted at 80° C. for 96 h. After the reaction completion as detected by LC-MS, the reaction mixture was concentrated under reduced pressure, added with ethyl acetate (200 mL), and adjusted to pH=10 by adding saturated aqueous potassium carbonate solution at 0° C., followed by liquid separation. The aqueous phase was extracted with ethyl acetate (200 mL×2), and the organic phases were combined, dried over anhydrous sodium sulfate, filtered, and then concentrated under reduced pressure. The crude product was slurried with PE:EA (100:1, 200 mL) and filtered under vacuum, and the filter cake was dried to give the product (37 g, yield: 65%).

### Step 2: Synthesis of ethyl 5-bromo-2-(2-cyanoacetamido)benzoate

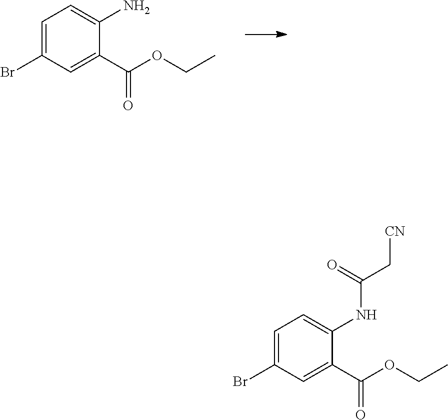 [SMILES: CCOC(=O)c1cc(Br)ccc1N ; CCOC(=O)c1cc(Br)ccc1NC(=O)CC#N]

Intermediate ethyl 2-amino-5-bromobenzoate (37 g, 0.151 mol, 1.0 eq) and cyanoacetic acid (15.5 g, 0.182 mol, 1.2 eq) were dissolved in DCM (400 mL), and the reaction mixture was then cooled to 0° C., added with EDCI (43.4 g, 0.227 mol, 1.5 eq) in batches, and reacted for 50 min. After the reaction completion as detected by TLC, the reaction mixture was added with water (300 mL), stirred for 30 min and filtered under vacuum. The filter cake was collected and rinsed with water (300 mL), and the filtrate was subjected to liquid separation. The aqueous phase was extracted with ethyl acetate (200 mL×2), and the organic phases were combined, dried over anhydrous sodium sulfate, filtered and concentrated under reduced pressure. The crude product was slurried with PE/EA (20/1, 50 mL) and filtered under vacuum. The filter cakes in two batches were dried to give product (45.2 g, yield: 96%).

### Step 3: Synthesis of 6-bromo-4-hydroxy-2-oxo-1,2-dihydroquinoline-3-carbonitrile

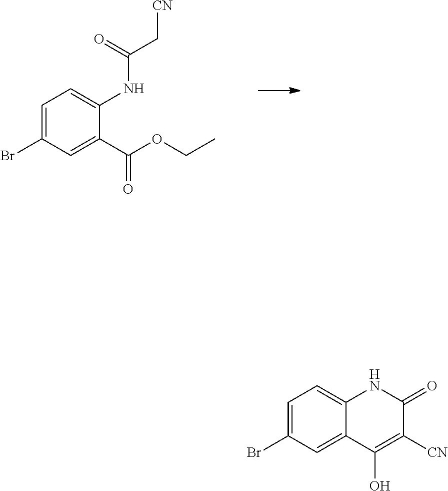 [SMILES: CCOC(=O)c1cc(Br)ccc1NC(=O)CC#N ; N#Cc1c(O)c2cc(Br)ccc2[nH]c1=O]

Intermediate ethyl 5-bromo-2-(2-cyanoacetamido)benzoate (45.2 g, 0.145 mol, 1.0 eq) was dissolved in ethanol (600 mL), and sodium ethoxide (29.58 mg, 0.435 mol, 1.5 eq) was added under an ice bath, and then the reaction mixture was reacted for 30 min. After the reaction completion as detected by TLC, the reaction mixture was concentrated under reduced pressure, added with water (500 mL), and adjusted to pH=2 with concentrated hydrochloric acid, and white solids were precipitated. The reaction mixture was then filtered, and the filter cake was dried to give the product (38.4 g, yield: 100%).

### Step 4: Synthesis of 6-bromo-2,4-dichloroquinoline-3-carbonitrile

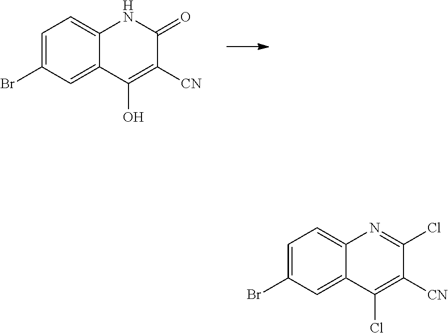 [SMILES: N#Cc1c(O)c2cc(Br)ccc2[nH]c1=O ; N#Cc1c(Cl)nc2ccc(Br)cc2c1Cl]

Intermediate 6-bromo-4-hydroxy-2-oxo-1,2-dihydroquinoline-3-carbonitrile (38.4 g, 0.145 mol, 1.0 eq) was dissolved in acetonitrile (200 mL), and phosphorus oxychloride (77.8 g, 0.507 mol, 3.5 eq) was added, and the reaction mixture was reacted at 90° C. for 3 h. After the reaction completion as detected by LC-MS, the reaction mixture was concentrated under reduced pressure to give a crude product, which was used in the next step according to a theoretical amount.

### Step 5: Synthesis of 6-bromo-4-chloro-2-oxo-1,2-dihydroquinoline-3-carbonitrile

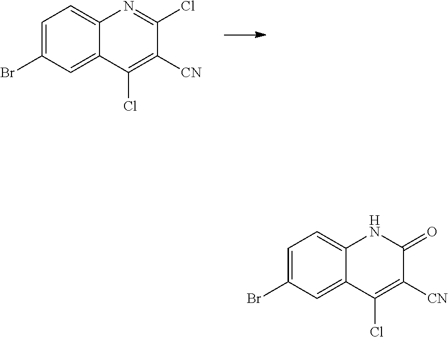 [SMILES: N#Cc1c(Cl)nc2ccc(Br)cc2c1Cl ; N#Cc1c(Cl)c2cc(Br)ccc2[nH]c1=O]

Intermediate 6-bromo-2,4-dichloroquinoline-3-carbonitrile (43.7 g, 0.145 mol, 1.0 eq) was dissolved in a mixed solvent of trifluoroacetic acid (300 mL) and water (80 mL), and then the reaction mixture was heated to 90° C. and reacted for 2.5 h. After the reaction completion as detected by TLC, the reaction mixture was added dropwise into ice water (1 L), and the resulting reaction mixture was stirred for 20 min and filtered under vacuum. The filter cake was dried to give the product (37.5 g, yield: 91%).

### Step 6: Synthesis of 6-bromo-4-(4-methoxy-4-methylpiperidin-1-yl)-2-oxo-1,2-dihydroquinoline-3-carbonitrile

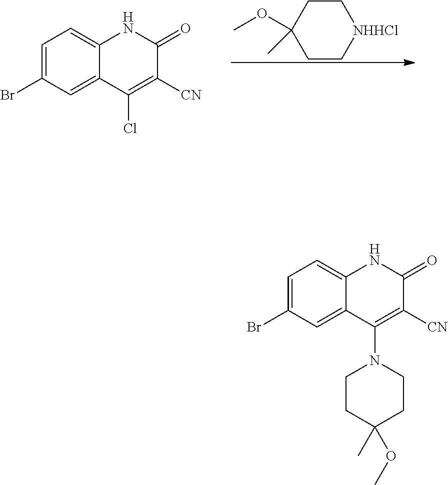

Intermediate 6-bromo-4-chloro-2-oxo-1,2-dihydroquinoline-3-carbonitrile (30 g, 0.106 mol, 1.0 eq), 4-methoxy-4-methylpiperidine hydrochloride (19.28 g, 0.116 mol, 1.1 eq) and DIPEA (41.12 g, 0.318 mol, 3.0 eq) were dissolved in DMF (150 mL), and the reaction mixture was reacted at 80° C. for 1 h. After the reaction completion as detected by LC-MS, the reaction mixture was added dropwise into ice water (750 mL), and the resulting reaction mixture was stirred for 30 min and filtered under vacuum. The filter cake was dried to give the product (30 g, yield: 75%).

¹HNMR (400 MHz, DMSO-d₆) δ (ppm): 11.91 (s, 1H), 7.79-7.80 (s, 1H), 7.73-7.76 (m, 1H), 7.23-7.25 (s, 1H), 3.55-3.58 (d, 2H), 3.50-3.52 (m, 2H), 3.18 (s, 3H), 1.88-1.91 (d, 2H), 1.72-1.79 (m, 2H), 1.22 (s, 3H).

Molecular formula: C₁₇H₁₈BrN₃O₂; Molecular weight: 376.25; LC-MS (Pos, m/z)=376.06[M+H]⁺.

### Example 2: Synthesis of 3-cyano-N-methyl-2-oxo-4-(6-azaspiro[2.5]octan-6-yl)-1,2-dihydroquinoline-6-carboxamide (Compound 96)

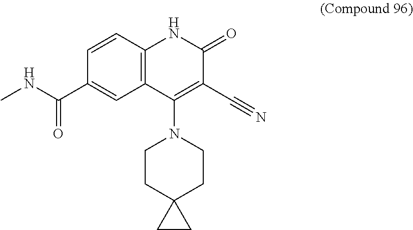 [SMILES: CNC(=O)c1ccc2[nH]c(=O)c(C#N)c(N3CCC4(CC3)CC4)c2c1]

### Step 1: Synthesis of 6-bromo-2-oxo-4-(6-azaspiro[2.5]octan-6-yl)-1,2-dihydroquinoline-3-carbonitrile

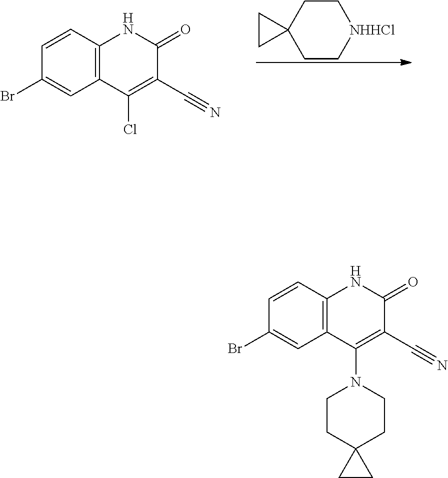

The product 6-bromo-4-chloro-2-oxo-1,2-dihydroquinoline-3-carbonitrile (7.0 g, 24.69 mmol, 1.0 eq) obtained in step 5 of Example 1 was dissolved in N,N-dimethylformamide (35 mL), and DIPEA (12.7 g, 98.76 mmol, 4.0 eq) and 6-azaspiro[2.5]octane hydrochloride (5.1 g, 34.56 mmol, 1.4 eq) were added. After the addition, the reaction mixture was heated to 80° C. and reacted for 1 h. After starting material disappearance as detected by LC-MS, the reaction mixture was cooled to room temperature and then poured into ice water (175 mL), and solids were precipitated. The reaction mixture was then filtered under vacuum, and the filter cake was washed with petroleum ether and dried to give the product (7.2 g, yield: 81.8%).

### Step 2: Synthesis of 2-oxo-4-(6-azaspiro[2.5]octan-6-yl)-6-vinyl-1,2-dihydroquinoline-3-carbonitrile

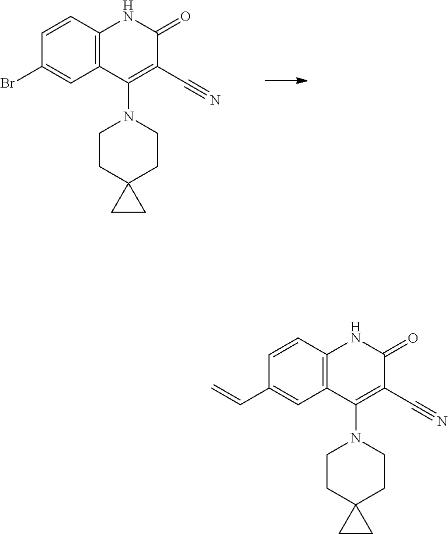 [SMILES: N#Cc1c(N2CCC3(CC2)CC3)c2cc(Br)ccc2[nH]c1=O ; C=Cc1ccc2[nH]c(=O)c(C#N)c(N3CCC4(CC3)CC4)c2c1]

Intermediate 6-bromo-2-oxo-4-(6-azaspiro[2.5]octan-6-yl)-1,2-dihydroquinoline-3-carbonitrile (7.2 g, 20 mmol, 1.0 eq), potassium vinylfluoroborate (4.0 g, 30 mmol, 1.5 eq) and cesium carbonate (19.5 g, 60 mmol, 3.0 eq) were dissolved in a mixed solvent of 1,4-dioxane (200 mL) and water (40 mL). After purge with nitrogen for three times, [1,1′-bis(diphenylphosphino)ferrocene]palladium dichloride (1.5 g, 2 mmol, 0.1 eq) was added. After purge with nitrogen for three times again, the reaction mixture was heated to 100° C. and reacted for 18 h. After starting material disappearance as detected by LC-MS, the reaction mixture was cooled to 60° C. and filtered. The filter cake was rinsed with ethyl acetate, and liquid separation was performed. The aqueous phase was extracted with ethyl acetate (50 mL×2), and the organic phases were combined, dried over anhydrous magnesium sulfate and filtered. The filtrate was concentrated under reduced pressure to give a crude product, which was slurried with methyl tert-butyl ether and filtered. The filter cake was dried to give the product (5.8 g, yield: 95%).

### Step 3: Synthesis of 6-formyl-2-oxo-4-(6-azaspiro[2.5]octan-6-yl)-1,2-dihydroquinoline-3-carbonitrile

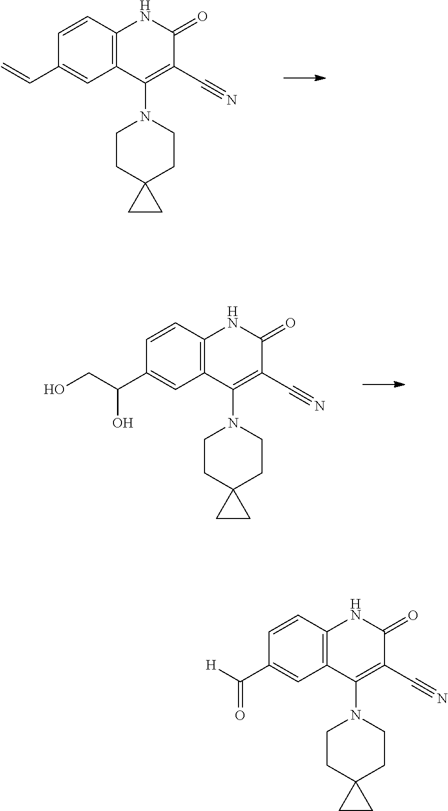 [SMILES: C=Cc1ccc2[nH]c(=O)c(C#N)c(N3CCC4(CC3)CC4)c2c1 ; N#Cc1c(N2CCC3(CC2)CC3)c2cc(C(O)CO)ccc2[nH]c1=O ; N#Cc1c(N2CCC3(CC2)CC3)c2cc(C=O)ccc2[nH]c1=O]

Intermediate 2-oxo-4-(6-azaspiro[2.5]octan-6-yl)-6-vinyl-1,2-dihydroquinoline-3-carbonitrile (2.8 g, 9.17 mmol, 1.0 eq) was dissolved in a mixed solvent of tert-butanol (60 mL) and water (60 mL), and methanesulfonamide (872.1 mg, 9.17 mmol, 1.0 eq) and AD-mix-β (33.6 g) were added, and the reaction mixture was stirred at room temperature under nitrogen atmosphere for 68 h. After starting material disappearance as detected by TLC, sodium periodate (3.9 g, 18.34 mmol, 2.0 eq), tetrahydrofuran (10 mL) and water (10 mL) were added. After the addition, the resulting reaction mixture was stirred at room temperature for 7 h. After the presence of remaining starting material as detected by LC-MS, sodium periodate (3.9 g, 18.34 mmol, 2.0 eq), tetrahydrofuran (10 mL) and water (10 mL) were supplemented. After the addition, the reaction mixture was stirred at room temperature for 16 h. After starting material disappearance as detected by LC-MS, the reaction mixture was filtered. The filter cake was slurried with dichloromethane (100 mL), and liquid separation was performed. The organic phase was dried over anhydrous magnesium sulfate and filtered, and the filter cake was rinsed with dichloromethane. The filtrate was concentrated under reduced pressure to give a crude product, which was purified by silica gel column chromatography (MeOH:DCM=1:100) to give the product (754 mg, yield: 26.9%).

### Step 4: Synthesis of 3-cyano-2-oxo-4-(6-azaspiro[2.5]octan-6-yl)-1,2-dihydroquinoline-6-carboxylic acid

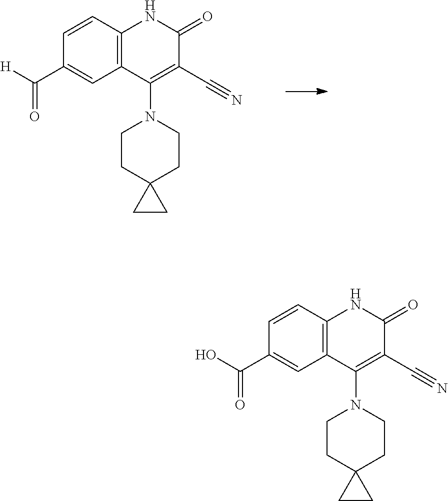 [SMILES: N#Cc1c(N2CCC3(CC2)CC3)c2cc(C=O)ccc2[nH]c1=O ; N#Cc1c(N2CCC3(CC2)CC3)c2cc(C(=O)O)ccc2[nH]c1=O]

Intermediate 6-formyl-2-oxo-4-(6-azaspiro[2.5]octan-6-yl)-1,2-dihydroquinoline-3-carbonitrile (754 mg, 2.45 mmol, 1.0 eq) was dissolved in formic acid (5 mL), and the reaction mixture was cooled to −5° C.-0° C. and added dropwise with 30% hydrogen peroxide (1.4 g, 12.26 mmol, 5.0 eq). After the addition, the reaction mixture was reacted for 16 h with the temperature maintained at −5° C.-0° C. Then the reaction mixture was added with water (50 mL) and extracted with dichloromethane (50 mL×3). The organic phases were combined, dried over anhydrous magnesium sulfate and filtered, and the filtrate was concentrated under reduced pressure to give a crude product, which was purified by silica gel column chromatography (MeOH:DCM=1:40). The resulting product was dissolved in water (20 mL), and the mixture was adjusted to pH=6 with 2 mol/L hydrochloric acid and extracted with ethyl acetate (10 mL×3), and the aqueous phase was lyophilized to give the product (183 mg, yield: 23%).

### Step 5: Synthesis of 3-cyano-N-methyl-2-oxo-4-(6-azaspiro[2.5]octan-6-yl)-1,2-dihydroquinoline-6-carboxamide

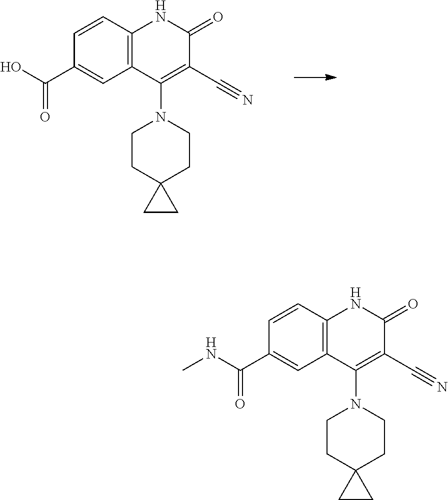 [SMILES: N#Cc1c(N2CCC3(CC2)CC3)c2cc(C(=O)O)ccc2[nH]c1=O ; CNC(=O)c1ccc2[nH]c(=O)c(C#N)c(N3CCC4(CC3)CC4)c2c1]

Intermediate 3-cyano-2-oxo-4-(6-azaspiro[2.5]octan-6-yl)-1,2-dihydroquinoline-6-carboxylic acid (170 mg, 0.53 mmol, 1.0 eq) was dissolved in N,N-dimethylacetamide (2 mL), and DIPEA (339 mg, 2.63 mmol, 5.0 eq) was added. Under nitrogen atmosphere, the reaction mixture was cooled to 0° C. in ice water, added with HATU (400 mg, 1.05 mmol, 2.0 eq), and reacted at room temperature for 1 h. Then the reaction mixture was added with methylamine hydrochloride (70.8 mg, 1.05 mmol, 2.0 eq) and reacted at room temperature for 1 h. After starting material disappearance as detected by LC-MS, the reaction mixture was added with water (50 mL) and extracted with ethyl acetate (50 mL×3). The organic phases were combined, washed with water (50 mL×2), dried over anhydrous magnesium sulfate and filtered, and the filtrate was concentrated under reduced pressure to give a crude product, which was purified by preparative thin-layer chromatography to give the product (27 mg, yield: 15.2%).

¹HNMR (400 MHz, DMSO-d₆) δ (ppm): 11.98 (s, 1H), 8.54-8.53 (m, 1H), 8.30-8.29 (s, 1H), 8.02-7.99 (d, 1H), 7.31-7.29 (d, 1H), 3.65-3.62 (m, 4H), 2.81-2.80 (s, 3H), 1.64-1.63 (m, 4H), 0.45 (m, 4H).

Molecular formula: C₁₉H₂₀N₄O₂; Molecular weight: 336.40; LC-MS (Pos, m/z)=337.4[M+H]⁺.

### Example 3: Synthesis of 6-ethyl-4-(4-methoxy-4-methylpiperidin-1-yl)-2-oxo-1,2-dihydroquinoline-3-carbonitrile (Compound 107)

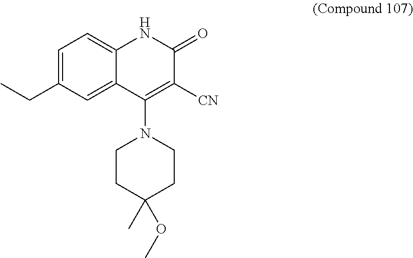 [SMILES: CCc1ccc2[nH]c(=O)c(C#N)c(N3CCC(C)(OC)CC3)c2c1]

### Step 1: Synthesis of 4-(4-methoxy-4-methylpiperidin-1-yl)-2-oxo-6-vinyl-1,2-dihydroquinoline-3-carbonitrile

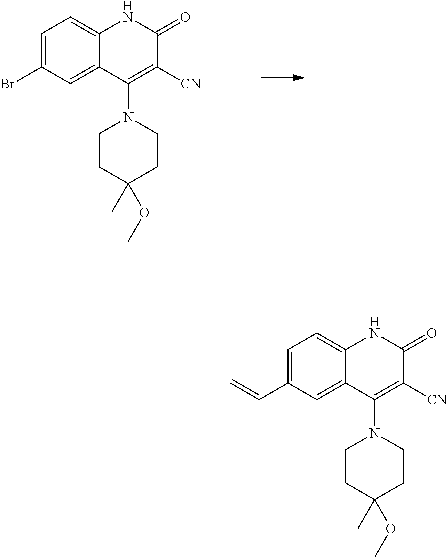 [SMILES: COC1(C)CCN(c2c(C#N)c(=O)[nH]c3ccc(Br)cc23)CC1 ; C=Cc1ccc2[nH]c(=O)c(C#N)c(N3CCC(C)(OC)CC3)c2c1]

The product 6-bromo-4-(4-methoxy-4-methylpiperidin-1-yl)-2-oxo-1,2-dihydroquinoline-3-carbonitrile (20 g, 0.053 mol, 1.0 eq) obtained in step 6 of Example 1 was dissolved in a mixed solvent of 1,4-dioxane (300 mL) and water (80 mL), and potassium vinyltrifluoroborate (10.68 g, 0.08 mmol, 1.5 eq) and cesium carbonate (51.8 g, 0.159 mol, 3.0 eq) were added. After purge with nitrogen, [1,1′-bis (diphenylphosphino)ferrocene]palladium dichloride (3.886 mg, 5.3 mmol, 0.1 eq) was added. Then the reaction mixture was reacted at 100° C. for 3 h under nitrogen atmosphere. After the reaction completion as detected by LC-MS, liquid separation was performed. The aqueous phase was extracted with dichloromethane (200 mL×3), and the organic phases were combined, dried over anhydrous sodium sulfate, filtered, and concentrated under reduced pressure to give a crude product, which was purified by silica gel column chromatography (DCM:MeOH=60:1) to give the product (14.3 g, yield: 83%).

### Step 2: Synthesis of 6-ethyl-4-(4-methoxy-4-methylpiperidin-1-yl)-2-oxo-1,2-dihydroquinoline-3-carbonitrile

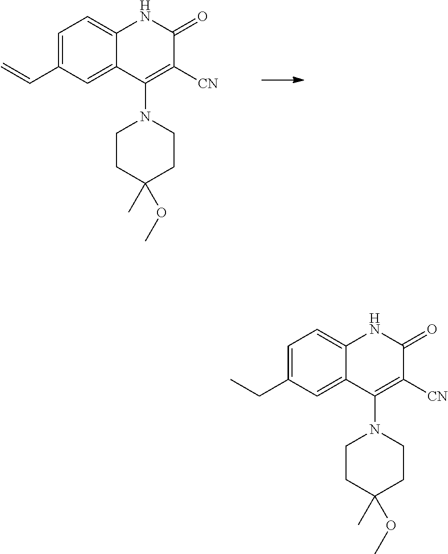 [SMILES: C=Cc1ccc2[nH]c(=O)c(C#N)c(N3CCC(C)(OC)CC3)c2c1 ; CCc1ccc2[nH]c(=O)c(C#N)c(N3CCC(C)(OC)CC3)c2c1]

Intermediate 4-(4-methoxy-4-methylpiperidin-1-yl)-2-oxo-6-vinyl-1,2-dihydroquinoline-3-carbonitrile (300 mg, 0.92 mmol, 1.0 eq) was dissolved in methanol (5 mL), and Pd/C (30 mg) was added. After purge with hydrogen for three times, the reaction mixture was reacted for 2 h under hydrogen atmosphere. After the reaction completion as detected by LC-MS, the reaction mixture was filtered under vacuum, and the filtrate was concentrated under reduced pressure to give the product (126 mg, yield: 42%).

¹HNMR (400 MHz, DMSO-d₆) δ (ppm): 11.71 (s, 1H), 7.50 (s, 1H), 7.45-7.47 (m, 1H), 7.21-7.23 (d, 1H), 3.5-3.61 (m, 4H), 3.18 (s, 3H), 2.64-2.70 (m, 2H), 1.88-1.92 (d, 2H), 1.74-1.81 (m, 2H), 1.22 (s, 6H).

Molecular formula: C₁₉H₂₃N₃O₂; Molecular weight: 325.41; LC-MS (Pos, m/z)=326.18[M+H]⁺.

### Example 4: Synthesis of 4-(4-methoxy-4-methylpiperidin-1-yl)-6-methyl-2-oxo-1,2-dihydroquinoline-3-carbonitrile (Compound 158)

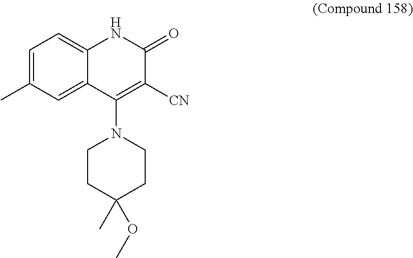 [SMILES: COC1(C)CCN(c2c(C#N)c(=O)[nH]c3ccc(C)cc23)CC1]

### Step 1: Synthesis of 4-(4-methoxy-4-methylpiperidin-1-yl)-6-methyl-2-oxo-1,2-dihydroquinoline-3-carbonitrile

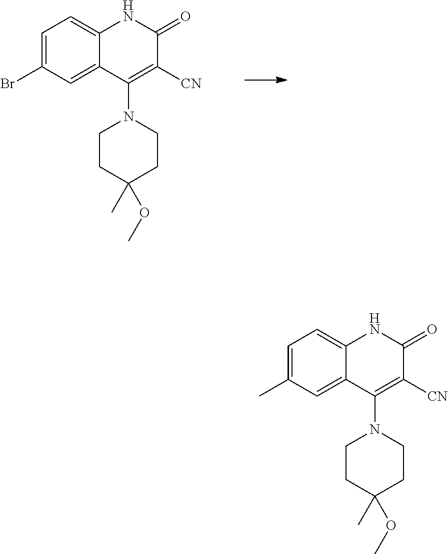 [SMILES: COC1(C)CCN(c2c(C#N)c(=O)[nH]c3ccc(Br)cc23)CC1 ; COC1(C)CCN(c2c(C#N)c(=O)[nH]c3ccc(C)cc23)CC1]

The product 6-bromo-4-(4-methoxy-4-methylpiperidin-1-yl)-2-oxo-1,2-dihydroquinoline-3-carbonitrile (500 mg, 1.33 mmol, 1.0 eq) obtained in step 6 of Example 1 was dissolved in a mixed solvent of 1,4-dioxane (5 mL) and water (1 mL), and cesium carbonate (1.3 g, 3.98 mol, 3.0 eq) and [1,1′-bis(diphenylphosphino)ferrocene]palladium dichloride (97 mg, 0.133 mmol, 0.1 eq) were added. After purge with nitrogen, trimethylboroxine (50% THF solution, 1.33 g, 5.31 mmol, 4.0 eq) was added. Then the reaction mixture was reacted at 100° C. for 12 h under nitrogen atmosphere. After the reaction completion as detected by LC-MS, liquid separation was performed. The aqueous phase was extracted with ethyl acetate (200 mL×3), and the organic phases were combined, dried over anhydrous sodium sulfate and filtered. The filtrate was concentrated under reduced pressure to give a crude product, which was purified by preparative HPLC (0.1% ammonia water:methanol=1:4) to give the product (66 mg, yield: 15%).

¹HNMR (400 MHz, DMSO-d₆) δ (ppm): 7.40-7.50 (d, 2H), 7.19-7.21 (d, 1H), 3.53-3.57 (m, 4H), 3.18 (s, 3H), 2.36 (s, 3H), 1.78-1.91 (m, 4H), 1.22 (s, 3H).

Molecular formula: C₁₈H₂₁N₃O₂; Molecular weight: 311.39; LC-MS (Pos, m/z)=312.16[M+H]⁺.

### Example 5: Synthesis of 6-isopropyl-4-(4-methoxy-4-methylpiperidin-1-yl)-2-oxo-1,2-dihydroquinoline-3-carbonitrile (Compound 179)

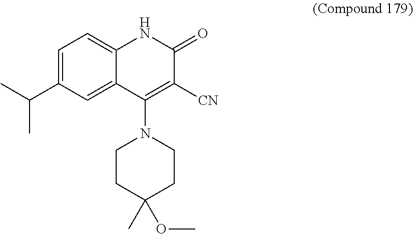 [SMILES: COC1(C)CCN(c2c(C#N)c(=O)[nH]c3ccc(C(C)C)cc23)CC1]

### Step 1: Synthesis of 4-(4-methoxy-4-methylpiperidin-1-yl)-2-oxo-6-(prop-1-en-2-yl)-1,2-dihydroquinoline-3-carbonitrile

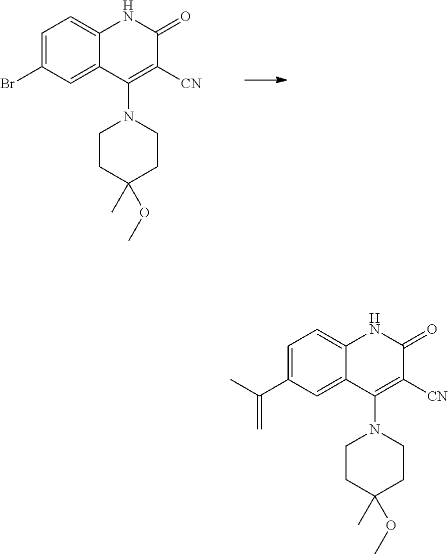 [SMILES: COC1(C)CCN(c2c(C#N)c(=O)[nH]c3ccc(Br)cc23)CC1 ; C=C(C)c1ccc2[nH]c(=O)c(C#N)c(N3CCC(C)(OC)CC3)c2c1]

The product 6-bromo-4-(4-methoxy-4-methylpiperidin-1-yl)-2-oxo-1,2-dihydroquinoline-3-carbonitrile (500 mg, 1.33 mmol, 1.0 eq) obtained in step 6 of Example 1 was dissolved in a mixed solvent of 1,4-dioxane (5 mL) and H₂O (1 mL), and potassium trifluoro(prop-1-en-2-yl)borate (295 mg, 2.0 mmol, 1.5 eq) and cesium carbonate (1.3 g, 4.0 mmol, 3.0 eq) were added. After purge with nitrogen, [1,1′-bis(diphenylphosphino)ferrocene]palladium dichloride (97 mg, 0.13 mmol, 0.1 eq) was added. Then the reaction mixture was reacted at 100° C. for 12 h under nitrogen atmosphere. After the reaction completion as detected by LC-MS, the reaction mixture was added with water (10 mL) and extracted with dichloromethane (10 mL×3). The organic phases were combined, dried over anhydrous sodium sulfate and filtered, and the filtrate was concentrated under reduced pressure to give a crude product, which was purified by silica gel column chromatography (DCM:MeOH=60:1) to give the product (400 mg, yield: 89%).

### Step 2: Synthesis of 6-isopropyl-4-(4-methoxy-4-methylpiperidin-1-yl)-2-oxo-1,2-dihydroquinoline-3-carbonitrile

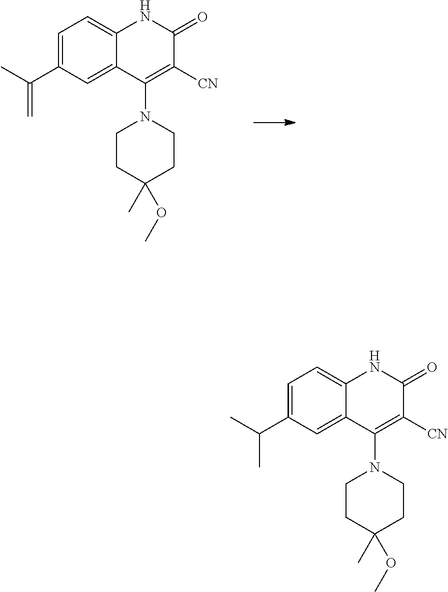 [SMILES: C=C(C)c1ccc2[nH]c(=O)c(C#N)c(N3CCC(C)(OC)CC3)c2c1 ; COC1(C)CCN(c2c(C#N)c(=O)[nH]c3ccc(C(C)C)cc23)CC1]

Intermediate 4-(4-methoxy-4-methylpiperidin-1-yl)-2-oxo-6-(prop-1-en-2-yl)-1,2-dihydroquinoline-3-carbonitrile (400 mg, 1.18 mmol, 1.0 eq) was dissolved in methanol (5 mL), and Pd/C (40 mg) was added, and the reaction mixture was reacted for 12 h under hydrogen atmosphere. After the reaction completion as detected by LC-MS, the reaction mixture was filtered under vacuum and concentrated under reduced pressure to give a crude product, which was slurried with methyl tert-butyl ether to give the product (300 mg, yield: 75%).

¹HNMR (400 MHz, DMSO-d₆) δ (ppm): 11.68 (s, 1H), 7.50-7.51 (d, 2H), 7.24-7.24 (d, 1H), 3.51-3.61 (m, 4H), 3.18 (s, 3H), 2.95-2.97 (m, 1H), 1.89-1.93 (d, 2H), 1.75-1.80 (m, 2H), 1.22-1.23 (m, 9H).

Molecular formula: C₂₀H₂₅N₃O₂; Molecular weight: 339.44; LC-MS (Pos, m/z)=340.19[M+H]⁺.

### Example 6: Synthesis of 6-(2-hydroxypropan-2-yl)-4-(4-methoxy-4-methylpiperidin-1-yl)-2-oxo-1,2-dihydroquinoline-3-carbonitrile (Compound 109)

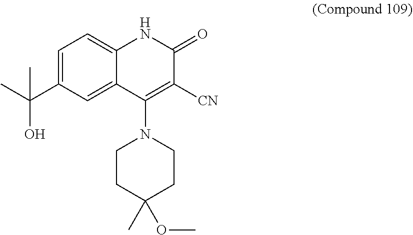 [SMILES: COC1(C)CCN(c2c(C#N)c(=O)[nH]c3ccc(C(C)(C)O)cc23)CC1]

### Step 1: Synthesis of 6-(1,2-dihydroxyethyl)-4-(4-methoxy-4-methylpiperidin-1-yl)-2-oxo-1,2-dihydroquinoline-3-carbonitrile

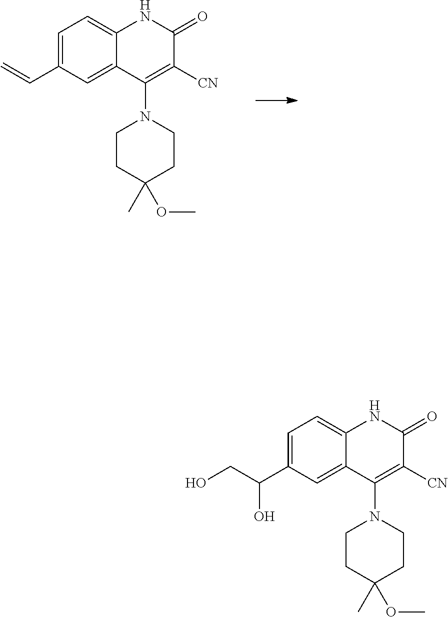 [SMILES: C=Cc1ccc2[nH]c(=O)c(C#N)c(N3CCC(C)(OC)CC3)c2c1 ; COC1(C)CCN(c2c(C#N)c(=O)[nH]c3ccc(C(O)CO)cc23)CC1]

The product 4-(4-methoxy-4-methylpiperidin-1-yl)-2-oxo-6-vinyl-1,2-dihydroquinoline-3-carbonitrile (14 g, 0.043 mol, 1.0 eq) obtained in step 1 of Example 3 was dissolved in a mixed solvent of tert-butanol (280 mL) and water (280 mL), and methanesulfonamide (4.11 g, 0.043 mol, 1.0 eq) and AD-mix (168 g) were added. Then the reaction mixture was reacted at room temperature for 12 h. After the reaction completion as detected by LC-MS, the reaction mixture was directly used in the next step without any treatment.

### Step 2: Synthesis of 6-formyl-4-(4-methoxy-4-methylpiperidin-1-yl)-2-oxo-1,2-dihydroquinoline-3-carbonitrile

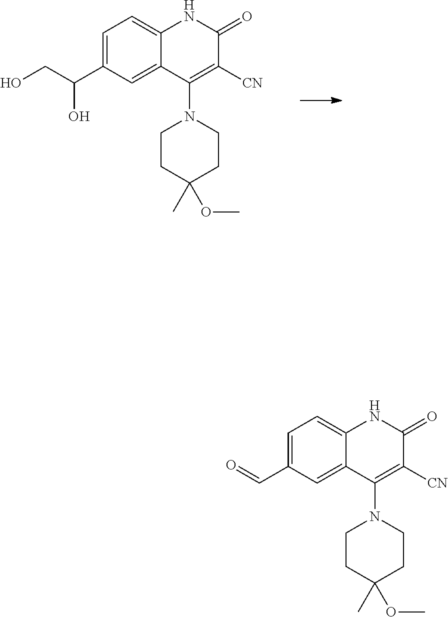 [SMILES: COC1(C)CCN(c2c(C#N)c(=O)[nH]c3ccc(C(O)CO)cc23)CC1 ; COC1(C)CCN(c2c(C#N)c(=O)[nH]c3ccc(C=O)cc23)CC1]

Tetrahydrofuran (200 mL) was added to the reaction flask in the previous step, and then sodium periodate (36.9 g, 0.17 mol, 4.0 eq) was added. Then the reaction mixture was reacted for 24 h. After the reaction completion as detected by LC-MS, the reaction mixture was added with dichloromethane (200 mL), stirred for 30 min and filtered, and liquid separation was performed. The aqueous phase was extracted with dichloromethane (200 mL×2), and the organic phases were combined, dried over anhydrous sodium sulfate, filtered and concentrated under reduced pressure to give a crude product, which was purified by silica gel column chromatography (DCM:MeOH=100:1) to give the product (5.9 g, two-step yield: 42%).

### Step 3: Synthesis of 6-(1-hydroxyethyl)-4-(4-methoxy-4-methylpiperidin-1-yl)-2-oxo-1, 2-dihydroquinoline-3-carbonitrile

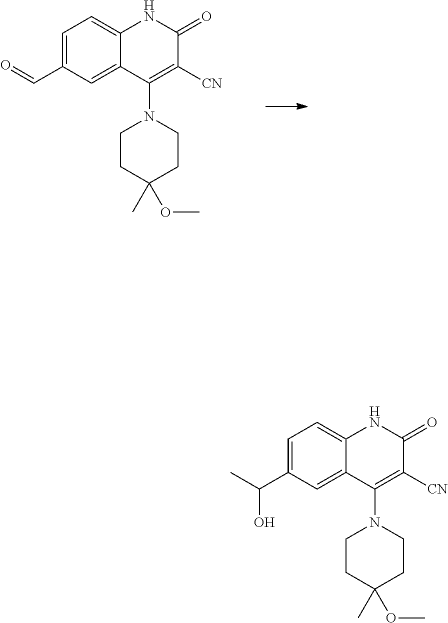 [SMILES: COC1(C)CCN(c2c(C#N)c(=O)[nH]c3ccc(C=O)cc23)CC1 ; COC1(C)CCN(c2c(C#N)c(=O)[nH]c3ccc(C(C)O)cc23)CC1]

Intermediate 6-formyl-4-(4-methoxy-4-methylpiperidin-1-yl)-2-oxo-1,2-dihydroquinoline-3-carbonitrile (4 g, 12.3 mmol, 1.0 eq) was dissolved in tetrahydrofuran (40 mL), and then the reaction mixture was cooled to −30° C., added with methylmagnesium chloride (12.4 mL, 36.9 mmol, 3.0 eq) and reacted for 5 h. After the reaction completion as detected by LC-MS, the reaction mixture was added with water (40 mL) and extracted with ethyl acetate (50 mL×3). The organic phases were combined, dried over anhydrous sodium sulfate, and filtered under vacuum. The filtrate was concentrated under reduced pressure to give a crude product, which was purified by silica gel column chromatography (DCM:MeOH=50:1) to give the product (2.28 g, yield: 54%).

### Step 4: Synthesis of 6-acetyl-4-(4-methoxy-4-methylpiperidin-1-yl)-2-oxo-1,2-dihydroquinoline-3-carbonitrile

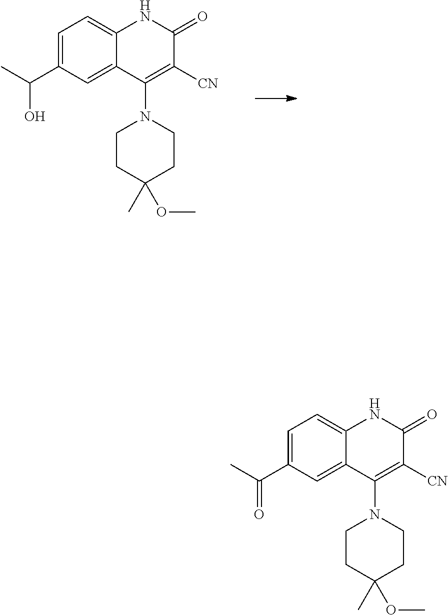 [SMILES: COC1(C)CCN(c2c(C#N)c(=O)[nH]c3ccc(C(C)O)cc23)CC1 ; COC1(C)CCN(c2c(C#N)c(=O)[nH]c3ccc(C(C)=O)cc23)CC1]

Intermediate 6-(1-hydroxyethyl)-4-(4-methoxy-4-methylpiperidin-1-yl)-2-oxo-1, 2-dihydroquinoline-3-carbonitrile (2.2 g, 6.44 mmol, 1.0 eq) was dissolved in dichloromethane (40 mL), and then the reaction mixture was cooled to 0° C., added with Dess-Martin oxidant (5.47 g, 12.88 mmol, 2.0 eq) and reacted for 3 h. After the reaction completion as detected by LC-MS, the reaction mixture was filtered under vacuum, and the filtrate was concentrated under reduced pressure to give a crude product, which was purified by silica gel column chromatography (DCM:MeOH=80:1) to give the product (1.9 g, yield: 87%).

### Step 5: Synthesis of 6-(2-hydroxypropan-2-yl)-4-(4-methoxy-4-methylpiperidin-1-yl)-2-oxo-1,2-dihydroquinoline-3-carbonitrile

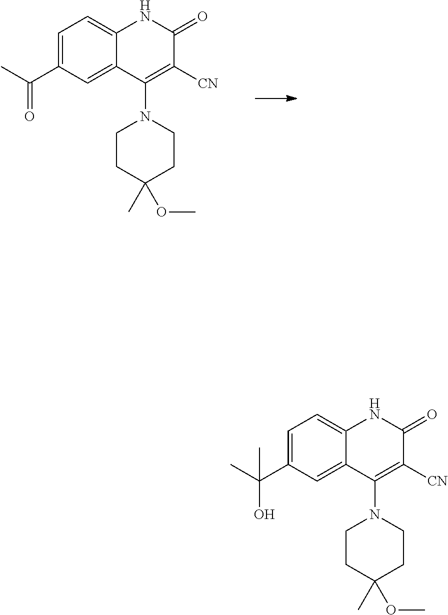 [SMILES: COC1(C)CCN(c2c(C#N)c(=O)[nH]c3ccc(C(C)=O)cc23)CC1 ; COC1(C)CCN(c2c(C#N)c(=O)[nH]c3ccc(C(C)(C)O)cc23)CC1]

Intermediate 6-acetyl-4-(4-methoxy-4-methylpiperidin-1-yl)-2-oxo-1,2-dihydroquinoline-3-carbonitrile (1.9 g, 5.6 mmol, 1.0 eq) was dissolved in tetrahydrofuran (40 mL), and then the reaction mixture was cooled to −30° C., added with methylmagnesium bromide (5.6 mL, 16.8 mmol, 3.0 eq) and reacted for 3 h. After the reaction completion as detected by LC-MS, the reaction mixture was added with water (30 mL) and extracted with ethyl acetate (10 mL×3). The organic phases were combined, dried over anhydrous sodium sulfate and filtered, and the filtrate was concentrated under reduced pressure to give a crude product, which was purified by silica gel column chromatography (DCM:MeOH=60:1) to give the product (1.52 g, yield: 76%).

¹HNMR (400 MHz, DMSO-d₆) δ (ppm): 11.69 (s, 1H), 7.85-7.86 (d, 1H), 7.65-7.68 (m, 1H), 7.22-7.24 (d, 1H), 5.16 (s, 1H), 3.53-3.62 (m, 4H), 3.19 (s, 3H), 1.90-1.94 (d, 2H), 1.73-1.80 (m, 2H), 1.45 (s, 6H), 1.23 (s, 3H).

Molecular formula: C₂₀H₂₅N₃O₃; Molecular weight: 355.44; LC-MS (Pos, m/z)=356.19[M+H]⁺.

### Example 7: Synthesis of 6-(cyclopropyl(hydroxy)methyl)-4-(4-methoxy-4-methylpiperidin-1-yl)-2-oxo-1,2-dihydroquinoline-3-carbonitrile (Compound 130)

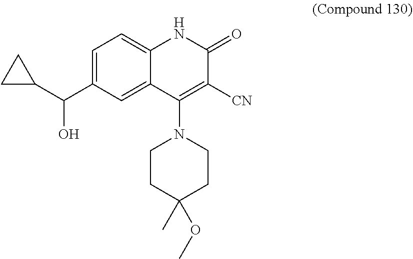 [SMILES: COC1(C)CCN(c2c(C#N)c(=O)[nH]c3ccc(C(O)C4CC4)cc23)CC1]

### Step 1: Synthesis of 6-(cyclopropyl(hydroxy)methyl)-4-(4-methoxy-4-methylpiperidin-1-yl)-2-oxo-1,2-dihydroquinoline-3-carbonitrile

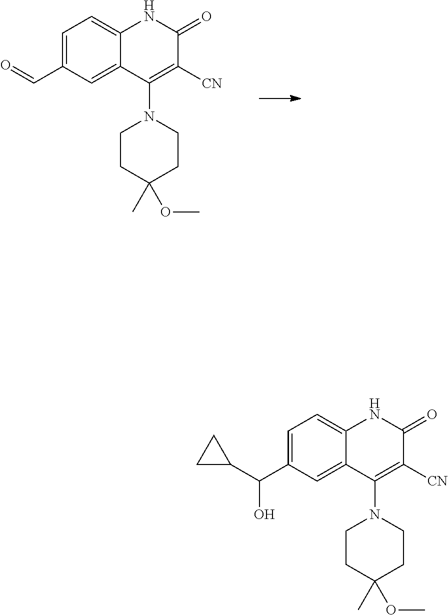 [SMILES: COC1(C)CCN(c2c(C#N)c(=O)[nH]c3ccc(C=O)cc23)CC1 ; COC1(C)CCN(c2c(C#N)c(=O)[nH]c3ccc(C(O)C4CC4)cc23)CC1]

The product 6-formyl-4-(4-methoxy-4-methylpiperidin-1-yl)-2-oxo-1,2-dihydroquinoline-3-carbonitrile (500 mg, 1.54 mmol, 1.0 eq) obtained in step 2 of Example 6 was dissolved in tetrahydrofuran (10 mL), and cyclopropyl magnesium bromide (2.3 mL, 2.3 mmol, 1.5 eq) was added dropwise at 30° C., and then the reaction mixture was reacted for 3 h. After the reaction completion as detected by LC-MS, the reaction mixture was added with water (10 mL) and extracted with dichloromethane (10 mL×3). The organic phases were combined, dried over anhydrous sodium sulfate and filtered, and the filtrate was concentrated under reduced pressure to give a crude product, which was first purified by silica gel column chromatography (DCM:MeOH=50:1) and then slurried with methyl tert-butyl ether (10 mL) to give the product (300 mg, yield: 53%).

¹HNMR (400 MHz, DMSO-d₆) δ (ppm): 11.71 (s, 1H), 7.74 (s, 1H), 7.60-7.63 (d, 1H), 7.24-7.26 (m, 1H), 5.27-5.28 (d, 1H), 4.04-4.06 (m, 1H), 3.55-3.57 (m, 4H), 3.19 (s, 3H), 1.89-1.92 (d, 2H), 1.75-1.79 (d, 2H), 1.23 (s, 3H), 1.01-1.05 (s, 1H), 0.45-0.48 (d, 4H).

Molecular formula: C₂₁H₂₅N₃O₃; Molecular weight: 367.45; LC-MS (Pos, m/z)=368.19[M+H]⁺.

### Example 8: Synthesis of 4-(4-methoxy-4-methylpiperidin-1-yl)-6-(2-methoxyethoxy)-2-oxo-1,2-dihydroquinoline-3-carbonitrile (Compound 145)

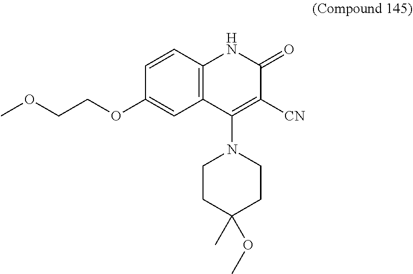 [SMILES: COCCOc1ccc2[nH]c(=O)c(C#N)c(N3CCC(C)(OC)CC3)c2c1]

### Step 1: Synthesis of 2-methoxyethyl 5-(2-methoxyethoxy)-2-nitrobenzoate

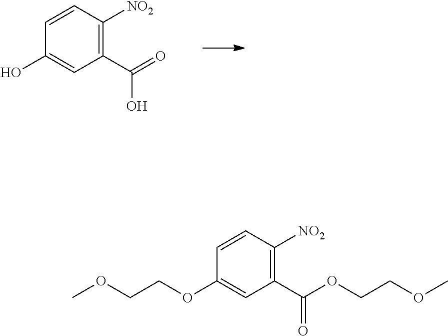

The starting material 5-hydroxy-2-nitrobenzoic acid (5.0 g, 27.31 mmol, 1.0 eq) was dissolved in DMF (50 mL), and anhydrous potassium carbonate (15 g, 109.21 mmol, 4.0 eq) and 1-bromo-2-methoxyethane (11.4 g, 82.02 mmol, 3 eq) were added, and the reaction mixture was reacted at 60° C. for 23 h. After the reaction completion as detected by LC-MS, the reaction mixture was cooled to room temperature, added with water (200 mL), and extracted with ethyl acetate (200 mL×2). The organic phases were combined and washed with water (100 mL×2), and liquid separation was performed. The resulting organic phase was dried over anhydrous sodium sulfate, filtered and concentrated under reduced pressure to give the product (4.0 g, yield: 49%).

### Step 2: Synthesis of 2-methoxyethyl 2-amino-5-(2-methoxyethoxy)benzoate

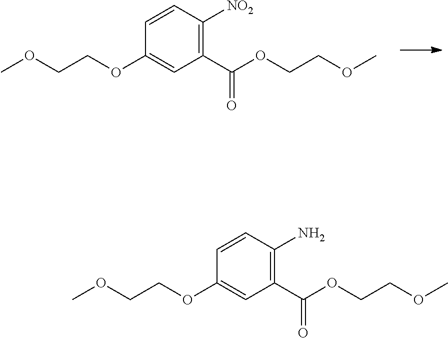

Intermediate 2-methoxyethyl 5-(2-methoxyethoxy)-2-nitrobenzoate (4.0 g, 13.36 mmol, 1.0 eq) was dissolved in absolute methanol (100 mL), and 10% palladium on carbon (1 g) was added. After purge with hydrogen for three times, the reaction mixture was reacted at room temperature for 20 h. After the reaction completion as detected by LC-MS, the reaction mixture was filtered. The filter cake was rinsed with methanol, and the filtrate was concentrated under reduced pressure to give the product (5 g of crude product), which was used in the next step according to a theoretical amount.

### Step 3: Synthesis of 2-methoxyethyl 2-(2-cyanoacetamido)-5-(2-methoxyethoxy)benzoate

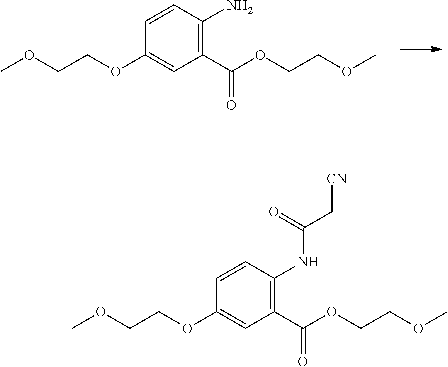

Intermediate 2-methoxyethyl 2-amino-5-(2-methoxyethoxy)benzoate (5 g of crude product, 13.36 mmol) was dissolved in dichloromethane (60 mL), and 1-(3-dimethylaminopropyl)-3-ethylcarbodiimide hydrochloride (3.8 g, 20 mmol, 1.5 eq) and cyanoacetic acid (1.36 g, 16 mmol, 1.2 eq) were added. After the addition, the reaction mixture was stirred at room temperature for 1 h. After starting material disappearance as detected by TLC, the reaction mixture was added with dichloromethane (100 mL) and washed with water (50 mL×2), and the liquid separation was performed. The organic phase was dried over anhydrous magnesium sulfate and filtered, and the filtrate was concentrated under reduced pressure to give the product (4.78 g of crude product), which was used in the next step according to a theoretical amount.

### Step 4: Synthesis of 4-hydroxy-6-(2-methoxyethoxy)-2-oxo-1,2-dihydroquinoline-3-carbonitrile

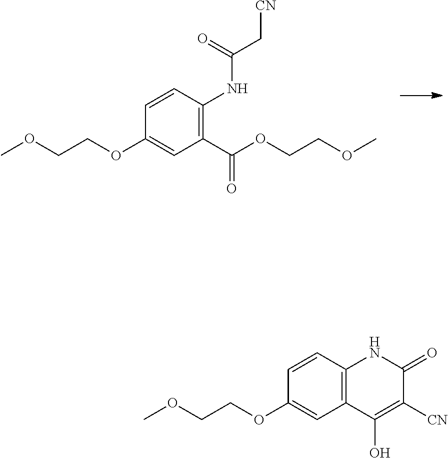

Intermediate 2-methoxyethyl 2-(2-cyanoacetamido)-5-(2-methoxyethoxy)benzoate (4.5 g, 13.36 mmol, 1.0 eq) was dissolved in absolute ethanol (150 mL), and then the reaction mixture was heated to 50° C. for dissolution, added with sodium ethoxide (2.7 g, 40.08 mmol, 3.0 eq) and stirred at 50° C. for 1 h. After starting material disappearance as detected by TLC, the reaction mixture was concentrated under reduced pressure, added with water (50 mL), and adjusted to pH=2 with concentrated hydrochloric acid, and solids were precipitated. The reaction mixture was then filtered, and the filter cake was washed successively with water and acetone, and then dried to give the product (3.2 g, three-step yield: 92.2%).

### Step 5: Synthesis of 2, 4-dichloro-6-(2-methoxyethoxy)-1,2-dihydroquinoline-3-carbonitrile

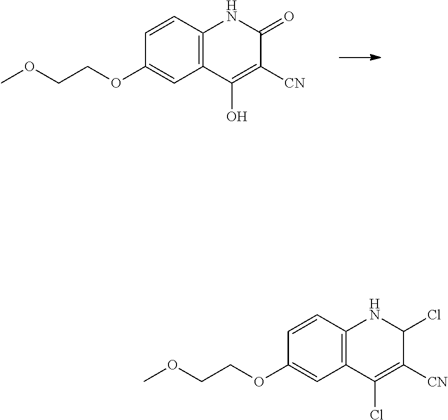 [SMILES: COCCOc1ccc2[nH]c(=O)c(C#N)c(O)c2c1 ; COCCOc1ccc2c(c1)C(Cl)=C(C#N)C(Cl)N2]

Intermediate 4-hydroxy-6-(2-methoxyethoxy)-2-oxo-1,2-dihydroquinoline-3-carbonitrile (3.2 g, 12.30 mmol, 1.0 eq) was dissolved in anhydrous acetonitrile (150 mL), and phosphorus pentachloride (5.1 g, 24.60 mmol, 2.0 eq) and phosphorus oxychloride (6.6 g, 43.05 mmol, 3.5 eq) were added. After the addition, the reaction mixture was heated to 80° C. and reacted for 3 h. After the presence of a small amount of remaining starting material as detected by LC-MS, the reaction mixture was cooled to room temperature, poured into ice water (200 mL) and stirred for 10 min, and yellow solids were precipitated. The reaction mixture was then filtered, and the filter cake was rinsed with water to give the product (5.4 g of crude product), which was used in the next step according to a theoretical amount.

### Step 6: Synthesis of 4-chloro-6-(2-methoxyethoxy)-2-oxo-1,2-dihydroquinoline-3-carbonitrile

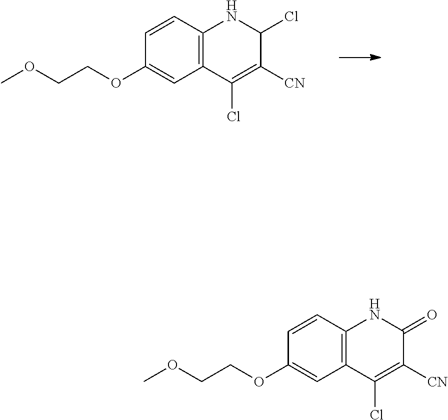 [SMILES: COCCOc1ccc2c(c1)C(Cl)=C(C#N)C(Cl)N2 ; COCCOc1ccc2[nH]c(=O)c(C#N)c(Cl)c2c1]

Intermediate 2,4-dichloro-6-(2-methoxyethoxy)-1,2-dihydroquinoline-3-carbonitrile (3.6 g, 12.30 mmol, 1.0 eq) was dissolved in a mixed solvent of trifluoroacetic acid (36 mL) and water (9 mL), and the reaction mixture was heated to 90° C., reacted for 4 h, and then stirred at room temperature for 14 h. Then the reaction mixture was poured into water (100 mL), and yellow solids were precipitated. The reaction mixture was then filtered, and the filter cake was washed successively with water and acetone, and then dried to give the product (2.39 g, yield: 69.8%).

### Step 7: Synthesis of 4-(4-methoxy-4-methylpiperidin-1-yl)-6-(2-methoxyethoxy)-2-oxo-1, 2-dihydroquinoline-3-carbonitrile

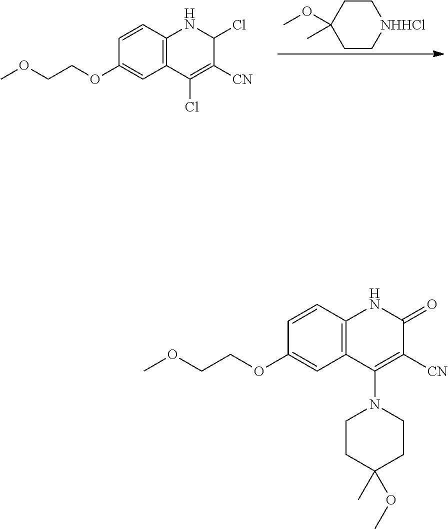

Intermediate 4-chloro-6-(2-methoxyethoxy)-2-oxo-1,2-dihydroquinoline-3-carbonitrile (500 mg, 1.79 mmol, 1.0 eq) was dissolved in N,N-dimethylacetamide (5 mL), and DIPEA (926.8 mg, 7.18 mmol, 4.0 eq) and 4-methoxy-4-methylpiperidine hydrochloride (415.1 mg, 2.51 mmol, 1.4 eq) were added. Then the reaction mixture was heated to 80° C. and reacted for 1 h. After the starting material disappearance as detected by LC-MS, the reaction mixture was cooled to room temperature, poured into ice water (50 mL), and extracted with ethyl acetate (50 mL×3). The organic phases were combined, washed with water (50 mL×2), dried over anhydrous magnesium sulfate and filtered, and the filtrate was concentrated under reduced pressure to give a pale yellow solid, which was added to a mixed solvent of ethyl acetate (2 mL) and methyl tert-butyl ether (8 mL), slurried at 50° C. for 0.5 h, and filtered under vacuum while hot. The resulting filter cake was dried to give the product (369.3 mg, yield: 55.5%).

¹HNMR (400 MHz, DMSO-d₆) δ (ppm): 11.70 (s, 1H), 7.31-7.28 (d, 1H), 7.25-7.23 (d, 1H), 7.11-7.10 (s, 1H), 4.17-4.14 (m, 2H), 3.70-3.68 (m, 2H), 3.60-3.50 (m, 4H), 3.32 (s, 3H), 3.18 (s, 3H), 1.92-1.86 (m, 2H), 1.81-1.75 (m, 2H), 1.22 (s, 3H).

Molecular formula: C₂₀H₂₅N₃O₄; Molecular weight: 371.44; LC-MS (Pos, m/z)=372.17[M+H]⁺.

The present invention can be better understood according to the following experimental examples. However, it is easily understood by those skilled in the art that the contents described in experimental examples are only used to illustrate the present invention, and should not and will not limit the present invention described in detail in the claims.

### Experimental Example 1: PDE9 Enzymatic Evaluation

Test samples: the compounds disclosed herein, prepared according to the corresponding examples of the present invention.

### 1. Experimental Materials and Instruments

PDE9A2 enzyme (BPS, Cat. No. 60090)
384 well plate (Perkin Elmer, Cat. No. 6007279)

### 2. Experimental Procedures

Preparation of the compounds: Compounds were formulated into 10 mM compound stock solution in DMSO for long term storage. The obtained compound stock solution was 100-fold diluted with DMSO to give 100 μM compound mother liquor, which was 3-fold diluted with DMSO to give 8-10 concentration gradients of diluted compound mother liquor (100×).

Incubation with compound: Diluted compound mother liquor was pipetted into a 384-well plate using an ultramicro-liquid pipetting system (Echo); 200 nL of the diluted compound mother liquor and 10 μL of PDE9A2 enzyme solution were added to each compound well; after centrifugation at 1000 rpm for 1 min, the mixtures were incubated at room temperature for 15 min. Subsequently, 10 μL of the substrate mixture was added, and after centrifugation at 1000 rpm for 1 min, the resulting mixture was incubated at room temperature with oscillating for 30 min. Finally, stop solution was added to end the reaction, and the resulting reaction system was incubated at room temperature with oscillating for 60 min. In the maximum reading well (Max), the compound was replaced by solvent; in the minimum reading well (Min), the compound and the enzyme solution were replaced by solvent.

Detection: the fluorescence readings (F) at 480 nm/535 nm were detected using a microplate reader.

Calculation: The inhibition rate was calculated according to the following formula and IC₅₀ was fitted using GraphPad Prism 5.0:

$$Inhibition rate ( \% ) = \frac{FMax - Fcomp o u n d}{FMax - FMin} \times 100 \%$$

3. The experiment results are shown in Table 2 below:

TABLE 2

| Test samples | PDE9A2 IC₅₀ (nM) |
|---|---|
| Compound 96 | 5 |
| Compound 107 | 8 |
| Compound 109 | 15 |
| Compound 130 | 2 |
| Compound 145 | 12 |
| Compound 158 | 12 |
| Compound 179 | 18 |
| Compound 180 | 8 |

The compounds disclosed herein have very good PDE9 enzymatic inhibition activity and thus have potential value for clinical application.

### Experimental Example 2: Evaluation of Liver Microsomal Stability of Compounds Disclosed Herein

Test samples: the compounds disclosed herein and the compound I-8 of international patent application WO2017019723A1 (prepared by referring to the examples for synthesis in WO2017019723A1) having the following structural formula:

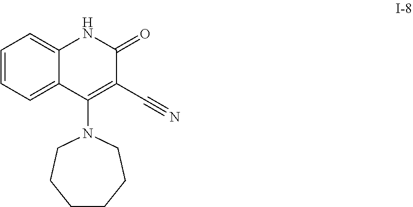 [SMILES: N#Cc1c(N2CCCCCC2)c2ccccc2[nH]c1=O]

### Composition of the Incubation System:

| Substances | Initial | | Proportion | Final | |
|---|---|---|---|---|---|
| to be added | concentration | | (%) | concentration | |
| Phosphate buffer | 100 | mM | 50 | 50 | mM |
| MgCl₂ | 20 | mM | 5 | 1 | mM |
| Liver microsome | 20 | mg | 2.5 | 0.5 | mg |
| | | protein/mL | | | protein/mL |
| Water to be | — | | 22.5 | — | |
| supplemented | | | | | |
| Compound | 10 | μM | 10 | 1 | μM |
| β-NADPH | 10 | mM | 10 | 1 | mM |

### Compound Preparation:

An appropriate amount of the compound was precisely weighed out and dissolved in DMSO to prepare a 5.0 mM stock solution. The 5.0 mM stock solution was diluted to 1.0 mM with DMSO, and then diluted with water to 10 μM compound working solution for later use (DMSO content in the reaction system: 0.1% v/v).

### Experimental Procedures:

(1) The liver microsomes (20 mg protein/mL) were taken out from a −80° C. refrigerator, pre-incubated on a 37° C. water bath thermostatic oscillator for 3 min, and thawed for use.
(2) A mixed solution of the incubation system (without compound and D-NADPH) was prepared according to “composition of the experimental incubation system” described above, and pre-incubated on a 37° C. water bath thermostatic oscillator for 2 min.
(3) Control group (without D-NADPH): 30 μL of water and 30 μL of compound working solution (10 μM) were added to 240 μL of the mixed solution of the incubation system in step (2), and the mixture was vortexed for 30 s and mixed well; the total volume of the mixture was 300 μL; the sample was duplicated. The mixture was placed on a 37° C. water bath thermostatic oscillator for incubation, and timing was started; the sampling was conducted at 0 min and 60 min.
(4) Sample group: 70 μL of R-NADPH solution (10 mM) and 70 μL of compound working solution (10 μM) were added to 560 μL of the mixed solution in step (2), and the total volume of the mixture was 700 μL; the mixture was vortexed for 30 s and mixed well; the sample was duplicated. The mixture was placed on a 37° C. water bath thermostatic oscillator for incubation, and timing was started; the sampling was conducted at 0 min, 5 min, 10 min, 20 min, 30 min and 60 min.
(5) The mixture was vortexed for 3 min, and centrifuged at 4000 rpm for 10 min.
(6) 50 μL of the supernatant was taken and added to 150 μL of water, and the resulting mixture was vortexed and mixed well before LC/MS/MS analysis.

### Data Analysis:

The half-life (t_{1/2}) and clearance (Cl) were calculated using the following first-order kinetic formula:

Cₜ=C₀*e^{-kt}

t_{1/2}=ln 2/k=0.693/k

Clᵢₙₜ=V_{d}*k

V_{d}=1/protein content in liver microsomes

Note: k denotes the slope of the logarithm of the remaining amount of a compound vs. time, V_{d} denotes apparent volume of distribution, and C₀ denotes compound concentration at 0 h.

### Results:

Experiment on Stability of the Compounds Disclosed Herein in Rat Liver Microsomes

| | CLᵢₙₜ | t_{1/2} |
|---|---|---|
| | (mL/min/mg) | (min) |
| Compound I-8 | 0.0292 | 47.5 |
| Compound 96 | 0.0072 | 193 |
| Compound 109 | 0.0030 | 462 |
| Compound 130 | 0.0082 | 169 |
| Compound 145 | 0.0108 | 128 |
| Compound 180 | 0.0242 | 57.3 |

From the above results, it can be seen that the compounds disclosed herein have a lower clearance rate in rat liver microsomes than the compounds in prior art.

1. A compound of general formula (I) or a pharmaceutically acceptable salt or an isomer thereof: 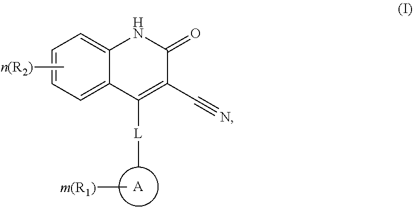
wherein
each R₂ is independently selected from hydrogen, hydroxy, amino, carboxyl, cyano, nitro, halogen, C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂aminocarbonyl, aryl, 5-6 membered heteroaryl, 4-6 membered heterocyclylcarbonyl and 5-6 membered heteroaryl-oxy, wherein the C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂aminocarbonyl, aryl, 5-6 membered heteroaryl, 4-6 membered heterocyclylcarbonyl and 5-6 membered heteroaryl-oxy are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂amino, C₁₋₆ alkylcarbonylamino, C₁₋₆ alkylsulfonylamino, C₁₋₆ alkylcarbonyloxy, C₃₋₆ cycloalkyl, C₂₋₈ alkynyl, halogenated C₁₋₆ alkyl, C₂₋₈ alkenyl, halogenated C₁₋₆ alkoxy, 4-6 membered heterocyclyl unsubstituted or optionally substituted with a substituent, and heteroaryl unsubstituted or optionally substituted with a substituent;
the substituent in the above 4-6 membered heterocyclyl optionally substituted with a substituent and heteroaryl optionally substituted with a substituent is selected from hydroxy, amino, carboxyl, cyano, nitro, halogen, C₁₋₆ alkyl and C₁₋₆ alkoxy;
L is a bond or —NH—(CH₂)t-, wherein t is 0, 1, 2 or 3;
ring A is 3-8 membered monocyclic heterocyclyl, 6-12 membered bridged heterocyclyl, 6-12 membered spiro-heterocyclyl, 6-12 membered ortho-fused heterocyclyl, aryl, 5-10 membered heteroaryl, 3-12 membered cycloalkyl or 3-12 membered cycloalkenyl, wherein the heterocyclyl has heteroatoms selected from one of or any combinations of O, S and N, the S atom may be optionally oxidized to S(O) or S(O)₂, the C atom may be optionally oxidized to C(O), and the 5-10 membered heteroaryl has heteroatoms selected from one of or any combinations of O, S and N;
each R₁ is independently selected from hydrogen, hydroxy, amino, carboxyl, cyano, nitro, halogen, C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein the C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are unsubstituted or optionally substituted with a group selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino and C₁₋₆ alkylsulfonylamino;
m and n are each independently 0, 1, 2 or 3;
when ring A is 3-8 membered monocyclic heterocyclyl, R₂ is not hydrogen;
when ring A is phenyl, L is not a bond; and
when ring A is 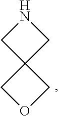
R₂ is not hydrogen.

2. The compound or the pharmaceutically acceptable salt or the isomer thereof according to claim 1, wherein
each R₂ is independently selected from hydrogen, amino, carboxyl, cyano, halogen, C₁₋₆ alkyl, C₁₋₆ alkoxy, C₁₋₆ alkylamino, (C₁₋₆ alkyl)₂amino, C₂₋₈ alkenyl, C₂₋₈ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl, wherein the C₁₋₆ alkyl, C₁₋₆ alkoxy, C₁₋₆ alkylamino, (C₁₋₆ alkyl)₂amino, C₂-8 alkenyl, C₂₋₈ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl are unsubstituted or optionally substituted with one or more groups independently selected from hydroxy, amino, cyano, halogen, C₁₋₆ alkyl, C₁₋₆ alkoxy, C₁₋₆ alkylamino, (C₁₋₆ alkyl)₂amino, C₁₋₆ alkylcarbonyloxy, C₃₋₆ cycloalkyl, and 4-6 membered heterocyclyl unsubstituted or substituted with C₁₋₆ alkyl;
L is a bond or —NH—(CH₂)t-, wherein t is 0, 1, 2 or 3;
ring A is 3-8 membered monocyclic heterocyclyl, 6-12 membered bridged heterocyclyl, 6-12 membered spiro-heterocyclyl, 6-12 membered ortho-fused heterocyclyl, phenyl or 5-membered heteroaryl, wherein the heterocyclyl has heteroatoms selected from one of or any combinations of O, S and N, the S atom may be optionally oxidized to S(O) or S(O)₂, and the C atom may be optionally oxidized to C(O);
each R₁ is independently selected from hydrogen, hydroxy, cyano, halogen, C₁₋₆ alkyl, C₁₋₆ alkoxy and 5-6 membered heteroaryl, wherein the C₁₋₆ alkyl, C₁₋₆ alkoxy and 5-6 membered heteroaryl are unsubstituted or substituted with hydroxy;
m and n are each independently 0, 1 or 2;
when ring A is 3-8 membered monocyclic heterocyclyl, R₂ is not hydrogen;
when ring A is phenyl, L is not a bond; and
when ring A is 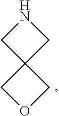
R₂ is not hydrogen.

3. The compound or the pharmaceutically acceptable salt or the isomer thereof according to claim 2, wherein
each R₂ is independently selected from halogen, C₁₋₄ alkyl, C₁₋₄ alkoxy, morpholinyl, C₂₋₆ alkenyl, C₁₋₄ alkylcarbonyl, C₁₋₄ alkylaminocarbonyl, (C₁₋₄ alkyl)₂aminocarbonyl and aminocarbonyl, wherein the C₁₋₄ alkyl, C₁₋₄ alkoxy, morpholinyl, C₂₋₆ alkenyl, C₁₋₄ alkylcarbonyl, C₁₋₄ alkylaminocarbonyl, (C₁₋₄ alkyl)₂aminocarbonyl and aminocarbonyl are unsubstituted or optionally substituted with one or more groups independently selected from hydroxy, C₁₋₄ alkyl, C₁₋₄ alkoxy, C₃₋₆ cycloalkyl, amino, C₁₋₄ alkylamino, (C₁₋₄ alkyl)₂amino, and 4-6 membered heterocyclyl unsubstituted or substituted with C₁₋₄ alkyl;
L is a bond;
ring A is 4-7 membered monocyclic heterocyclyl, wherein the 4-7 membered monocyclic heterocyclyl has heteroatoms selected from one of or combinations of two of O, S and N, and contains at least one N, ring A is connected to L via the N atom, the S atom may be optionally oxidized to S(O) or S(O)₂, and the C atom may be optionally oxidized to C(O);
preferably, ring A is selected from 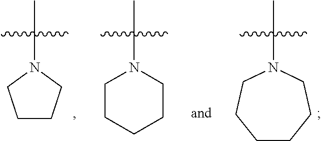
each R₁ is independently selected from hydrogen, halogen, C₁₋₄ alkyl, C₁₋₄ alkoxy, pyrazolyl, thiazolyl and triazolyl, wherein the C₁₋₄ alkyl, C₁₋₄ alkoxy, pyrazolyl, thiazolyl and triazolyl are unsubstituted or substituted with hydroxy; and
m and n are each independently 0, 1 or 2.

4. The compound or the pharmaceutically acceptable salt or the isomer thereof according to claim 3, wherein
each R₂ is independently selected from halogen, C₁₋₄ alkyl, C₁₋₄ alkoxy, C₂₋₆ alkenyl, C₁₋₄ alkylaminocarbonyl and aminocarbonyl, wherein the C₁₋₄ alkyl, C₁₋₄ alkoxy, C₂₋₆ alkenyl, C₁₋₄ alkylaminocarbonyl and aminocarbonyl are unsubstituted or optionally substituted with one or more groups independently selected from hydroxy, amino, C₁₋₄ alkyl, C₁₋₄ alkoxy, cyclopropyl, C₁₋₄ alkylamino, (C₁₋₄ alkyl)₂amino, and 4-6 membered heterocyclyl unsubstituted or substituted with C₁₋₄ alkyl;
L is a bond;
ring A is 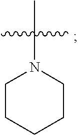 [SMILES: *N1CCCCC1]
each R₁ is independently selected from hydrogen, C₁₋₄ alkyl and C₁₋₄ alkoxy; and
m and n are each independently 0, 1 or 2.

5. The compound or the pharmaceutically acceptable salt or the isomer thereof according to claim 2, wherein
each R₂ is independently selected from amino, carboxyl, cyano, halogen, C₁₋₆ alkyl, C₁₋₆ alkoxy, C₁₋₆ alkylamino, (C₁₋₆ alkyl)₂amino, C₂₋₈ alkenyl, C₂₋₈ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl, wherein the C₁₋₆ alkyl, C₁₋₆ alkoxy, C₁₋₆ alkylamino, (C₁₋₆ alkyl)₂amino, C₂₋₈ alkenyl, C₂₋₈ alkynyl, C₁₋₆ alkylsulfonyl, C₁₋₆ alkylthio, C₃₋₆ cycloalkyl, 4-6 membered nitrogen-containing heterocyclyl, C₁₋₆ alkylcarbonyl, C₁₋₆ alkylaminocarbonyl, (C₁₋₆ alkyl)₂aminocarbonyl and aminocarbonyl are unsubstituted or optionally substituted with one or more groups independently selected from hydroxy, amino, cyano, halogen, C₁₋₆ alkyl, C₁₋₆ alkoxy, C₁₋₆ alkylamino, (C₁₋₆ alkyl)₂amino, C₁₋₆ alkylcarbonyloxy, C₃₋₆ cycloalkyl, and 4-6 membered heterocyclyl unsubstituted or substituted with C₁₋₆ alkyl;
L is a bond;
ring A is 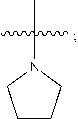 [SMILES: *N1CCCC1]
and
each R₁ is independently selected from pyrazolyl, thiazolyl and triazolyl.

6. The compound or the pharmaceutically acceptable salt or the isomer thereof according to claim 1 or 2, wherein
each R₂ is independently selected from hydrogen, amino, cyano, halogen, carboxyl, C₁₋₄ alkyl, C₁₋₄ alkoxy, C₁₋₄ alkylcarbonyl, C₂₋₆ alkynyl, C₁₋₄ alkylaminocarbonyl, (C₁₋₄ alkyl)₂aminocarbonyl, C₁₋₄ alkylthio, C₁₋₄ alkylsulfonyl, C₁₋₄ alkylamino, (C₁₋₄ alkyl)₂amino, azetidinyl, morpholinyl, piperazinyl, C₂₋₆ alkenyl and cyclopropyl, wherein the C₁₋₄ alkyl, C₁₋₄ alkoxy, C₁₋₄ alkylcarbonyl, C₂₋₆ alkynyl, C₁₋₄ alkylaminocarbonyl, (C₁₋₆ alkyl)₂aminocarbonyl, C₁₋₄ alkylthio, C₁₋₄ alkylsulfonyl, C₁₋₄ alkylamino, (C₁₋₄ alkyl)₂amino, azetidinyl, morpholinyl, piperazinyl, C₂₋₆ alkenyl and cyclopropyl are unsubstituted or optionally substituted with one or more groups independently selected from hydroxy, amino, halogen, C₁₋₄ alkyl, C₁₋₄ alkylamino, (C₁₋₄ alkyl)₂amino, cyclopropyl and C₁₋₄ alkylcarbonyloxy;
L is a bond;
ring A is 7-12 membered spiro-heterocyclyl, wherein the spiro-heterocyclyl has heteroatoms selected from one of or combinations of two of O, S and N, and contains at least one N, ring A is connected to L via the N atom, the S atom may be optionally oxidized to S(O) or S(O)₂, and the C atom may be optionally oxidized to C(O);
preferably, ring A is selected from 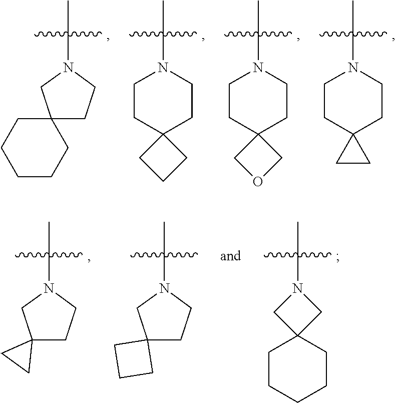
each R₁ is independently selected from hydrogen, cyano, halogen, hydroxy, and C₁₋₄ alkyl unsubstituted or substituted with hydroxy;
m and n are each independently 0, 1 or 2; and
when ring A is 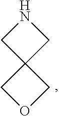
R₂ is not hydrogen.

7. The compound or the pharmaceutically acceptable salt or the isomer thereof according to claim 6, wherein
each R₂ is independently selected from hydrogen, cyano, amino, halogen, carboxyl, C₁₋₄ alkyl, C₁₋₄ alkoxy, C₁₋₄ alkylcarbonyl, C₂₋₆ alkynyl, C₁₋₄ alkylamino, (C₁₋₄ alkyl)₂amino, C₁₋₄ alkylaminocarbonyl, C₁₋₄ alkylthio, C₁₋₄ alkylsulfonyl, cyclopropyl, azetidinyl, morpholinyl and piperazinyl, wherein the C₁₋₄ alkyl, C₁₋₄ alkoxy, C₁₋₄ alkylcarbonyl, C₂₋₆ alkynyl, C₁₋₄ alkylamino, (C₁₋₄ alkyl)₂amino, C₁₋₄ alkylaminocarbonyl, C₁₋₄ alkylthio, C₁₋₄ alkylsulfonyl, cyclopropyl, azetidinyl, morpholinyl and piperazinyl are unsubstituted or optionally substituted with one or more groups independently selected from hydroxy, amino, halogen, C₁₋₄ alkyl, C₁₋₄ alkylamino, (C₁₋₄ alkyl)₂amino, cyclopropyl and C₁₋₄ alkylcarbonyloxy;
L is a bond;
ring A is selected from 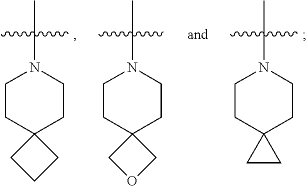
and
m and n are each independently 0, 1 or 2.

8. The compound or the pharmaceutically acceptable salt or the isomer thereof according to claim 1 or 2, wherein
L is a bond;
ring A is selected from the following groups: 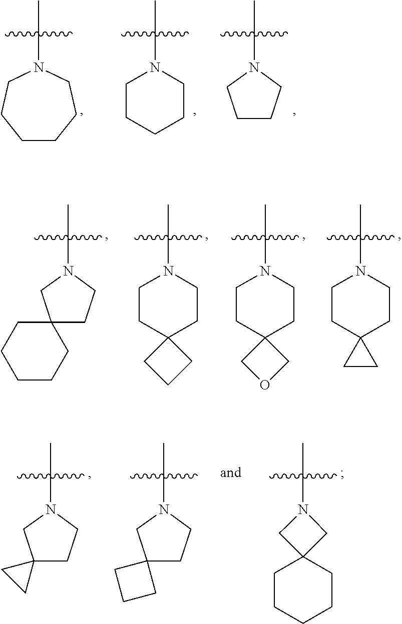
and
when ring A is 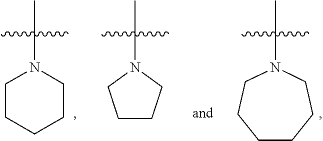
R₂ is not hydrogen.

9. The compound or the pharmaceutically acceptable salt or the isomer thereof according to claim 1, wherein the general formula (I) has a structure of general formula (II), 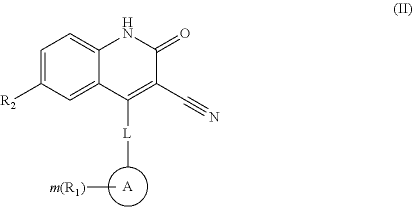
wherein R₁, R₂, L, ring A and m are described as in claim 1.

10. The compound or the pharmaceutically acceptable salt or the isomer thereof according to claim 9, wherein R₂ is halogen, C₁₋₄ alkylaminocarbonyl, C₁₋₄ alkyl optionally substituted with one or more groups independently selected from hydroxy and C₃₋₆ cycloalkyl, or C₁₋₄ alkoxy optionally substituted with C₁₋₄ alkoxy.

11. The compound or the pharmaceutically acceptable salt or the isomer thereof according to claim 10, wherein R₂ is bromine, methyl, ethyl, propyl, isopropyl, isopropyl substituted with hydroxy, methylaminocarbonyl, methyl, ethyl, propyl or butyl substituted with hydroxy and cyclopropyl, or ethoxy substituted with methoxy.

12. The compound or the pharmaceutically acceptable salt or the isomer thereof according to claim 10, wherein m(R₁) is para to a site on ring A that is substituted with L, wherein R₁ is C₁₋₄ alkyl or C₁₋₄ alkoxy, and m=2.

13. The compound or the pharmaceutically acceptable salt or the isomer thereof according to any one of claims 1-2, selected from compounds of the following structures:

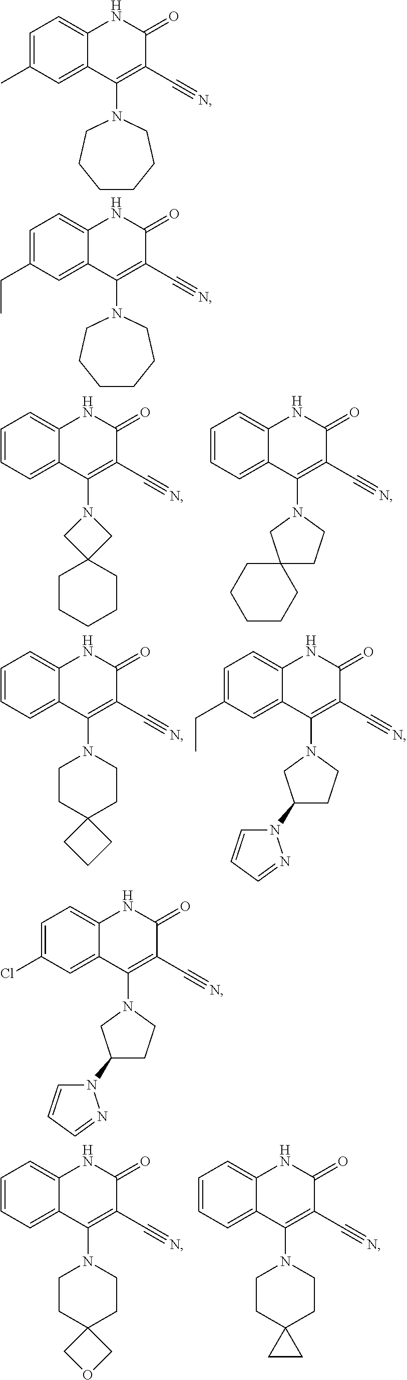

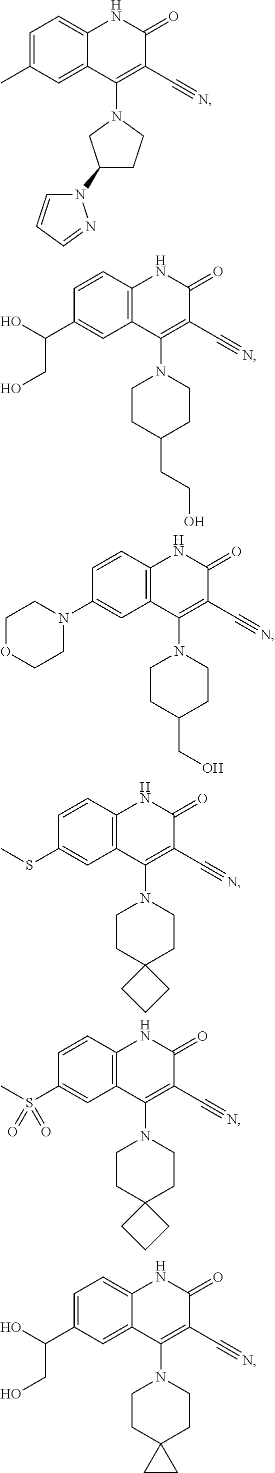

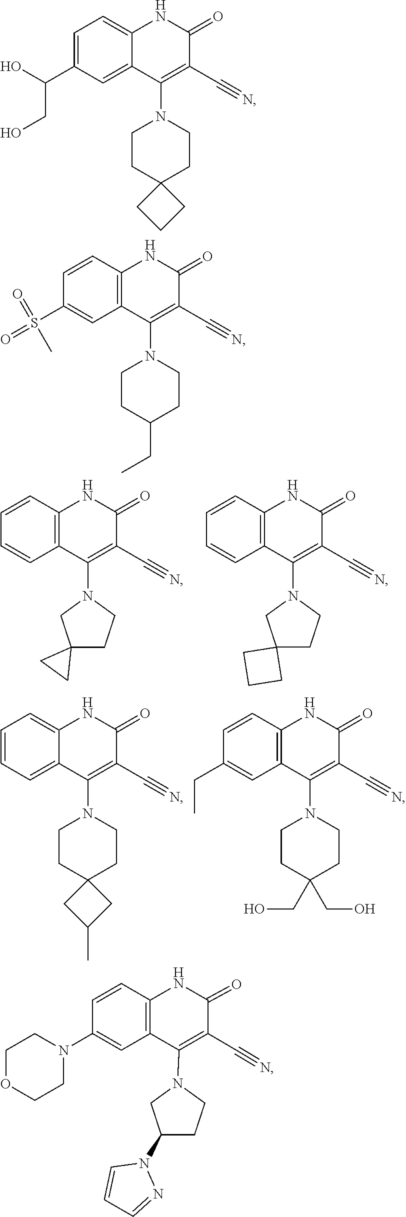

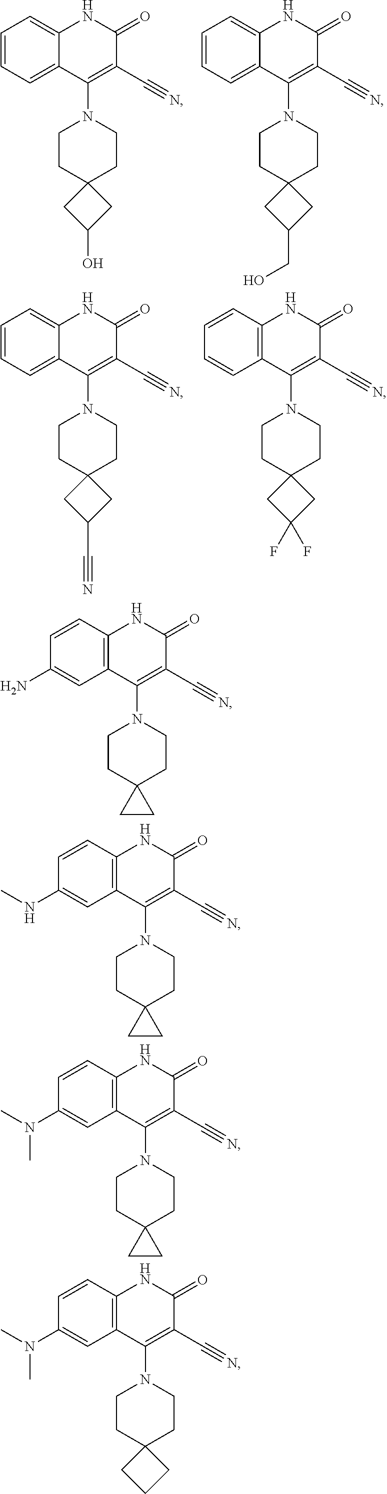

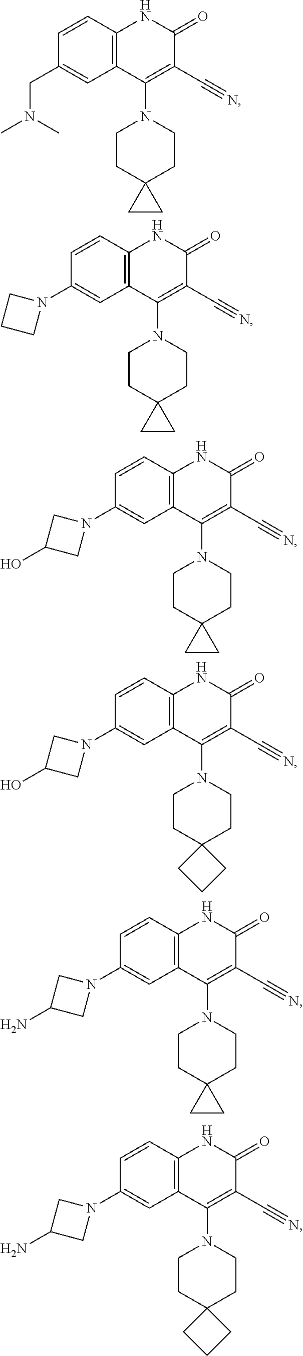

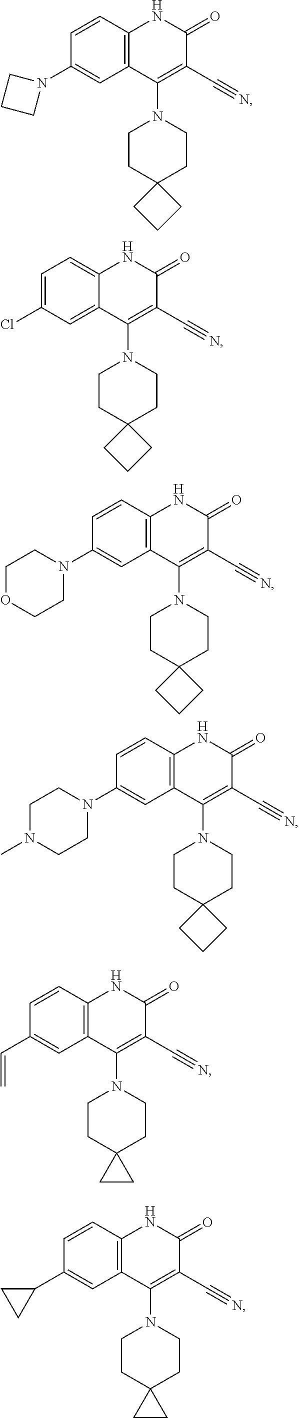

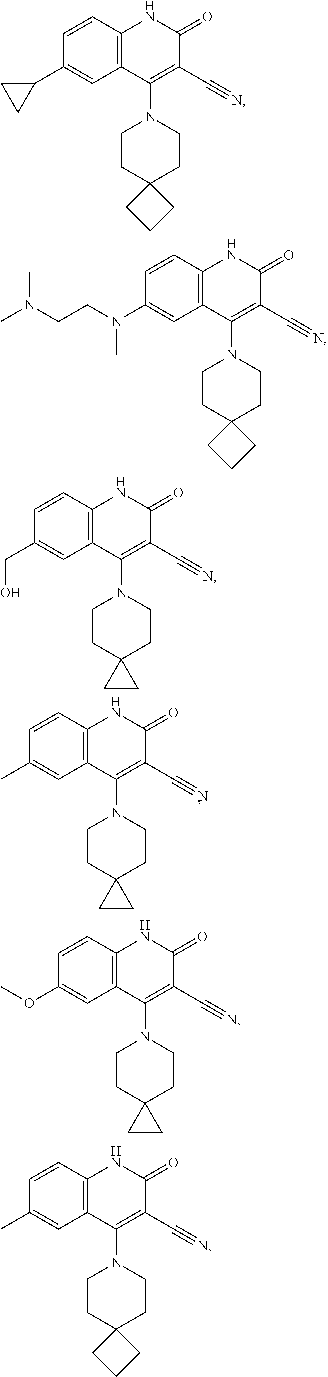

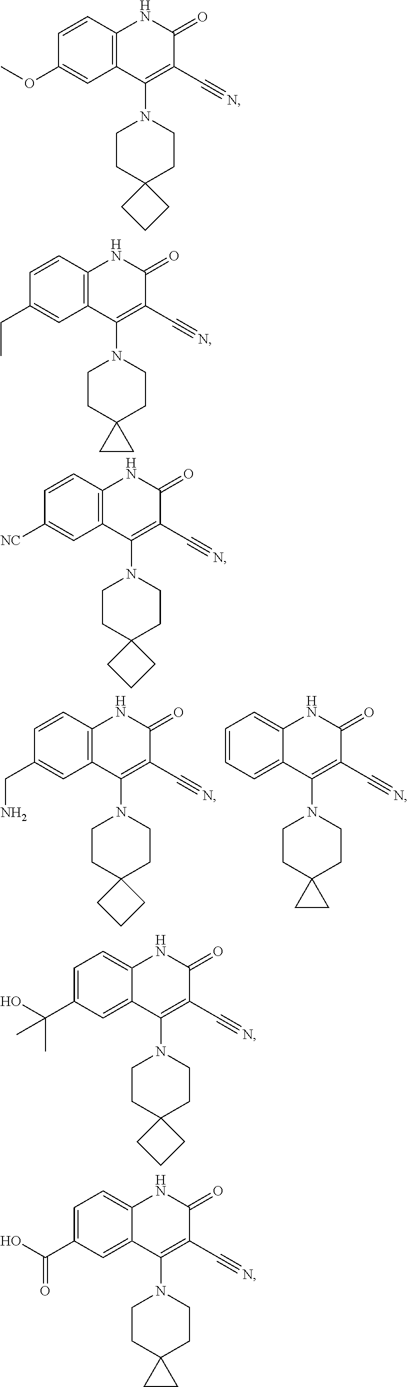

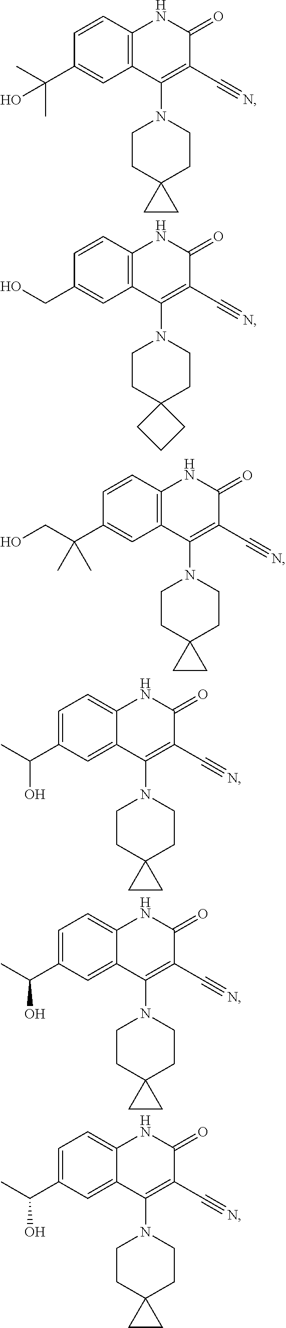

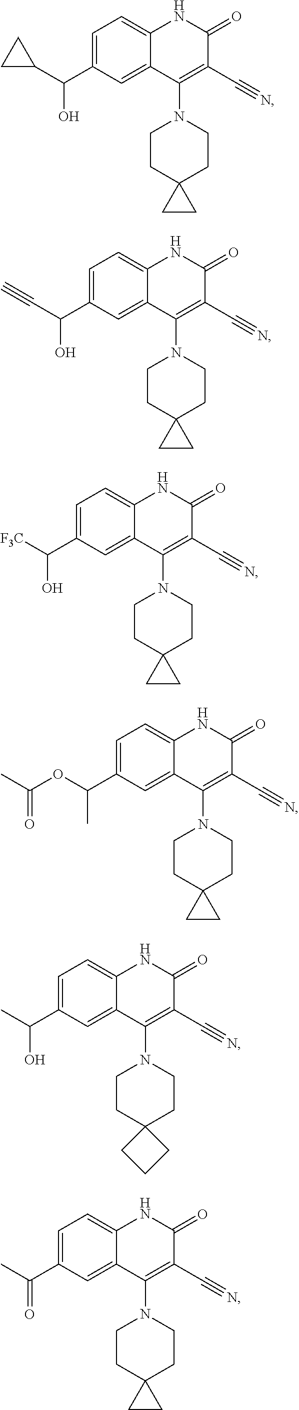

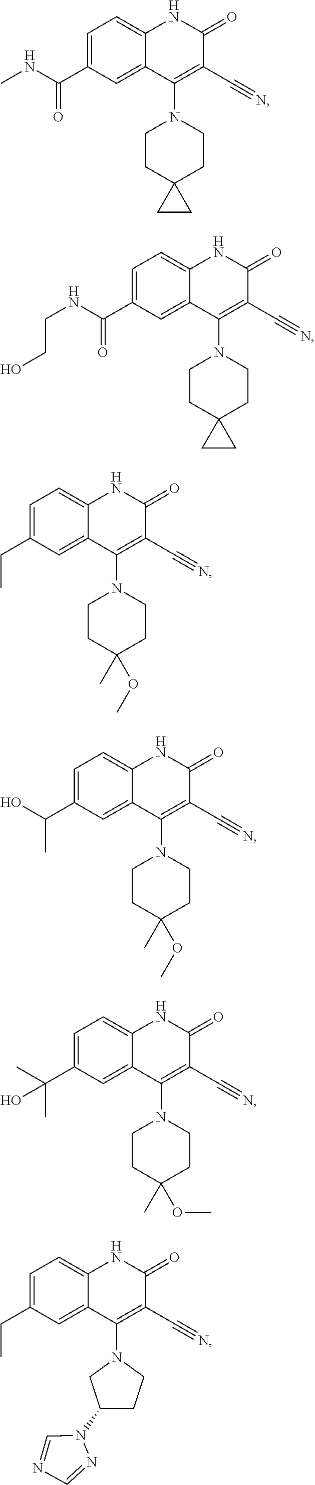

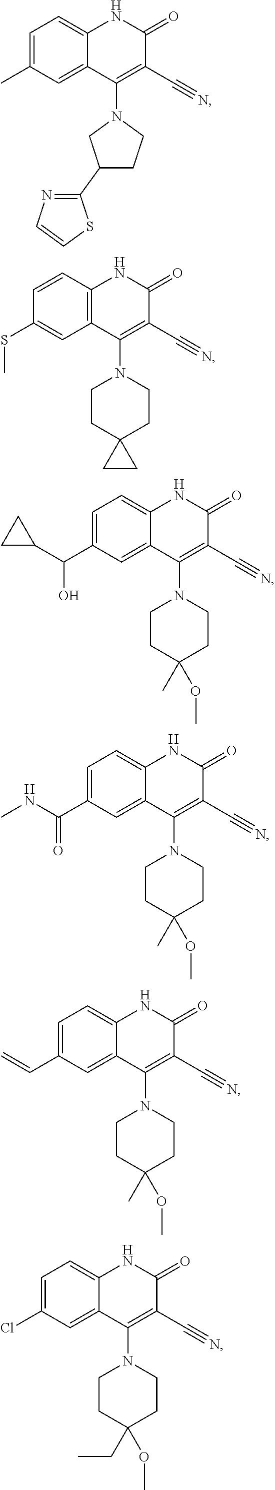

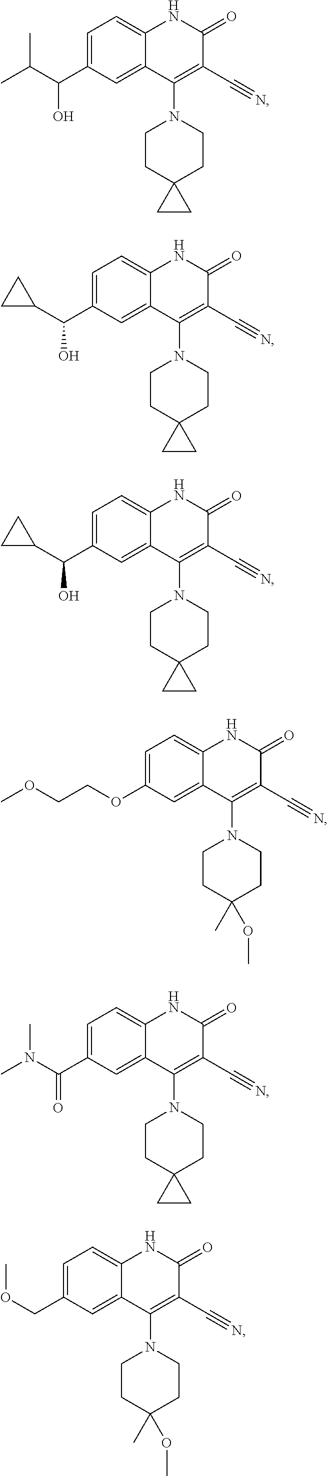

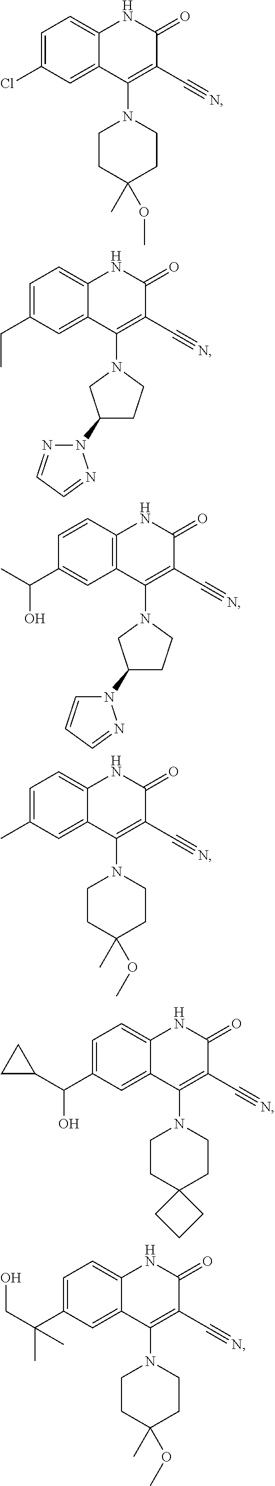

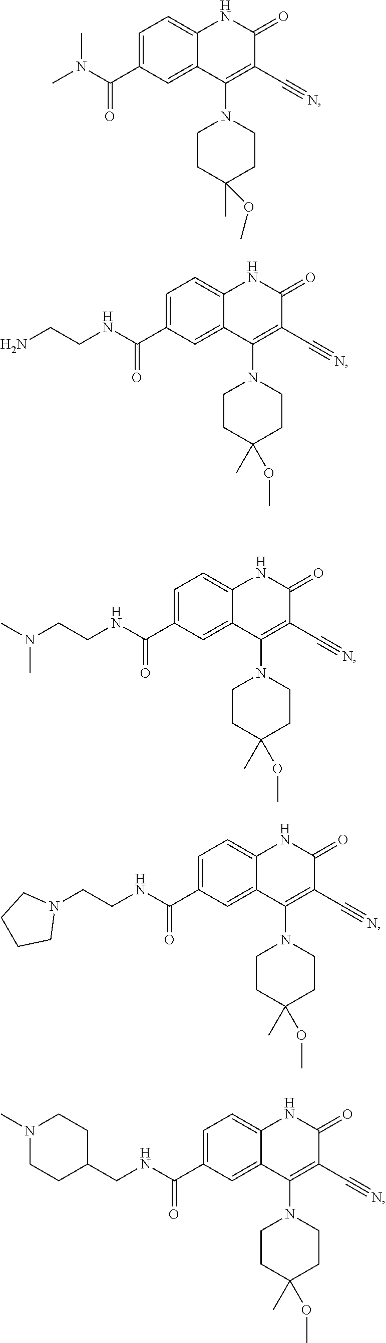

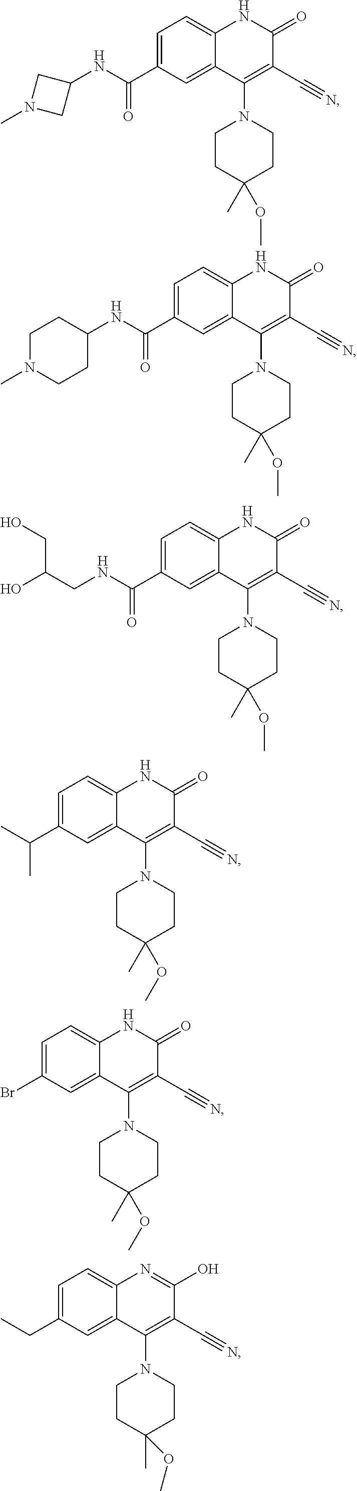

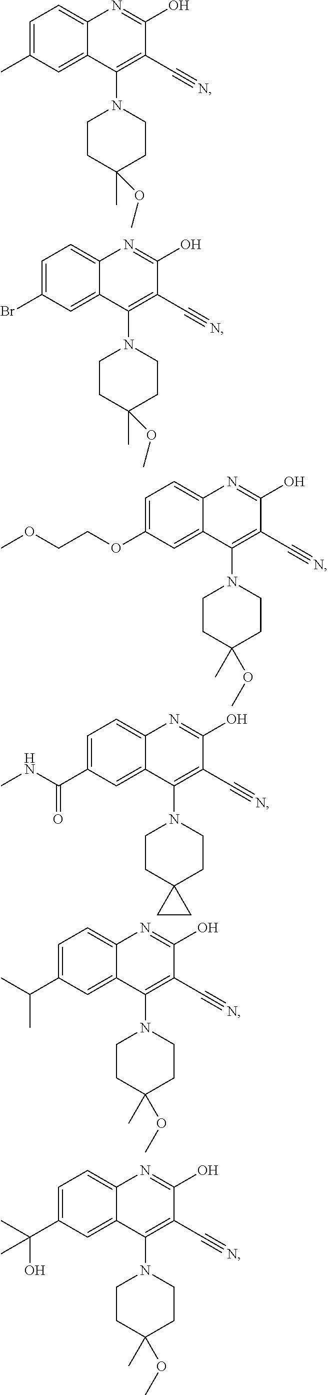

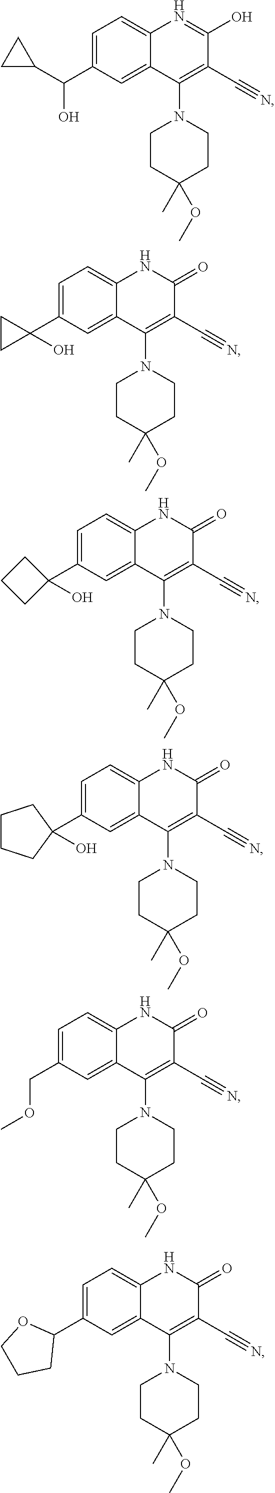

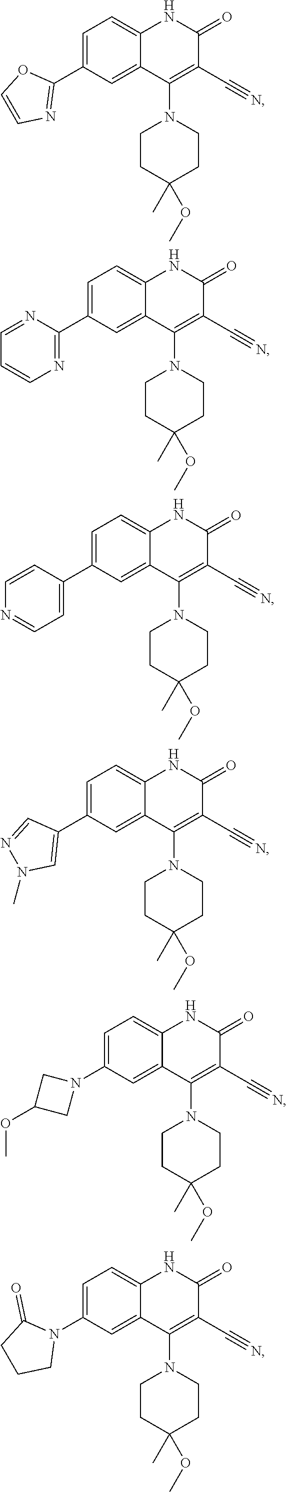

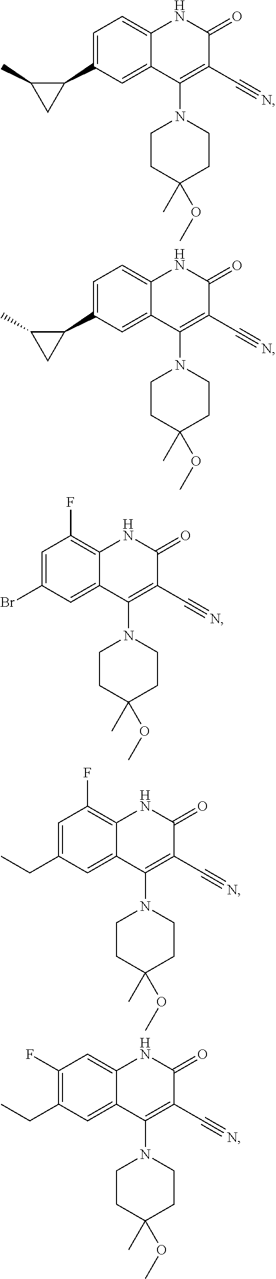

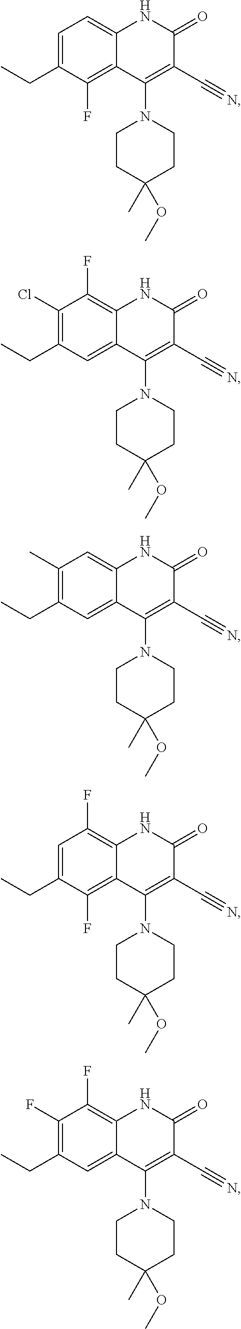

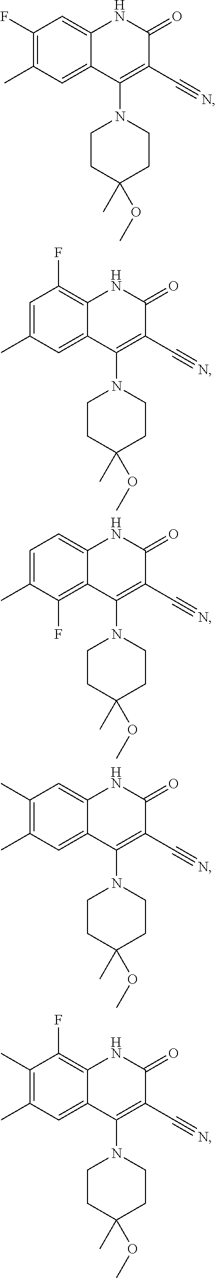

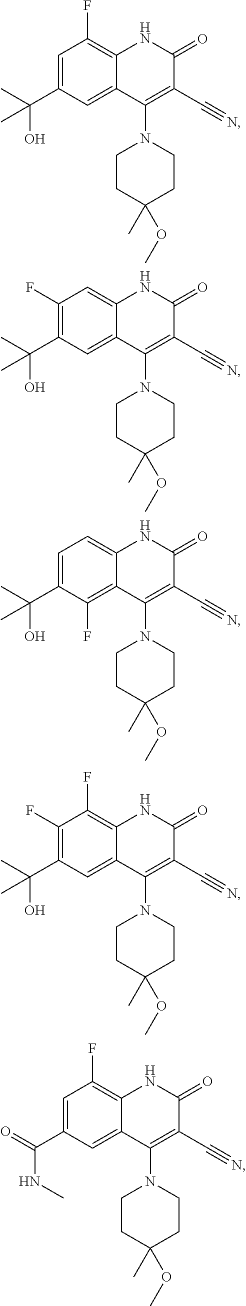

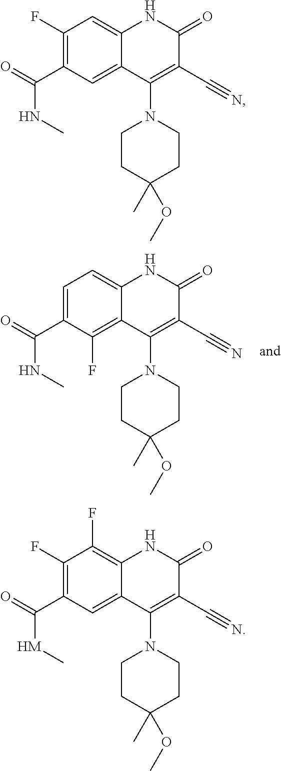

14. A pharmaceutical composition comprising the compound or the pharmaceutically acceptable salt or the isomer thereof according to any one of claims 1-13, and one or more second therapeutically active agents, wherein the second therapeutically active agent is selected from acetylcholinesterase inhibitors, amyloid-β (or fragments thereof), antibodies of amyloid-β (or fragments thereof), amyloid-lowering or -inhibiting agents, α-adrenoceptor antagonists, β-adrenoceptor blockers, anticholinergics, anticonvulsants, tranquilizers, calcium channel blockers, catechol-O-methyltransferase inhibitors, central nervous system stimulators, corticosteroids, dopamine receptor agonists, dopamine receptor antagonists, dopamine reuptake inhibitors, γ-aminobutyric acid receptor agonists, immunomodulators, immunosuppressants, interferons, levodopa, N-methyl-D-aspartate receptor antagonists, monoamine oxidase inhibitors, muscarinic receptor agonists, nicotinic receptor agonists, neuroprotective agents, norepinephrine reuptake inhibitors, other PDE9 inhibitors, other phosphodiesterase inhibitors, β-secretase inhibitors, γ-secretase inhibitors, serotonin (5-hydroxytryptamine)1A (5-HT_{1A}) receptor antagonists, serotonin (5-hydroxytryptamine)6 (5-HT₆) receptor antagonists, serotonin (5-HT) reuptake inhibitors and trophic factors.

15. A pharmaceutical formulation comprising the compound or the pharmaceutically acceptable salt or the isomer thereof according to any one of claims 1-13, wherein the pharmaceutical formulation comprises one or more pharmaceutical carriers.

16. Use of the compound or the pharmaceutically acceptable salt or the isomer thereof according to any one of claims 1-13, the pharmaceutical composition according to claim 14 or the pharmaceutical formulation according to claim 15 in the manufacture of a medicament for treating or preventing the PDE9-mediated related disease.
